(19) United States
(12) Patent Application Publication
(10) Pub. No.: US 2004/0254132 A1
(43) Pub. Date: Dec. 16, 2004

(54) Ovary-specific genes and proteins

(76) Inventors: Martin M. Matzuk, Pearland, TX (US); Xuemei Wu, Houston, TX (US); Pei Wang, Houston, TX (US); Yuchen Bai, Newton, PA (US)

Correspondence Address: FULBRIGHT & JAWORSKI, LLP, 1301 MCKINNEY, SUITE 5100, HOUSTON, TX, 77010-3095, US

(21) Appl. No.: 10/475,502

(22) PCT Filed: Apr. 26, 2002
(86) PCT No.: PCT/US02/13245

(30) Foreign Application Priority Data
Apr. 27, 2001 (US) 09844864

(51) Int. Cl. A61K 48/00; C07H 21/04

(52) U.S. Cl. 514/44; 536/23.2

(57) ABSTRACT

The present invention relates generally to ovary-specific genes (O1-180, O1-184 and O1-236) and the proteins they encode. Also provided are methods for detecting cell proliferative or degenerative disorders in reproductive tissues. Yet further, the invention provides methods for screeing of compounds that interact and/or modulate the expression or activity of the ovary-specific genes. These compounds are possible contraceptive agents and/or fertility agents.

This application is a continuation-in-part of U.S. application Ser. No. 09/844,864, which was filed on Apr. 27, 2001, which is a continuation-in-part application of International Application Number PCT/US99/25209 filed Oct. 28, 1999, which is an international application claiming priority to U.S. Provisional Application No. 60/106,020 filed Oct. 28, 1998.

### BACKGROUND OF THE INVENTION

A. Field of the Invention

The present invention relates generally to ovary-specific genes and the proteins they encode.

B. Description of Related Art

Reproductive development and function are complex processes involving both genetically-determined and physiological events. Identification of the critical protein products of genes involved in these processes is necessary to characterize how these processes are regulated. Although important molecular events occur during the early phases of mammalian oogenesis and folliculogenesis, to date, few “candidate” regulatory molecules have been identified and characterized thoroughly. Several studies have suggested that both endocrine factors, such luteinizing hormone (LH) and follicle stimulating hormone (FSH) from the pituitary, as well as paracrine factors secreted from the oocyte influence folliculogenesis. FSH and LH are known to bind to granulosa and thecal cells which in turn are required for oocyte growth and maturation and maintenance of oocyte meiotic competence. Likewise, oocytes may secrete factors which are necessary for normal granulosa cell and thecal cell function. Because oocyte growth is coordinated with the development and growth of the surrounding somatic cells (i.e., granulosa cells initially and thecal cells later), understanding the molecular events at early stages will give important clues about the paracrine factors mediating the reciprocal interactions between oocytes and somatic cells, the development of competence for trophic hormone stimulation, and the process of follicular recruitment.

Disruption of the hypothalamic-pituitary-gonadal reproductive axis by administration of steroids containing synthetic estrogens and progestins has been one of the oldest methods of hormonal contraception. However, the latest report of the Institute of Medicine emphasizes the importance of developing strategies for new contraceptives. According to the report, some of the long-term contraceptive strategies for women include inhibition of ovulation, prevention of fertilization, or blocking of implantation of a fertilized egg into the uterine lining. Furthermore, infertility affects ˜15% of couples, and in 40% of the cases, the female is believed to be the sole cause of the infertility. Thus, it is critical to identify novel ovary-specific gene products which could be potential targets for new contraceptive agents.

One function of the ovary is to produce an oocyte that is fully capable of supplying all the necessary proteins and factors for fertilization and early embryonic development. Oocyte-derived mRNA and proteins are necessary for the removal of the sperm nuclear envelope, the decondensation of the sperm nucleus (including the removal of protamines), the assembly of histones on the sperm DNA and chromatin condensation, the completion of oocyte meiotic maturation and extrusion of the second polar body, the formation of male and female pronuclei, the fusion of male and female pronuclei, the replication of DNA, and the initiation of zygote and early embryonic cleavages [reviewed in (Perreault, 1992)]. Oocyte-derived factors are necessary since the sperm contains mainly DNA (i.e., no cytoplasm or nucleoplasm), and many of the factors necessary for early post-fertilization events in mammals are acquired during oocyte meiotic maturation (McLay and Clarke, 1997). These oocyte proteins are predicted to be highly conserved through evolution since oocytes can efficiently remodel heterologous sperm or somatic cell nuclei into pronuclei (Perreault, 1992). Although histones are involved in the modification of the sperm chromatin to resemble that of a somatic cell, the other non-histone proteins involved in these processes are unknown in mammals. In Xenopus laevis, a key factor in sperm decondensation is nucleoplasmin which was isolated and cloned over a decade ago (Burglin et al., 1987; Dingwall et al., 1987). Sperm chromatin decondensation occurs after a spermatotozoon enters an egg. In Xenopus laevis, although reduction of the protamine disulfide bonds by ooplasmic glutathione is important, nucleoplasmin (also called nucleoplasmin A or Xnpm2) is necessary and sufficient to initiate the decondensation of sperm nuclei (Philpott et al., 1991). Nucleoplasmin, an acidic, thermostable protein, is the most abundant protein in the nucleus of Xenopus laevis oocytes and eggs, making up 7-10% of the total nuclear protein (Krohne and Franke, 1980a; Mills et al., 1980). After germinal vesicle breakdown, nucleoplasmin [present in the egg nucleoplasm but not bound to DNA Mills et al., 1980)], is released into the ooplasm where it functions to bind protamines tightly and strip them from the sperm nucleus within 5 minutes of sperm entry, resulting in sperm decondensation (Ohsumi and Katagiri, 1991; Philpott and Leno, 1992; Philpott et al., 1991). This process allows egg histones to subsequently bind the sperm DNA. Immunodepletion of nucleoplasmin from egg extracts prevents sperm decondensation (Philpott et al., 1991). Direct interaction of nucleoplasmin with protamine was observed in in vitro experiments, which suggest that the nucleoplasmin is bound to protamine in a 1:1 ratio and that the polyglutamic acid tract in nucleoplasmin plays a critical role for binding to protamine (Iwata et al., 1999). Interestingly, injection of sperm DNA into oocyte nuclei, male or female pronuclei of fertilized eggs, or nuclei of 2 cell embryos leads to sperm decondensation (Maeda et al., 1998), suggesting that nucleoplasmin is functional at all of these stages. Nucleoplasmin can also interact with histones as a pentamer (Earnshaw et al., 1980; Laskey et al., 1993). Nucleoplasmin binds specifically to histones H2A and H2B and along with the proteins N1/N2 that bind histones H3 and H4, can promote nucleosome assembly onto DNA (Dilworth et al., 1987; Laskey et al., 1993). Thus, these observations suggest that during oogenesis and during oogenesis and at fertilization, the oocyte-derived nucleoplasmin interacts with the female pronucleus and male pronucleus, interacts with histones, and is required in some way for chromatin assembly. (Laskey et al., 1993; Philpott et al., 1991). Although “ubiquitous” proteins with low homology to nucleoplasmin have been cloned in mammals and Drosophila (Chan et al., 1989; Crevel et al., 1997; Ito et al., 1996; MacArthur and Shackleford, 1997b; Schmidt-Zachmann and Franke, 1988), an oocyte-equivalent ortholog in mammals had not yet been identified.

The basic functional unit within the ovary is the follicle, which consists of the oocyte and its surrounding somatic cells. Fertility in female mammals depends on the ability of the ovaries to produce Graafian (pre-ovulatory) (pre-ovulatory) follicles, which ovulate fertilizable oocytes at mid-cycle (Erickson and Shimasaki, 2000). This process, termed folliculogenesis, requires a precise coordinate regulation between extraovarian and intraovarian factors (Richards et al., 1995). Compared to the knowledge of extraovarian regulatory hormones at the levels of the hypothalamus (i.e., GnRH) and anterior pituitary (i.e., FSH and LH), little is known about paracrine and autocrine factors within the ovaries, though oocyte-somatic cell communication has been long recognized as important (Falck, 1959). Accumulating evidence shows that factors secreted by the oocyte promote the proliferation of surrounding granulosa cells, and inhibit premature luteinization of these cells during folliculogenesis (El-Fouly et al., 1970; Channing, 1970). Oocyte factors have been implicated in controlling granulosa cell synthesis of hyaluronic acid, urokinase plasminogen activator (uPA), LH receptor, steroidsand prostaglandins and prostaglandins (El-Fouly et al., 1970; Nekola and Nalbandov, 1971; Salustri et al., 1985; Vanderhyden et al., 1993; Eppig et al., 1997a, b).

Several novel regulatory proteins have been recently discovered within oocytes. Growth differentiation factor 9 (GDF-9 or Gdf9), a member of transforming growth factor β (TGF-β) superfamily, is one of the most important signaling factors. Oocyte expression of GDF-9 begins at the primary follicle stage, and persists through ovulation in the mouse (McGrath et al., 1995; Elvin et al., 2000). Female Gdf9 knockout mice are infertile due to a block of folliculogenesis at the type 3b (primary) follicle stage, accompanied by defects in granulosa cell growth and differentiation, theca cell formation, and oocyte meiotic competence (Dong et al., 1996; Carabatsos et al., 1998, Elvin et al., 1999A). Also, recombinant GDF-9 affects the expression of the genes encoding hyaluronan synthase 2 (Has2), cyclooxygenase 2 (Cox2), steroid acute regulatory protein (StAR), the prostaglandin E2 receptor EP2, pentaraxin 3, LH receptor and uPA (Elvin et al., 1999B, Elvin et al., 2000).

To identify key proteins in the hypothalamic-pituitary-gonadal axis, several important knockout mouse models have been generated, including four which have ovarian defects. Mice lacking gonadal/pituitary peptide inhibin have secondary infertility due to the onset of ovarian or testicular tumors which appear as early as 4 weeks of age (Matzuk, et al., 1992). Mice lacking activin receptor type II (Acvr2) survive to adulthood but display reproductive defects. Male mice show reduced testes size and demonstrate delayed fertility (Matzuk, et al. 1995). In contrast, female mice have a block in folliculogenesis at the early antral follicle stage leading to infertility. Consistent with the known role of activins in FSH homeostasis, both pituitary and serum FSH levels are dramatically reduced in these Acvr2 knockout mice. Female mice lacking FSH, due to a mutation in the FSHbeta gene, are infertile (Kumar et al., 1997). However, these mice have an earlier block in folliculogenesis prior to antral follicle formation. Thus, FSH is not required for formation of a multi-layer pre-antral follicle, but it is required for progression to antral follicle formation. Finally, growth differentiation factor 9(Gdf9) knockout mice have been used to determine at which stage in follicular development GDF-9 is required (Dong et al., 1996). Within the ovary, expression of Gdf9 mRNA is limited to the oocyte and is seen at the early one-layer primary follicle stage and persists through ovulation. Absence of GDF-9 results in ovaries that fail to demonstrate any normal follicles beyond the primary follicle stage. Although oocytes surrounded by a single layer of granulosa cells are present and appear normal histologically, no normal two-layered follicles are present. Follicles beyond the one-layer stage are abnormal, contain atypical granulosa cells, and display asymmetric growth of these cells. Furthermore, as determined by light and electron microscopy, a thecal cell layer does not form in these Gdf9 knockout ovaries (Dong et al., 1996; Elvin et al., 1999). Thus, in contrast to kit ligand and other growth factors which are synthesized by the somatic cells and influence oocyte growth, GDF-9 function's in the reciprocal manner as an oocyte-derived growth factor which is required for somatic cell function.

### BRIEF SUMMARY OF THE INVENTION

The present invention provides three ovary-specific and oocyte-specific polynucleotide sequences, O1-180 (SEQ.ID.NO.1, SEQ.ID.NO.11, SEQ.ID.NO.12, SEQ.ID.NO.13), O1-184 (SEQ.ID.NO.3) and O1-236 (SEQ.ID.NO.5, SEQ.ID.NO.7, SEQ.ID.NO.8; SEQ.ID.NO.10, and SEQ.ID.NO.14), the protein products they encode, fragments and derivatives thereof, and antibodies which are immunoreactive with these protein products. These genes and their protein products appear to relate to various cell proliferative or degenerative disorders, especially those involving ovarian tumors, such as germ cell tumors and granulosa cell tumors, or infertility, such as premature ovarian failure.

Thus, in one embodiment, the invention provides methods for detecting cell proliferative or degenerative disorders of ovarian origin and which are associated with O1-180, O1-184 or O1-236. In another embodiment, the invention provides method of treating cell proliferative or degenerative disorders associated with abnormal levels of expression of O1-180, O1-184 or O1-236, by suppressing or enhancing their respective activities.

In a specific embodiments, the present invention provides a pharmaceutical composition comprising a modulator of O1-180, O1-184 and/or O1-236 expression dispersed in a pharmaceutically acceptable carrier. The modulator may suppress or enhance transcription of an O1-180, O1-184 and/or O1-236 gene. The modulator may be a polypeptide sequence, a protein, a small molecule, or a polynucleotide sequence. Specifically, the polynucleotide sequence is DNA or RNA. In further embodiments, the polynucleotide sequence is comprised in an expression vector operatively linked to a promoter.

A further embodiment of the present invention is a pharmaceutical composition comprising a modulator of O1-180, O1-184 and/or O1-236 activity dispersed in a pharmaceutically acceptable carrier. The composition may inhibit or stimulate O1-180, O1-184 and/or O1-236 activity. The composition may be a protein, polypeptide sequence, small molecule, or polynucleotide sequence.

Another embodiment of the present invention is a method of modulating contraception comprising administering to an animal an effective amount of a modulator of O1-180, O1-184 and/or O1-236 activity dispersed in a pharmacologically acceptable carrier, wherein said amount is capable of decreasing conception. The animal may be a male or a female.

A further embodiment is a method of enhancing fertility comprising administering to an animal an effective amount of a modulator of O1-180, O1-184 and/or O1-236 activity dispersed in a pharmacologically acceptable carrier, wherein said amount is capable of increasing conception.

Yet further, another embodiment is a method of screening for a modulator of O1-180, O1-184 and/or O1-236 activity comprising the steps of: providing a cell expressing an O1-180, O1-184 and/or O1-236 polypeptide; contacting said cell with a candidate modulator; measuring O1-180, O1-184 and/or O1-236 expression; and comparing the O1-180, O1-184 and/or O1-236 expression in the presence of the candidate modulator with the expression of O1-180, O1-184 and/or O1-236 expression in the absence of the candidate modulator; wherein a difference in the expression of O1-180, O1-184 and/or O1-236 in the presence of the candidate modulator, as compared with the expression of O1-180, O1-184 and/or O1-236 in the absence of the candidate modulator, identifies the candidate modulator as a modulator of O1-180, O1-184 and/or O1-236 expression.

A specific embodiment of the present invention is a method of identifying compounds that modulate the activity of O1-180, O1-184 and/or O1-236 comprising the steps of obtaining an isolated O1-180, O1-184 and/or O1-236 polypeptide or functional equivalent thereof; admixing the O1-180, O1-184 and/or O1-236 polypeptide or functional equivalent thereof with a candidate compound; and measuring an effect of said candidate compound on the activity of O1-180, O1-184 and/or O1-236.

Another embodiment is method of screening for a compound which modulates the activity of O1-180, O1-184 and/or O1-236 comprising exposing O1-180, O1-184 and/or O1-236 or a O1-180, O1-184 and/or O1-236 binding fragment thereof to a candidate compound; and determining whether said compound binds to O1-180, O1-184 and/or O1-236 or the O1-180, O1-184 and/or O1-236 binding partner thereof; and further determining whether said compound modulates O1-180 or the O1-180 interaction with a binding partner.

Yet further, another embodiment is a method of screening for an interactive compound which binds with O1-180, O1-184 and/or O1-236 comprising exposing a O1-180, O1-184 and/or O1-236 protein, or a fragment thereof to a compound; and determining whether said compound bound to the O1-180, O1-184 and/or O1-236.

Another embodiment is a method of identifying a compound that effects O1-180, O1-184 and/or O1-236 activity comprising providing a group of transgenic animals having (1) a regulatable one or more O1-180, O1-184 and/or O1-236 protein genes, (2) a knock-out of one or more O1-180, O1-184 and/or O1-236 protein genes, or (3) a knock-in of one or more O1-180, O1-184 and/or O1-236 protein genes; providing a second group of control animals respectively for the group of transgenic animals; and exposing the transgenic animal group and control animal group to a potential O1-180, O1-184 and/or O1-236-modulating compounds; and comparing the transgenic animal group and the control animal group and determining the effect of the compound on one or more proteins related to infertility or fertility in the transgenic animals as compared to the control animals.

In specific embodiments, the present invention provides a method of detecting a binding interaction of a first peptide and a second peptide of a peptide binding pair, comprising culturing at least one eukaryotic cell under conditions suitable to detect the selected phenotype; wherein the cell comprises; a nucleotide sequence encoding a first heterologous fusion protein comprising the first peptide or a segment thereof joined to a transcriptional activation protein DNA binding domain; a nucleotide sequence encoding a second heterologous fusion protein comprising the second peptide or a segment thereof joined to a transcriptional activation protein transcriptional activation domain; wherein binding of the first peptide or segment thereof and the second peptide or segment thereof reconstitutes a transcriptional activation protein; and a reporter element activated under positive transcriptional control of the reconstituted transcriptional activation protein, wherein expression of the reporter element produces a selected phenotype; detecting the binding interaction of the peptide binding pair by determining the level of the expression of the reporter element which produces the selected phenotype; wherein said first or second peptide is an O1-180, O1-184 and/or O1-236 peptide and the other peptide is a test peptide, preferably selected peptides/proteins present in a reproductive tissue. In specific embodiments the reproductive tissue is an ovary or testis. Other reproductive tissues may also include the uterus, vagina, oviduct, cerivx, gonads, vas deferens, prostate, seminal vesicles and epididymis.

A further embodiment is a rescue screen for detecting the binding interaction of a first peptide and a second peptide of a peptide binding pair, comprising: culturing at least one eukaryotic cell under conditions to detect a selected phenotype or the absence of such phenotype, wherein the cell comprises; a nucleotide sequence encoding a first heterologous fusion protein comprising the first peptide or a segment thereof joined to a DNA binding domain of a transcriptional activation protein; a nucleotide sequence encoding a second heterologous fusion protein comprising the second peptide or a segment thereof joined to a transcriptional activation domain of a transcriptional activation protein; wherein binding of the first peptide or segment thereof and the second peptide or segment thereof reconstitutes a transcriptional activation protein; and a reporter element activated under positive transcriptional control of the reconstituted transcriptional activation protein, wherein expression of the reporter element prevents exhibition of a selected phenotype; detecting the ability of the test peptide to interact with O1-180, O1-184 and/or O1-236 by determining whether the test peptide affects the expression of the reporter element which prevents exhibition of the selected phenotype, wherein said first or second peptide is an O1-180, O1-184 and/or O1-236 peptide and the other peptide is a test peptide, preferably selected peptides/proteins present in a reproductive tissue. In specific embodiments, the reproductive tissue is an ovary or testis.

Yet further, another embodiment is a method of identifying binding partners for O1-180, O1-184 and/or O1-236 comprising the steps of: exposing the protein to a potential binding partner; and determining if the potential binding partner binds to O1-180, O1-184 and/or O1-236.

The present invention provides key in vitro and in vivo reagents for studying ovarian development and function. The possible applications of these reagents are far-reaching, and are expected to range from use as tools in the study of development to therapeutic reagents against cancer. The major application of these novel ovarian gene products is to us them as reagents to evaluate potential contraceptives to block ovulation in women in a reversible or irreversible manner. It will also be expected that these novel ovarian gene products will be useful to screen for genetic mutations in components of these signaling pathways that are associated with some forms of human infertility or gynecological cancers or other cancers associated with reproductive tissues. In addition, depending on the phenotypes of humans with mutations in these genes or signaling pathways, the inventors may consider using these novel ovarian gene products as reagent tools to generate a number of mutant mice for the further study of oogenesis, folliculogenesis, and/or early embryogenesis as maternal effect genes. Such knockout mouse models will provide key insights into the roles of these gene products in human female reproduction and permit the use of these gene products as practical reagents for evaluation of new contraceptives.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIG. 1. Multi-tissue Northern blot analysis of ovary-specific genes. Northern blot analysis was performed on total RNA using O1-180, O1-184, and O1-236 probes. These gene products demonstrate an ovary-specific pattern (OV, ovary; WT, wild-type; −/−, Gdf9 knockout) as shown. The migration positions of 18S and 28S ribosomal RNA are indicated. All lanes had approximately equal loading as demonstrated using an 18S rRNA cDNA probe. Br, brain; Lu, lung; He, heart; St, stomach; Sp, spleen; Li, liver; Si, small intestine; Ki, kidney; Te, testes, Ut, uterus.

FIGS. 2A-2F. In situ hybridization analysis of ovary-specific genes in mouse ovaries. In situ hybridization was performed using anti-sense probes to O1-180 (FIGS. 2A-2B), O1-184 (FIGS. 2C-2D) and O1-236 (FIGS. 2E-2F). FIGS. 2A, 2C, and 2E are brightfield analysis of the ovaries. FIGS. 2B, 2D, and 2F are darkfield analysis of the same ovary sections. All genes demonstrate specific expression in the oocyte beginning at the one layer primary follicle stage (small arrows) and continuing through the antral follicle stage (large arows).

FIGS. 3A and 3B. In situ hybridization analysis of O1-236 in mouse ovaries. In situ hybridization was performed using probe O1-236 (partial Npm2 cDNA fragment). Brightfield analysis (FIG. 3A) and darkfield analysis (FIG. 3B) of the O1-236 mRNA in the same adult ovary sections. The probe demonstrates specific expression in all growing oocytes. Oocyte-specific expression is first seen in the early one layer primary follicle (type 3a), with higher expression in the one layer type 3b follicle and all subsequent stages including antral (an) follicles.

FIG. 4. Npm2 cDNA representation. Schematic representation of the mouse Npm2 cDNA sequence (984 bp) and two of the clones isolated from the mouse ovary cDNA libraries. The original O1-236 probe (749 bp) is shown at the top and encompasses the entire Npm2 open reading frame. The open reading frame (solid box) is 621 bp and the 5′ UTR and 3′ UTR sequences (thin lines) are 155 bp and 205 bp, respectively. The polyA sequences are not depicted. Clone 236-1 (Npm2) was isolated from the wild-type ovary cDNA library and clone 236-3 was isolated from the Gdf9 knockout ovary cDNA library. Clone 236-3 (984 bp excluding polyA sequence) is 4 bp longer at the 5′ end and 1 bp longer at the 3′ end than clone 236-1 (979 bp excluding polyA sequences).

FIG. 5. Amino acid sequence conservation among Xenopus laevis (SEQ.ID.NO.15), mouse (SEQ.ID. NO.6), and human (SEQ.ID.NO.9) NPM2 proteins. Using the NCBI blast search tools and Megalign software, comparison of mouse (m), human (h), and Xenopus laevis NPM2 amino acid sequences reveals high identity (amino acids highlighted in blue). Spaces between the amino acids indicate gaps to aid in the alignment. Also identified are the conserved bipartite nuclear localization signal (red), the highly acidic histone and protamine binding region (red), and several conserved casein kinase II (CK2) and protein kinase C (PKC) phosphorylation sites (underlined and marked with “CK” or “PKC”). Other predicted phosphorylation sites in the nucleoplasmins, which are not conserved, are not shown.

FIG. 6A and FIG. 6B. Structure of the mouse Npm2 gene (FIG. 6A). Two overlapping recombinant lambda clones (236-13 and 236-14), isolated from a mouse 129/SvEv library, are shown at the top, and a schematic enlargement of the Npm2 gene is also depicted. Open boxes represent untranslated regions and solid black boxes represent protein coding regions. The 236-13 insert is ˜19.0 kb and 236-14 insert is ˜21.0 kb. The entire contig is ˜37 kb. All 9 exons of the Npm2 gene are encompassed on a single 6.9 kb Xbal (X) fragment as shown. The size of exons and introns are shown at the bottom. Abbreviations: B, BamH1; (B), predicted but unmapped BamH1; (N), NotI from phage cloning site. FIG. 6B shows the structure of the human Npm2 gene.

FIGS. 7A and 7B. Mouse Npm2 gene (SEQ ID NO: 7) and amino acid sequences (SEQ.ID.NO.6). Uppercase letters represent sequence identity with the Npm2 cDNA sequences; non-transcribed 5′ and 3′ sequences and intron sequences are shown in lowercase. The predicted transcription initiation codon, the termination codon, and the polyadenylation signal sequence are all underlined. Numbers along the left side represent the amino acids. The underlined and bolded “T” in codon 36, the bolded “c” for amino acid 26, and the underlined and bolded “C: in the 3′ UTR sequence indicate differences between the cDNA and gene sequences. Arrows indicate where the O1-236 fragment initiates and ends in the cDNA sequence.

FIG. 8. Chromosomal localization of the mouse Npm2 gene. (Top) Map figure from the T31 radiation hybrid database at The Jackson Laboratory showing Chromosome 14 data. The map is depicted with the centromere toward the top. Distances between adjacent loci in centiRay3000 are shown to the left of the chromosome bar. The positions of some of the chromosome 14 MIT markers are shown on the right. The mouse Npm2 gene is positioned between D14Mit203 and D14Mit32. Missing typings were inferred from surrounding data where assignment was unambiguous. (Bottom) Haplotype figure from the T31 radiation hybrid database at The Jackson Laboratory showing part of Chromosome 14 with loci linked to Npm2. Loci are listed in the best fit order with the most proximal at the top. The black boxes represent hybrid cell lines scoring positive for the mouse fragment and the white boxes represent cell lines scoring as negative. The grey box indicates an untyped or ambiguous line. The number of lines with each haplotype is given at the bottom of each column of boxes. Missing typings were inferred from surrounding data where assignment was unambiguous.

FIGS. 9A-9H. Analysis of Npm2 mRNA and NPM2 protein in mouse ovaries and early embryos. In situ hybridization was performed using probe O1-236 (partial Npm2 cDNA fragment). Brightfield analysis (FIG. 9A) and darkfield analysis (FIG. 9B) of the O1-236 mRNA in the same adult ovary sections. (FIG. 9C) Imunohistochemistry of ovaries from a 5-week old mouse stained for NPM2 in the nuclei (bright red) of oocytes from type 3 (arrow) to antral follicles. (FIG. 9D) In preovulatory GVB oocytes induced by luteinizing hormone (hCG), NPM2 is evenly stained in the cytoplasm (arrow). An LH (hCG) unresponsive preantral follicle (upper right) continues to demonstrate an oocyte with NPM2 protein localized to the nucleus. (FIG. 9E) After fertilization, NPM2 begins to localize in the pronuclei; the formation of one pronucleus (arrow), is in the process of forming and some of NPM2 staining continues to be present in the cytoplasm of this early one cell embryo. (FIG. 9F) The pronuclei stain strongly in an advanced one cell embryo where very little NPM2 remains in the cytoplasm. NPM2 antibodies also specifically stain the nuclei of two cell (FIG. 9G) and eight cell (FIG. 9H) embryos.

FIGS. 10A-10C. Gene targeting construct for a knockout of Npm2 and genotype analysis of offspring from heterozygote intercrosses. (FIG. 10A) The targeting strategy used to delete exon 2, exon 3, and the junction region of exon 4. PGK-hprt and MC1-tk expression cassettes are shown. Recombination were detected by Southern blot analysis using 5′ and 3′ probes. (B, BamH1; Bg, Bgl II; P, Pst I). (FIG. 10B) Southern blot analysis of genomic DNA isolated from mice generated from intercrosses of Npm2^{+/-} mice. The 3′ probe identifies the wild-type 7.5-kb band and the mutant 10.3-kb band when DNA was digested with Bgl II. (FIG. 10C) When DNA was digested with Pst 1, the exon 2 probe against only detected the wild-type 4.5-kb fragment.

FIGS. 11A-11F. Histological analysis of ovaries from wild-type, Npm2^{+/-}, and Npm2^{-/-} mice. (FIG. 11A-11D) Immunohistochemistry of ovaries from 6-week old mice stained for Npm2 in the nuclei of oocytes (FIG. 11A and FIG. 11C for Npm2^{+/-} ovaries; FIG. 11B and FIG. 11D for Npm2^{-/-} ovaries). (FIG. 11E-11F) PAS (Periodic acid Schiff)/hematoxylin staining of ovaries from 12 week old mice wild-type (FIG. 11E) and Npm2^{-/-} (FIG. 11F) ovaries. Arrows show large antral follicles; “CL” denote corpora lutea.

FIGS. 12A-12D. In vitro culture of eggs and fluorescent-labeling of DNA from fertilized eggs from Npm2^{-/-} and control mice. Eggs were isolated from the oviducts of immature mice after superovulation and cultured in vitro. Pictures were taken under a microscope at 24 and 48 hours of culture. (FIGS. 12A, 12C) Most of the eggs from wild-type mice divided to form two cell embryos by 24 h; some of two cell embryos progressed to the four cell stage after 48 h of culture. (FIGS. 12B, 12D) Very few eggs from Npm2^{-/-} mice cleaved into two cell embryos; no four cell embryos were detected after 48 hours of culture. Some developmentally abnormal or apparently apoptosed embryos from Npm2^{-/-} mice were detected.

FIGS. 13A-13F. Localization of O1-180 in mouse ovaries. Expression of O1-180 in PMSG-treated wild-type (FIGS. 13A and 13B) and Gdf9 knockout (FIGS. 13C-13F) ovaries was analyzed by in situ hybridization with a specific antisense probe. The expression of O1-180 gene was detectable at early primary follicle stage (type 3a) through ovulatory follicle stage, but not in primordial follicles in wild-type ovaries. In Gdf9 knockout ovaries, the follicle numbers was increased per unit volume due to the arrest of follicle development at primary follicle stage, more O1-180 positive signal were detected in each section. FIGS. 13A, 13C and 13E, brightfield analysis of the ovaries; FIGS. 13B, 13D and 13F, corresponding darkfield analysis of the same ovary sections. FIGS. 13E and 13F were high power magnification of the same sections shown in FIGS. 13C and 13D.

FIG. 14. Structure of the O1-180 (SEQ.ID.NO.11) gene and O1-180 (SEQ.ID.NO.12) pseudogene. Diagrams representing the O1-180 pseudogene and the O1-180 gene are shown at the top along with unique restriction endonucleases sites which were important in constructing the linear map shown at the bottom. Exons and introns are drawn to scale. Boxes denote exons, hatched regions denote protein coding portions and the solid regions denote the untranslated portions. Lines connecting boxes denote introns. O1-180ps: O1-180 pseudogene; O1-180: O1-180 gene; B: BamHI; S: SalI; X: XhoI;

FIGS. 15A and 15D. Comparison of O1-180 gene and O1-180 pseudogene. Sequences of exons, exon-intron boundaries and the size of each intron are shown. Different nucleotides between the two genes and consensus polyadenylation sequence are underlined. The translation start codon and stop codon are shown in bold. Upper case: exon sequences; lower case: intron sequences.

FIG. 16. Maps of mouse chromosome 5, showing the position in centiMorgan (cM) of the marker best linked to O1-180 gene (A) and its related pseudogene (B) (data and maps generated at the Jackson Laboratory Bioinformatics Server).

FIG. 17. Gene targeting constructs for O1-180. The targeting strategy used to delete exon 1. PGK-hprt and MC1-tk expression cassettes are shown.

FIG. 18. Northern blot analysis of O1-180 mRNA expression in multiple tissues.

FIG. 19. Western blot analysis of recombinant O1-180.

FIGS. 20A-20F. Immunostaning of O1-180 in mouse ovaries. Anti-O1-180 polyclonal antibodies (made in goats) were used for IHC to detect the expression of O1-180 in mouse ovary sections. FIG. 20A-FIG. 20D are wild-type ovaries; FIG. 20E-FIG. 20F are Gdf9 knockout ovaries. FIG. 20b is a negative control with normal goat serum. The O1-180 protein was localized specifically to the cytoplasm of mouse oocytes and zygotes but disappears after this point. Staining indicates the location of the O1-180 protein.

FIGS. 21A AND 21B. In vitro culture of O1-190 mouse embryos. FIG. 21A shows embryos cultured from O1-180^{+/-} mice and FIG. 21B shows embryos cultured from O1-180^{-/-} mice. On the third day of in vitro culture in M16 medium, most control embryos progressed to the morula or blastocyst stage, while zygotes in O1-180 knockout mice still remained at the one-cell or two-cell stage.

FIG. 22. This figure shows a comparison of the human and mouse O1-180 proteins. The differences are underlined. The proteins have a similarity of 91.3%.

### DETAILED DESCRIPTION OF THE INVENTION

It is readily apparent to one skilled in the art that various embodiments and modifications can be made to the invention disclosed in this Application without departing from the scope and spirit of the invention.

As used herein, the use of the word “a” or “an” when used in conjunction with the term “comprising” in the sentences and/or the specification may mean “one,” but it is also consistent with the meaning of “one or more,” “at least one,” and “one or more than one.”

As used herein, the term “animal” refers to a mammal, such as human, non-human primates, horse, cow, elephant, cat, dog, rat or mouse. In specific embodiments, the animal is a human.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting. Thus, one of skill in the art understands that the term “antibody” refers to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. (See, e.g., Antibodies: A Laboratory Manual, Cold Spring Harbor Laboratory, 1988).

As used herein, the term “binding protein” refers to proteins that demonstrate binding affinity for a specific ligand. Binding proteins may be produced from separate and distinct genes. For a given ligand, the binding proteins that are produced from specific genes are distinct from the ligand binding domain of the receptor.sub.r or its soluble receptor

As used herein, the term “conception” refers to the union of the male sperm and the ovum of the female; fertilization.

As used herein, the term “contraception” refers to the prevention or blocking of conception. A contraceptive device, thus, refers to any process, device, or method that prevents conception. Well known categories of contraceptives include, steroids, chemical barrier, physical barrier; combinations of chemical and physical barriers; use of immunocontraceptive methods by giving either antibodies to the reproductive antigen of interest or by developing a natural immune response to the administered reproductive antigen; abstinence and permanent surgical procedures. Contraceptives can be administered to either males or females.

As used herein, the term “DNA” is defined as deoxyribonucleic acid.

As used herein, the term “DNA segment” refers to a DNA molecule that has been isolated free of total genomic DNA of a particular species. Included within the term “DNA segment” are DNA segments and smaller fragments of such segments, and also recombinant vectors, including, for example, plasmids, cosmids, phage, viruses, and the like.

As used herein, the term “expression construct” or “transgene” is defined as any type of genetic construct containing a nucleic acid coding for gene products in which part or all of the nucleic acid encoding sequence is capable of being transcribed can be inserted into the vector. The transcript is translated into a protein, but it need not be. In certain embodiments, expression includes both transcription of a gene and translation of mRNA into a gene product. In other embodiments, expression only includes transcription of the nucleic acid encoding genes of interest. In the present invention, the term “therapeutic construct” may also be used to refer to the expression construct or transgene. One skilled in the art realizes that the present invention utilizes the expression construct or transgene as a therapy to treat infertility. Yet further, the present invention utilizes the expression construct or transgene as a “prophylactic construct” for contraception. Thus, the “prophylactic construct” is a contraceptive.

As used herein, the term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well and are described infra.

As used herein, the term “gene” is used for simplicity to refer to a functional protein, polypeptide or peptide encoding unit. This functional term includes both genomic sequences, cDNA sequences and engineered segments that express, or may be adapted to express, proteins, polypeptides, domains, peptides, fusion proteins and mutant. Thus, one of skill in the art is aware that the term “native gene” refers to a gene as found in nature with its own regulatory sequences and the term “chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences that are derived from the same source, but arranged in a manner different than that found in nature.

As used herein, the term “fertility” refers to the quality of being productive or able to conceive. Fertility relates to both male and female animals.

The term “hyperproliferative disease” is defined as a disease that results from a hyperproliferation of cells. Hyperproliferative disease is further defined as cancer. The hyperproliferation of cells results in unregulated growth, lack of differentiation, local tissue invasion, and metastasis. Exemplary hyperproliferative diseases include, but are not limited to cancer or autoimnune diseases. Other hyperproliferative diseases can include vascular occlusion, restenosis, atherosclerosis, or inflammatory bowel disease.

As used herein, the term “infertility” refers to the inability or diminished ability to conceive or produce offspring. Infertility can be present in either male or female. In the present invention, administration of a composition to enhance infertility or decrease fertility is reversible.

As used herein, the terms “O1-180” and “Oo1” are interchangeable.

As used herein, ther terms “O1-236”, Npm2” or “NPM2” are interchangeable.

As used herein, the term “peptide binding pair” refers to any pair of peptides having a known binding affinity for which the DNA sequence is known or can be deduced. The peptides of the peptide binding pair must exhibit preferential binding for each other over any other components of the modified cell.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the vectors or cells of the present invention, its use in therapeutic and/or prophylactic compositions is contemplated. Supplementary active ingredients also can be incorporated into the compositions.

As used herein, the term “polynucleotide” is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means. Furthermore, one skilled in the art is cognizant that polynucleotides include mutations of the polynucleotides, include but are not limited to, mutation of the nucleotides, or nucleosides by methods well known in the art.

As used herein, the term “polypeptide” is defined as a chain of amino acid residues, usually having a defined sequence. As used herein the term polypeptide is interchangeable with the terms “peptides” and “proteins”.

As used herein, the term “promoter” is defined as a DNA sequence recognized by the synthetic machinery of the cell, or introduced synthetic machinery, required to initiate the specific transcription of a gene.

As used herein, the term “purified protein or peptide”, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

As used herein, the term “RNA” is defined as ribonucleic acid.

As used herein, the term “RNA interference” or “iRNA” is an RNA molecule that is used to inhibit a particular gene of interest.

As used herein, the term “under transcriptional control” or “operatively linked” is defined as the promoter is in the correct location and orientation in relation to the nucleic acid to control RNA polymerase initiation and expression of the gene.

In an effort to identify other novel ovarian-expressed genes that may play key functions in ovarian physiology, fertilization and early cleavage events, the inventors have used a subtractive hybridization approach. Several novel oocyte-expressed genes have been identified by the inventors which are important in regulating oogenesis, folliculogenesis, fertilization, and/or early embryogenesis. One of these oocyte-specific gene products, nucleoplasmin 2 (O1-236 or NPM2), is the mammalian ortholog of Xenopus laevis nucleoplasmin (xNPM2)(Burglin et al., 1987; Dingwall et al., 1987). The 207 amino acid open reading frame of NMP2 demonstrated high homology to the family of proteins called nucleoplasmins or nucleophosmins (nomenclature designation=species). NPM2 human gene, Npm2 mouse gene, and Xnpm2 Xenopus gene; NPM2=protein in all species). Human nucleophosmingene gene (NPMI also called N038; accession # M23613) maps to human chromosome 5q35, encodes a 294 amino acid protein, and has orthologs in mouse (Npm1, also called B23, accession # Q61937) and Xenopus laevis (Xnpm1 or N038 accession # X05496). Mouse nucleoplasmin/nucleophosmin homolog Npm3, which has been mapped to mouse chromosome 19, encodes a protein of 175 amino acids [accession # U64450, (MacArthur and Shackleford, 1997a)], and there is an apparent human NPM3 homolog gene (accession # AF081280). In contrast to Npm2, the genes Npm1 and Npm3 are ubiquitously expressed, and the structure of the mouse Npm2 gene is considerably divergent compared to the mouse Npm3 gene (MacArthur and Shackleford, 1997a).

The Npm2 cDNA sequences have been used by the inventors to obtain the mouse Npm2 gene and the human NPM2 cDNA and gene and also map these genes. Mice lacking Npm2 have defects in fertility due to abnormalities in early post-fertilization cleavage events. The discovery of the mammalian homolog of the most abundant nuclear protein in Xenopus laevis oocytes and eggs (Krohne and Franke, 1980a; Mills et al., 1980) is important for a clear understanding of oogenesis, fertilization, and post-fertilization development in mammals and possibly also to define further oocyte factors which are necessary in mammalian cloning experiments.

Likewise, several studies have shown that phosphorylation of nucleoplasmin influences its function. Comparison of the forms of nucleoplasmin from the oocyte (i.e., in the ovary) versus egg (i.e., after ovulation and ready for fertilization) demonstrate dramatic differences in the level of phosphorylation. Xenopus laevis egg nucleoplasmin is substantially larger than the oocyte form, migrating ˜15,000 daltons larger on SDS-PAGE due to phosphorylation differences (Sealy et al., 1986). Nucleoplasmin has ˜20 phosphate groups/protein in the egg compared to <10 phosphate groups/proteins in the oocyte, and an egg kinase preparation can modify the oocyte nucleoplasmin so it resembles the egg form (Cotten et al., 1986). Functionally, this hyperphosphorylation of nucleoplasmin stimulates its nuclear transport (Vancurova et al., 1995) and also results in a more active form, leading to increased nucleosome assembly (Sealy et al., 1986) and sperm decondensation (Leno et al., 1996). A hyperphosphorylated form of nucleoplasmin is also present during the early stages of Xenopus laevis embryogenesis where it is believed to play some function during the rapid cell cycles and DNA replication (Burglin et al., 1987). The high percentage of serine and threonine residues in frog and mammalian NPM2 suggest a similar role of phosphorylation of mammalian nucleoplasmin 2 in mammalian eggs. Phosphorylation could act to regulate when NPM2 acts, making it inactive until the critical time (i.e., histon addition to maile and femal pronuclei or during transcriptional arrest). Although there are multiple putative kinase sites in both frog and mammalian NPM2, casein kinase II specifically interacts with nucleoplasmin and phosphorylates it, and an inhibitor of casein kinase II can block nuclear transport of Xenopus laevis nucleoplasmin (Vancurova et al., 1995). Interestingly, two of the predicted casein kinase II phosphorylation sites are conserved betweenfrog nucleoplasmin2 (Ser125 and Ser177), mouse NPM2 (Thr123 and Ser184), and human NPM2 (Thr127 and Ser191). Although other phosphorylation sites are likely important, a casein kinase II-NPM2 interaction in vivo could be predicted in mammals.

The present invention provides three novel proteins, O1-180 (SEQ.ID.NO.2, SEQ.ID.NO.16), O1-184 (SEQ.ID.NO.4), O1-236 (SEQ.ID.NO.6, SEQ.ID.NO.9), the polynucleotide sequences that encode them, and fragments and derivatives thereof. Expression of O1-180, O1-184, O1-236 is highly tissue-specific, being expressed in cells primarily in ovarian tissue. In one embodiment, the invention provides a method for detection of a cell proliferative or degenerative disorder of the ovary, which is associated with expression of O1-180, O1-184 or O1-236. In another embodiment, the invention provides a method for treating a cell proliferative or degenerative disorder associated with abnormal expression of O1-O1-180, O1-184, O1-236 by using an agent which suppresses or enhances their respective activities.

Based on the known activities of many other ovary specific proteins, it can be expected that O1-180, O1-184 and O1-236, as well as fragments and derivatives thereof, will also possess biological activities that will make them useful as diagnostic and therapeutic reagents.

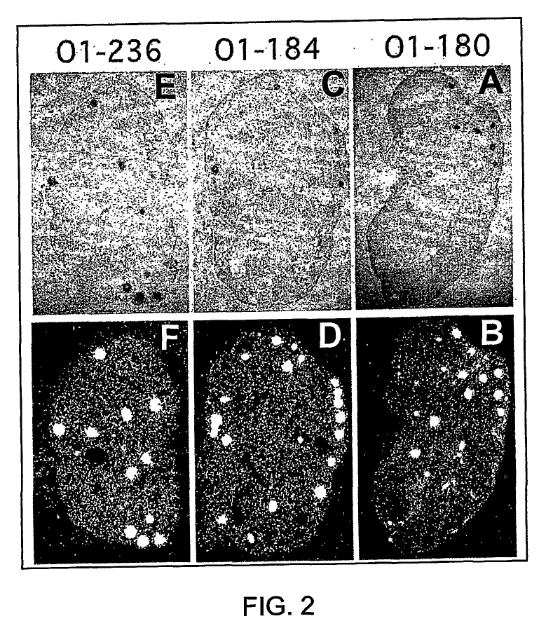

For example, GDF-9 is an oocyte-expressed gene product which has a similar pattern of expression as O1-180, O1-184, and O1-236. It has been shown that mice lacking GDF-9 are infertile at a very early stage of follicular development, at the one-layer primary follicle stage (Dong, et al.). These studies demonstrate that agents which block GDF-9 function would be useful as contraceptive agents in human females. Since O1-180, O1-184, and O1-236 have an expression pattern in the oocyte (FIG. 2) which is nearly identical to GDF-9, this suggests that mice and humans or any other mammal lacking any of all of these gene products would also be infertile. Thus, blocking the function of any or all of these gene products would result in a contraceptive action.

Another regulatory protein that has been found to have ovary-specific expression is inhibin, a specific and potent polypeptide inhibitor of the pituitary secretion of FSH. Inhibin has been isolated from ovarian follicular fluid. Because of its suppression of FSH, inhibin has been advanced as a potential contraceptive in both males and females. O1-180, O1-184 and O1-236 may possess similar biological activity since they are also ovarian specific peptides. Inhibin has also been shown to be useful as a marker for certain ovarian tumors (Lappohn et al., 1989). O1-180, O1-184, O1-236 may also be useful as markers for identifying primary and metastatic neoplasms of ovarian origin. Likewise, mice which lack inhibin develop granulosa cell tumors (Matuk et al., 1992). Similarly, O1-180, O1-184 and O1-236 may be useful as indicators of developmental or reproductive anomalies in prenatal screening procedures.

Mullerian inhibiting substance (MIS or anti-Mullerian hormone) peptide, which is produced by the testis and is responsible for the regression of the Mullerian ducts in the male embryo, has been shown to inhibit the growth of human ovarian cancer in nude mice (Donahoe et al., 1981). O1-180, O1-184 and O1-236 may function similarly and may, therefore, be targets for anti-cancer agents, such as for the treatment of ovarian cancer.

O1-180, O1-184 and O1-236 proteins, agonists and antagonists thereof can be used to identify agents which inhibit fertility (e.g., act as a contraceptive) in a mammal (e.g., human). Additionally, O1-180, O1-184 and O1-236 and agonists and antagonists thereof can be used to identify agents which enhance fertility (e.g., increase the success of in vivo or in vitro fertilization) in a mammal. Likewise, assays of these or related oocyte-expressed gene products can be used in diagnostic assays for detecting forms of infertility (e.g., in an assay to analyze activity of these gene products) or other diseases (e.g., germ cell tumors, polycystic ovary syndrome). Yet further, these proteins or agents which act on these pathways may also function as growth stimulatory factors and, therefore, be useful for the survival of various cell populations in vitro. In particular, if O1-180, O1-184 and/or O1-236 play a role in oocyte maturation, they may be useful targets for in vitro fertilization procedures, e.g., in enhancing the success rate.

A. Proteins

The present invention relates to O1-180 (SEQ.ID.NO.2, SEQ.ID.NO.16), O1-184 (SEQ.ID.NO.4) and O1-236 (SEQ.ID. NO 6, SEQ.ID.NO.9) polypeptides, proteins, or agents thereof.

In addition to the entire O1-180, O1-184 or O1-236 molecules, the present invention also relates to fragments of the polypeptides that may or may not retain the functions described below. Fragments, including the N-terminus of the molecule, may be generated by genetic engineering of translation stop sites within the coding region. Alternatively, treatment of the O1-180, O1-184 or O1-236 with proteolytic enzymes, known as proteases, can produce a variety of N-terminal, C-terminal and internal fragments. Fragments of proteins are seen to include any peptide that contains 6 contiguous amino acids or more that are identical to 6 contiguous amino acids of sequences of SEQ.ID.NO. 2, SEQ.ID.NO. 4, SEQ.ID.NO. 6, SEQ.ID.NO. 9 and SEQ.ID.NO.16. Fragments that contain 7, 8, 9, 10, 11, 12, 13, 14 15, 16, 17, 18, 19, 20, 21, 22, 23, 24, 25, 30, 35, 40, 45, 50, 55, 60, 65, 75, 80, 85, 90, 95, 100, 200 or more contiguous amino acids or more that are identical to a corresponding number of amino acids of any of the sequences of SEQ.ID.NO. 2, SEQ.ID.NO. 4, SEQ.ID.NO. 6, SEQ.ID.NO. 9 and SEQ.ID.NO.16 are also contemplated. Fragments may be used to generate antibodies. Particularly useful fragments will be those that make up domains of O1-180, O1-184 or O1-236. Domains are defined as portions of the proteins having a discrete tertiary structure and that is maintained in the absence of the remainder of the protein. Such structures can be found by techniques known to those skilled in the art. The protein is partially digested with a protease such as subtilisin, trypsin, chymotrypsin or the like and then subjected to polyacrylamide gel electrophoresis to separate the protein fragments. The fragments can then be transferred to a PVDF membrane and subjected to micro sequencing to determine the amino acid sequence of the N-terminal of the fragments.

The term substantially pure as used herein refers to O1-180, O1-184 and O1-236 which are substantially free of other proteins, lipids, carbohydrates or other materials with which they are naturally associated. One skilled in the art can purify O1-180, O1-184 and O1-236 using standard techniques for protein purification. The substantially pure polypeptide will yield a single major band on a non-reducing polyacrylamide gel. The purity of the O1-180, O1-184 and O1-236 polypeptides can also be determined by amino-terminal amino acid sequence analysis. O1-180, O1-184 and O1-236 polypeptides include functional fragments of the polypeptides, as long as their activities remain. Smaller peptides containing the biological activities of O1-180, O1-184 and O1-236.

The polypeptides of the invention include the disclosed sequences and conservative variations thereof The term conservative variation as used herein denotes the replacement of an amino acid residue by another, biologically similar residue. Examples of conservative variations include the substitution of one hydrophobic residue such as isoleucine, valine, leucine or methionine for another, or the substitution of one polar residue for another, such as the substitution of arginine for lysine, glutamic acid for aspartic acid, or glutamine for asparagine, and the like. The term “conservative variation” also includes the use of a substituted amino acid in place of an unsubstituted parent amino acid provided that antibodies raised to the substituted polypeptide also immunoreact with the unsubstituted polypep-tide.

Minor modifications of the recombinant O1-180, O1-184 and O1-236 primary amino acid sequences may result in proteins which have substantially equivalent activity as compared to the respective O1-180, O1-184 and O1-236 polypeptides described herein. Such modifications may be deliberate, as by site-directed mutagenesis, or may be spontaneous. All of the polypeptides produced by these modifications are included herein as long as the biological activity of O1-180, O1-184 or O1-236 still exists. Further, deletion of one or more amino acids can also result in a modification of the structure of the resultant molecule without significantly altering its biological activity. This can lead to the development of a smaller active molecule which would have broader utility. For example, one could remove amino or carboxy terminal amino acids which may not be required for biological activity of O1-180, O1-184 or O1-236.

For the purpose of this invention, the term derivative shall mean any molecules which are within the skill of the ordinary practitioner to make and use, which are made by modifying the subject compound, and which do not destroy the activity of the derivatized compound. Compounds which meet the foregoing criteria which diminish, but do not destroy, the activity of the derivatized compound are considered to be within the scope of the term derivative. Thus, according to the invention, a derivative of a compound comprising amino acids in a sequence corresponding to the sequence of O1-180, O1-184 or O1-236, need not comprise a sequence of amino acids that corresponds exactly to the sequence of O1-180, O1-184 or O1-236, so long as it retains a measurable amount of the activity of the O1-180, O1-184 or O1-236.

Equally, the same considerations may be employed to create a protein, polypeptide or peptide with countervailing, e.g., antagonistic properties. This is relevant to the present invention in which O1-180, O1-184 or O1-236 mutants or analogues may be generated. For example, a O1-180, O1-184 or O1-236 mutant may be generated and tested for O1-180, O1-184 or O1-236 activity to identify those residues important for O1-180, O1-184 or O1-236 activity. O1-180, O1-184 or O1-236 mutants may also be synthesized to reflect a O1-180, O1-184 or O1-236 mutant that occurs in the human population and that is linked to the development of cancer. Also, O1-180, O1-184 or O1-236 mutants may be used as antagonists to inhibit or enhance fertitlity. Thus, O1-180, O1-184 or O1-236 mutants may be used as potential contraceptive compositions and/or fertility enhancement compostions.

B. Nucleic Acids

The invention also provides polynucleotides encoding the O1-180 (SEQ.ID.NO.1, SEQ.ID.NO.11, SEQ.ID.NO.13 and SEQ.ID.NO.12), O1-184 (SEQ.ID.NO.3) or O1-236 (SEQ.ID.NO.5, SEQ.ID.NO.7, SEQ.ID.NO.8; SEQ.ID.NO.10, and SEQ.ID.NO.14) proteins and fragments and derivatives thereof These polynucleotides include DNA, cDNA and RNA sequences which encode O1-180, O1-184 or O1-236. It is understood that all polynucleotides encoding all or a portion of O1-180, O1-184 and/or O1-236 are also included herein, as long as they encode a polypeptide with the activity of O1-180 (SEQ.ID.NO.1, SEQ.ID.NO.11, SEQ.ID.NO.13 and SEQ.ID.NO.12), O1-184 (SEQ.ID.NO.3) or O1-236 (SEQ.ID.NO.5, SEQ.ID.NO.7, SEQ.ID.NO.8; SEQ.ID.NO.10, and SEQ.ID.NO.14). Such polynucleotides include naturally occurring, synthetic, and intentionally manipulated polynucleotides. For example, polynucleotides of O1-180 (SEQ.ID.NO.1, SEQ.ID.NO.11, SEQ.ID.NO.13 and SEQ.ID.NO.12), O1-184 (SEQ.ID.NO.3) or O1-236 (SEQ.ID.NO.5, SEQ.ID.NO.7, SEQ.ID.NO.8; SEQ.ID.NO.10, and SEQ.ID.NO.14) may be subjected to site-directed mutagenesis. The polynucleotide sequences for O1-180, O1-184 and O1-236 also includes antisense sequences. The polynucleotides of the invention include sequences that are degenerate as a result of the genetic code. There are 20 natural amino acids, most of which are specified by more than one codon. Therefore, all degenerate nucleotide sequences are included in the invention as long as the amino acid sequences of O1-180, O1-184 and O1-236 polypeptides encoded by the nucleotide sequences are functionally unchanged.

DNA sequences of the invention can be obtained by several methods. For example, the DNA can be isolated using hybridization or amplification techniques which are well known in the art. These include, but are not limited to: 1) hybridization of genomic or cDNA libraries with probes to detect homologous nucleotide sequences, 2) antibody screening of expression libraries to detect cloned DNA fragments with shared structural features, or 3) use of oligonucleotides related to these sequences and the technique of the polymerase chain reaction.

Preferably the O1-180, O1-184 and O1-236 polynucleotides of the invention are derived from a mammalian organism, and most preferably from a mouse, rat, elephant, pig, cow or human. Screening procedures which rely on nucleic acid hybridization make it possible to isolate any gene sequence from any organism, provided the appropriate probe is available. Oligonucleotide probes, which correspond to a part of the sequence encoding the protein in question, can be synthesized chemically. This requires that short, oligopeptide stretches of amino acid sequence must be known. The DNA sequence encoding the protein can be deduced from the genetic code, however, the degeneracy of the code must be taken into account. It is possible to perform a mixed addition reaction when the sequence is degenerate. This includes a heterogeneous mixture of denatured double-stranded DNA. For such screening, hybridization is preferably performed on either single-stranded DNA or denatured double-stranded DNA. Hybridization is particularly useful in the detection of cDNA clones derived from sources where an extremely low amount of mRNA sequences relating to the polypeptide of interest are present. In other words, by using stringent hybridization conditions directed to avoid non-specific binding, it is possible, for example, to allow the autoradiographic visualization of a specific cDNA done by the hybridization of the target DNA to that single probe in the mixture which is its complete complement (Wallace et al., 1981).

The development of specific DNA sequences encoding O1-180, O1-184 and O1-236 can also be obtained by: 1) isolation of double-stranded DNA sequences from the genomic DNA; 2) chemical manufacture of a DNA sequence to provide the necessary codons for the polypeptides of interest; and 3) in vitro synthesis of a double-stranded DNA sequence by reverse transcription of mRNA isolated from a eukaryotic donor cell. In the latter case, a double-stranded DNA complement of mRNA is eventually formed which is generally referred to as cDNA.

Of the three above-noted methods for developing specific DNA sequences for use in recombinant procedures, the isolation of genomic DNA isolates is the least common. This is especially true when it is desirable to obtain the microbial expression of mammalian polypeptides due to the presence of introns.

The synthesis of DNA sequences is frequently the method of choice when the entire sequence of amino acid residues of the desired polypeptide product is known. When the entire sequence of amino acid residues of the desired polypeptides is not known, the direct synthesis of DNA sequences is not possible and the method of choice is the synthesis of cDNA sequences. Among the standard procedures for isolating cDNA sequences of interest is the formation of plasmid- or phage-carrying cDNA libraries, which are derived from reverse transcription of mRNA which is abundant in donor cells that have a high level of genetic expression. When used in combination with polymerase chain reaction technology, even rare expression products can be cloned. In those cases where significant portions of the amino acid sequence of the polypeptide are known, the production of labeled single or double-stranded DNA or RNA probe sequences duplicating a sequence putatively present in the target cDNA may be employed in DNA/DNA hybridization procedures which are carried out on cloned copies of the cDNA which have been denatured into a single-stranded form (Jay et al., 1983).

A cDNA expression library, such as lambda gt11, can be screened indirectly for O1-180, O1-184 and/or O1-236 peptides having at least one epitope, using antibodies specific for O1-180, O1-184 and/or O1-236. Such antibodies can be either polyclonally or monoclonally derived and used to detect expression product indicative of the presence of O1-180, O1-184 and/or O1-236 cDNA.

DNA sequences encoding O1-180, O1-184 or O1-236 can be expressed in vitro by DNA transfer into a suitable host cell. Host cells are cells in which a vector can be propagated and its DNA expressed. The term also includes any progeny of the subject host cell. It is understood that all progeny may not be identical to the parental cell since there may be mutations that occur during replication. However, such progeny are included when the term host cell is used. Methods of stable transfer, meaning that the foreign DNA is continuously maintained in the host, are known in the art.

In the present invention, the O1-180, O1-184 and/or O1-236 polynucleotide sequences may be inserted into a recombinant expression vector. The term recombinant expression vectors refers to a plasmid, virus or other vehicle known in the art that has been manipulated by insertion or incorporation of the O1-180, O1-184 or O1-236 genetic sequences. Such expression vectors contain a promoter sequence which facilitates the efficient transcription of the inserted genetic sequence of the host. The expression vector typically contains an origin of replication, a promoter, as well as specific genes which allow phenotypic selection of the transformed cells. Vectors suitable for use in the present invention include, but are not limited to the T7-based expression vector for expression in bacteria Rosenberg et al., 1987), the pMSXND expression vector for expression in mammalian cells (Lee and Nathans, 1988) and baculovirus-derived vectors for expression in insect cells. The DNA segment can be present in the vector operably linked to regulatory elements, for example, a promoter (e.g. T7, metallothionein 1, or polyhedrin promoters). Polynucleotide sequences encoding O1-180, O1-184 or O1-236 can be expressed in either prokaryotes or eukaryotes. Hosts can include microbial, yeast, insect and mammalian organisms. Methods of expressing DNA sequences having eukaryotic or viral sequences in prokaryotes are well known in the art. Biologically functional viral and plasmid DNA vectors capable of expression and replication in a host are known in the art. Such vectors are used to incorporate DNA sequences of the invention.

Transformation of a host cell with recombinant DNA may be carried out by conventional techniques as are well known to those skilled in the art. Where the host is prokaryotic, such as E coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired.

When the host is a eukaryote, such methods of transfection of DNA as calcium phosphate co-precipitates, conventional mechanical procedures such as microinjection, electroporation, insertion of a plasmid encased in liposomes, or virus vectors may be used. Eukaryotic cells can also be co-transformed with DNA sequences encoding the O1-180, O1-184 or O1-236 cDNA sequences of the invention, and a second foreign DNA molecule encoding a selectable phenotype, such as the neomycin resistance gene. Another method is to use a eukaryotic viral vector, such as simian virus 40 (SV40) or bovine papilloma virus, to transiently infect or transform eukaryotic cells and express the protein. (see for example, Eukaryotic Viral Vectors, Cold Spring Harbor Laboratory, Gluzman ed., 1982).

Isolation and purification of microbial expressed polypeptide, or fragments thereof, provided by the invention, may be carried out by conventional means induding preparative chromatography and immunological separations involving monoclonal or polyclonal antibodies.

The invention includes antibodies immunoreactive with O1-180, O1-184 or O1-236 polypeptides or functional fragments thereof. Antibodies, which consists essentially of pooled monoclonal antibodies with different epitopic specificities, as well as distinct monoclonal antibodies, are provided. Monoclonal antibodies are made from antigen containing fragments of the protein by methods well known to those skilled in the art (Kohler, et al., Nature, 256:495, 1975). The term antibody as used in this invention is meant to include intact molecules as well as fragments thereof, such as Fab and F(ab′)2, which are capable of binding an epitopic determinant on O1-180, O1-184 or O1-236.

C. Diagnositic Uses

The term cell-degenerative disorder denotes the loss of any type of cell in the ovary, either directly or indirectly. For example, in the absence of GDF-9, there is a block in the growth of the granulosa cells leading to eventual degeneration (i.e., death) of the oocytes (Dong et al., 1996). This death of the oocyte appears to lead to differentiation of the granulosa cells. In addition, in the absence of GDF-9, no normal thecal cell layer is formed around the follicles. Thus, in the absence of one oocyte-specific protein, GDF-9, there are defects in three different cell lineages, oocytes, granulosa cells, and thecal cells. In a similar way, death or differentiation of these various cell lineages could be affected by absence or misexpression of O1-180, O1-184, or O1-236.

Absence or misexpression of O1-180, O1-184, or O1-236 could result in defects in the oocyte/egg leading to the inability of the egg to be fertilized by spermatozoa. Alternatively, embryos may not develop or halt development during the early stage of embryogenesis or show defects in fertilization secondary to absence of these oocyte derived factors. Therefore, O1-180, O1-184 or O1-236 compositions may be employed as a diagnostic or prognostic indicator of infertility in general. More specifically, point mutations, deletions, insertions or regulatory perturbations can be identified. The present invention contemplates further the diagnosis of infertility detecting changes in the levels of O1-180, O1-184 or O1-236 expression.

One embodiment of the instant invention comprises a method for detecting variation in the expression of O1-180, O1-184 or O1-236. This may comprise determining the level of O1-180, O1-184 or O1-236 expressed, or determining specific alterations in the expressed product. In specific embodiments, alterations are detected in the expression of O1-180, O1-184 or O1-236.

The biological sample can be tissue or fluid. Various embodiments include cells from the testes and ovaries. Other embodiments include fluid samples such as vaginal fluid or seminal fluid.

Nucleic acids used are isolated from cells contained in the biological sample, according to standard methodologies (Sambrook et al., 1989). The nucleic acid may be genomic DNA or fractionated or whole cell RNA. Where RNA is used, it may be desired to convert the RNA to a complementary DNA (cDNA). In one embodiment, the RNA is whole cell RNA; in another, it is poly-A RNA. Normally, the nucleic acid is amplified.

Depending on the format, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994).

Following detection, one may compare the results seen in a given patient with a statistically significant reference group of normal patients and patients that have been diagnosed with infertility.

It is contemplated that other mutations in the O1-180, O1-184 or O1-236 polynucleotide sequences may be identified in accordance with the present invention by detecting a nucleotide change in particular nucleic acids (U.S. Pat. No. 4,988,617, incorporated herein by reference). A variety of different assays are contemplated in this regard, including but not limited to, fluorescent in situ hybridization (FISH; U.S. Pat. No. 5,633,365 and U.S. Pat. No. 5,665,549, each incorporated herein by reference), direct DNA sequencing, PFGE analysis, Southern or Northern blotting, single-stranded conformation analysis (SSCA), RNAse protection assay, allele-specific oligonucleotide (ASO, e.g., U.S. Pat. No. 5,639,611), dot blot analysis, denaturing gradient gel electrophoresis (e.g., U.S. Pat. No. 5,190,856 incorporated herein by reference), RFLP (e.g., U.S. Pat. No. 5,324,631 incorporated herein by reference) and PCR™-SSCP. Methods for detecting and quantitating gene sequences, such as mutated genes and oncogenes, in for example biological fluids are described in U.S. Pat. No. 5,496,699, incorporated herein by reference.

Yet further, it is contemplated by that chip-based DNA technologies such as those described by Hacia et al. (1996) and Shoemaker et al. (1996) can be used for diagnosis of infertility. Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization. See also Pease et al., (1994); Fodor et al., (1991).

Antibodies can be used in characterizing the O1-180, O1-184 or O1-236 content through techniques such as ELISAs and Western blot analysis. This may provide a prenatal screen or in counseling for those individuals seeking to have children.

The steps of various other useful immunodetection methods have been described in the scientific literature, such as, e.g., Nakamura et al., (1987). Immunoassays, in their most simple and direct sense, are binding assays. Certain preferred immunoassays are the various types of radioimmunoassays (RIA) and immunobead capture assay. Immunohistochemical detection using tissue sections also is particularly useful. However, it will be readily appreciated that detection is not limited to such techniques, and Western blotting, dot blotting, FACS analyses, and the like also may be used in connection with the present invention.

The antibodies of the invention can be bound to many different carriers and used to detect the presence of an antigen comprising the polypeptide of the invention. Samples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding antibodies, or will be able to ascertain such, using routine experimentation.

There are many different labels and methods of labeling known to those of ordinary skill in the art. Examples of the types of labels which can be used in the present invention include enzymes, radioisotopes, fluorescent compounds, colloidal metals, chemiluminescent compounds, phosphorescent compounds, and bioluminescent compounds. Those of ordinary skill in the art will know of other suitable labels for binding to the antibody, or will be able to ascertain such, using routine experimentation.

Another technique which may also result in greater sensitivity consists of coupling the antibodies to low molecular weight haptens. These haptens can then be specifically detected by means of a second reaction. For example, it is common to use such haptens as biotin, which reacts with avidin, or dinitrophenyl, puridoxal, and fluorescein, which can react with specific anti-hapten antibodies.

In using the monoclonal antibodies of the invention for the in vivo detection of antigen, the detectably labeled antibody is given a dose which is diagnostically effective. The term diagnostically effective means that the amount of detectably labeled monoclonal antibody is administered in sufficient quantity to enable detection of the site having the antigen composing a polypeptide of the invention for which the monoclonal antibodies are specific. The concentration of detectably labeled monoclonal antibody which is administered should be sufficient such that the binding to those cells having the polypeptide is detectable compared to the background. Further, it is desirable that the detectably labeled monoclonal antibody be rapidly cleared from the circulatory system in order to give the best target-to-background signal ratio. As a rule, the dosage of detectably labeled monoclonal antibody for in vivo diagnosis will vary depending on such factors as age, sex, and extent of disease of the individual. Such dosages may vary, for example, depending on whether multiple injections are given, antigenic burden, and other factors known to those of skill in the art.

For in vivo diagnostic imaging, the type of detection instrument available is a major factor in selecting a given radioisotope. The radioisotope chosen must have a type of decay which is detectable for a given type of instrument. Still another important factor in selecting a radioisotope for in vivo diagnosis is that deleterious radiation with respect to the host is minimized. Ideally, a radioisotope used for in vivo imaging will lack a particle emission, but produce a large number of photons in the 140-250 keV range, which may readily be detected by conventional gamma cameras.

For in vivo diagnosis, radioisotopes may be bound to immunoglobulin either directly or indirectly by using an intermediate functional group. Intermediate functional groups which often are used to bind radioisotopes which exist as metallic ions to immunoglobulins are the bifunctional chelating agents such as diethylenetriaminepentacetic acid (DTPA) and ethylenediaminetetraacetic acid (EDTA) and similar molecules. Typical examples of metallic ions which can be bound to the monoclonal antibodies of the invention are ¹¹¹In, ⁹⁷Ru, ⁶⁷Ga, ⁶⁸Ga, ⁷²As, ⁸⁹Zr and ²⁰¹Ti.

The monoclonal antibodies of the invention can also be labeled with a paramagnetic isotope for purposes of in vivo diagnosis, as in magnetic resonance imaging (MRI) or electron spin resonance (ESR). In general, any conventional method for visualizing diagnostic imaging can be utilized. Usually gamma and positron emitting radioisotopes are used for camera imaging and paramagnetic isotopes for MRI. Elements which are particularly useful in such techniques include ¹⁵⁷Gd, 55Mn, ¹⁶²Dy, ⁵⁵Cr and ⁵⁶Fe.

The term cell-proliferative disorder or hyperproliferative disorder denotes malignant as well as non-malignant cell populations which often appear to differ from the surrounding tissue both morphologically and genotypically. The O1-180, O1-184 and O1-236 polynucleotides that are antisense molecules are useful in treating malignancies of the various organ systems, particularly, for example, the ovaries. Essentially, any disorder which is etiologically linked to altered expression of O1-180, O1-184 or O1-236 could be considered susceptible to treatment with a O1-180, O1-184 or O1-236 suppressing reagent, respectively.

The invention provides a method for detecting a cell proliferative disorder of the ovary which comprises contacting an anti-O1-180, O1-184 or O1-236 antibody with a cell suspected of having an O1-180, O1-184 or O1-236 associated disorder and detecting binding to the antibody. The antibody reactive with O1-180, O1-184 or O1-236 is labeled with a compound which allows detection of binding to O1-180, O1-184 or O1-236, respectively. For purposes of the invention, an antibody specific for an O1-180, O1-184 or O1-236 polypeptide may be used to detect the level of O1-180, O1-184 or O1-236, respectively, in biological fluids and tissues. Any specimen containing a detectable amount of antigen can be used. A preferred sample of this invention is tissue of ovarian origin, specifically tissue containing oocytes or ovarian follicular fluid. The level of O1-180, O1-184 or O1-236 in the suspect cell can be compared with the level in a normal cell to determine whether the subject has an O1-180, O1-184 or O1-236-associated cell proliferative disorder. Preferably the subject is human. The antibodies of the invention can be used in any subject in which, it is desirable to administer in vitro or in vivo immunodiagnosis or immunotherapy. The antibodies of the invention are suited for use, for example, in immuno assays in which they can be utilized in liquid phase or bound to a solid phase carrier. In addition, the antibodies in these immunoassays can be detectably labeled in various ways. Examples of types of immunoassays which can utilize antibodies of the invention are competitive and non-competitive immunoassays in either a direct or indirect format. Examples of such immunoassays are the radioimmunoassay (RIA) and the sandwich (ELISA) assay. Detection of the antigens using the antibodies of the invention can be done utilizing immunoassays which are run in either the forward, reverse, or simultaneous modes, including immunohistochemical assays on physiological samples. Those of skill in the art will know, or can readily discern, other immunoassay formats without undue experimentation.

D. Therapeutic Uses

Due to the expression of O1-180, O1-184 and O1-236 in the reproductive tract, there are a variety of applications using the polypeptides, polynucleotides and antibodies of the invention, related to contraception, fertility and pregnancy. O1-180, O1-184 and O1-236 could play a role in regulation of the menstrual cycle and, therefore, could be useful in various contraceptive regimens.

It is also contemplated that O1-180, O1-184, or O1-236 polynucleotide sequences, polypeptide sequences, antibodies, fragments thereof or mutants thereof may be used to inhibit or enhance early embryogenesis by distrubing the maternal genome. One of skill in the art is aware that disruptions of the maternal genome that cause phenotypes in embryonic development are termed maternal effect mutations. Two such examples have been characterized in mice using knockout technology. In each example, the gene product is normally accumulated in growing oocytes and persists in the early developing embryo and the phenotype affects offspring of knockout females, regardless of their genotype or gender. The first identified gene encodes MATER (maternal antigen that embryos require), which is necessary for development beyond the two-cell stage and has been implicated in establishing embryonic genome transcription patterns (Tong et al., 2000). The second identified gene encodes DNMT1o, an oocyte-specific DNA methyltransferase critical for maintaining imprinting patterns established in the embryonic genome and the viability of the developing mouse during the last third of gestation (Howell et al., 2001). Presumably many other oocyte-derived factors mediate the complexities of early embryogenesis, thus, it is contemplated that the O1-180 and O1-236 are maternal effect genes since they function in processes of early embryogenesis.

In further embodiments, it is contemplated that O1-236 or NPM2 may play a role in in chromatin remodeling during early embryoonic development. For example, studies have predicted the presence of a mammalian nuclear protein that is necessary for oocyte remodeling of sperm DNA, and is released into the ooplasm at germinal vesicle breakdown (Maeda et al., 1998). Yet further, it is known that oocytes can efficiently remodel not only sperm nuclei during fertilization, but also somatic cell nuclei. Thus, the inventors have contemplated the role of NPM2 in nuclear transfer cloning (Zuccotti et al., 2000). It envisioned that NPM2 (encoded by O1-236) is a critical factor in mammalian oocytes for chromatin remodeling during early embryonic development. Thus, supplementing enucleated oocytes with NPM2 may facilitate cloning by nuclear transfer technologies.

The monoclonal antibodies of the invention can be used in vitro and in vivo to monitor the course of amelioration of an O1-180, O1-184 or O1-236-associated disease in a subject. Thus, for example, by measuring the increase or decrease in the number of cells expressing antigen comprising a polypeptide of the invention or changes in the concentration of such antigen present in various body fluids, it would be possible to determine whether a particular therapeutic regimen aimed at ameliorating the O1-180, O1-184 or O1-236-associated disease is effective. The term ameliorate denotes a lessening of the detrimental effect of the O1-180, O1-184 or O1-236-associated disease in the subject receiving therapy.

The present invention identifies nucleotide sequences that can be expressed in an altered manner as compared to expression in a normal cell, therefore, it is possible to design appropriate therapeutic or diagnostic techniques directed to this sequence. Thus, where a cell-proliferative disorder is associated with the expression of O1-180, O1-184 or O1-236, nucleic acid sequences that interfere with the expression of O1-180, O1-184 or O1-236, respectively, at the translational level can be used. This approach utilizes, for example, antisense nucleic acids or ribozymes to block translation of a specific O1-180, O1-184 or O1-236 mRNA, either by masking that mRNA with an antisense nucleic acid or by cleaving it with a ribozyme.

Antisense nucleic acids are DNA or RNA molecules that are complementary to at least a portion of a specific mRNA molecule (Weintraub, 1990). In the cell, the antisense nucleic acids hybridize to the corresponding mRNA, forming a double-stranded molecule. The antisense nucleic acids interfere with the translation of the mRNA, since the cell will not translate a mRNA that is double-stranded. Antisense oligomers of about 15 nucleotides are preferred, since they are easily synthesized and are less likely to cause problems than larger molecules when introduced into the target O1-180, O1-184 or O1-236-producing cell. The use of antisense methods to inhibit the in vitro translation of genes is well known in the art (Marcus-Sakura, 1988).

Ribozymes are RNA molecules possessing the ability to specifically cleave other single-stranded RNA in a manner analogous to DNA restriction endonucleases. Through the modification of nucleotide sequences which encode these RNAs, it is possible to engineer molecules that recognize specific nucleotide sequences in an RNA molecule and cleave it (Cech, 1988). A major advantage of this approach is that, because they are sequence-specific, only mRNAs with particular sequences are inactivated.

There are two basic types of ribozymes namely, tetrahymena-type (Hasselhoff, 1988) and “hammerhead”-type. Tetrahymena-type ribozymes recognize sequences which are four bases in length, while “hammerhead”-type ribozymes recognize base sequences 11-18 bases in length. The longer the recognition sequence, the greater the likelihood that the sequence will occur exclusively in the target mRNA species. Consequently, hammerhead-type ribozymes are preferable to tetrahymena-type ribozymes for inactivating a specific mRNA species and 18-based recognition sequences are preferable to shorter recognition sequences.

The present invention also provides gene therapy for the treatment of cell proliferative or degenerative disorders which are mediated by O1-180, O1-184 or O1-236 proteins. Such therapy would achieve its therapeutic effect by introduction of the respective O1-180, O1-184 or O1-236 cDNAs or O1-180, O1-184, or O1-236 antisense polynucleotide into cells having the proliferative or degenerative disorder. Delivery of O1-180, O1-184, or O1-236 cDNAs or antisense O1-180, O1-184 or O1-236 polynucleotides can be achieved using a recombinant expression vector such as a chimeric virus or a colloidal dispersion system. Especially preferred for therapeutic delivery of cDNAs or antisense sequences is the use of targeted liposomes.

Various viral vectors which can be utilized for gene therapy as taught herein include adenovirus, herpes virus, vaccinia, or, preferably, an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. By inserting an O1-180, O1-184 or O1-236 sequence of interest into the viral vector, along with another gene which encodes the ligand for a receptor on a specific target cell, for example, the vector is now target specific. Retroviral vectors can be made target specific by inserting, for example, a polynucleotide encoding a sugar, a glycolipid, or a protein. Preferred targeting is accomplished by using an antibody to target the retroviral vector. Those of skill in the art will know of, or can readily ascertain without undue experimentation, specific polynucleotide sequences which can be inserted into the retroviral genome to allow target specific delivery of the retroviral vector containing an O1-180, O1-184 or O1-236 cDNA or O1-180, O1-184, or O1-236 antisense polynucleotides.

Since recombinant retroviruses are defective, they require assistance in order to produce infectious vector particles. This assistance can be provided, for example, by using helper cell lines that contain plasmids encoding all of the structural genes of the retrovirus under the control of regulatory sequences within the LTR. These plasmids are missing a nucleotide sequence which enables the packing mechanism to recognize an RNA transcript for encapsidation. Helper cell lines which have deletions of the packaging signal include, but are not limited to ψ2, PA317 and PA12, for example. These cell lines produce empty virions, since no genome is packaged. If a retroviral vector is introduced into such cells in which the packaging signal is intact, but the structural genes are replaced by other genes of interest, the vector can be packaged and vector virion produced.

Alternatively NIH 3T3 or other tissue culture cells can be directly transfected with plasmids encoding the retroviral structural genes gag, pol and env, by conventional calcium phosphate transfection. These cells are then transfected with the vector plasmid containing the genes of interest. The resulting cells release the retroviral vector into the culture medium.

Another targeted delivery system for O1-180, O1-184 or O1-236 cDNAs or O1-180, O1-184, or O1-236 antisense polynucleotides is a colloidal dispersion system. Colloidal dispersion systems include macromolecule complexes, nanocapsules complexes, nanocapsules, microspheres, beads, and lipid-based systems including oil-in-water emulsions, micelles, mixed micelles, and liposomes. The preferred colloidal system of this invention is a liposome. Liposomes are artificial membrane vesicles which are useful as delivery vehicles in vitro and in vivo. It has been shown that large unilamellar vesicles (LUV), which range in size from 0.2-4.0 μm can encapsulate a substantial percentage of an aqueous buffer containing large macromolecules. RNA, DNA and intact virions can be encapsulated within the aqueous interior and be delivered to cells in a biologically active form (Fraley et al. 1981). In addition to mammalian cells, liposomes have been used for delivery of polynucleotides in plant, yeast and bacterial cells. In order for a liposome to be an efficient gene transfer vehicle, the following characteristics should be present: (1) encapsulation of the genes of interest at high exigency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Manning et al., 1988).

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand.

E. Screening for Modulators

The present invention also contemplates the use of O1-180, O1-184 or O1-236 and active fragments, and nucleic acids coding therefore, in the screening of compounds for activity in either stimulating O1-180, O1-184 or O1-236, overcoming the lack of O1-180, O1-184 or O1-236 or blocking or inhibiting the effect of an O1-180, O1-184 or O1-236 molecule. These assays may make use of a variety of different formats and may depend on the kind of “activity” for which the screen is being conducted.

In one embodiment, the invention is to be applied for the screening of compounds that bind to the O1-180, O1-184 or O1-236 polypeptide or fragment thereof. The polypeptide or fragment may be either free in solution, fixed to a support, expressed in or on the surface of a cell. Either the polypeptide or the compound may be labeled, thereby permitting determining of binding.

In another embodiment, the assay may measure the inhibition of binding of O1-180, O1-184 or O1-236 to a natural or artificial substrate or binding partner. Competitive binding assays can be performed in which one of the agents (O1-180, O1-184 or O1-236, binding partner or compound) is labeled. Usually, the polypeptide will be the labeled species. One may measure the amount of free label versus bound label to determine binding or inhibition of binding.

Another technique for high throughput screening of compounds is described in WO 84/03564. Large numbers of small peptide test compounds are synthesized on a solid substrate, such as plastic pins or some other surface. The peptide test compounds are reacted with O1-180, O1-184 or O1-236 and washed. Bound polypeptide is detected by various methods.

Purified O1-180, O1-184 or O1-236 can be coated directly onto plates for use in the aforementioned drug screening techniques. However, non-neutralizing antibodies to the polypeptide can be used to immobilize the polypeptide to a solid phase. Also, fusion proteins containing a reactive region (preferably a terminal region) may be used to link the O1-180, O1-184 or O1-236 active region to a solid phase.

Various cell lines containing wild-type or natural or engineered mutations in O1-180, O1-184 or O1-236 gene can be used to study various functional attributes of O1-180, O1-184 or O1-236 and how a candidate compound affects these attributes. Methods for engineering mutations are described elsewhere in this document, as are naturally-occurring mutations in O1-180, O1-184 or O1-236 that lead to, contribute to and/or otherwise cause infertility. In such assays, the compound would be formulated appropriately, given its biochemical nature, and contacted with a target cell. Depending on the assay, culture may be required. The cell may then be examined by virtue of a number of different physiologic assays. Alternatively, molecular analysis may be performed in which the function of O1-180, O1-184 or O1-236, or related pathways, may be explored.

In a specific embodiment, yeast two-hybrid analysis is performed by standard means in the art with the polypeptides of the present invention, i.e., O1-180, O1-184 or O1-236. Two hybrid screen is used to elucidate or characterize the function of a protein by identifying other proteins with which it interacts. The protein of unknown function, herein referred to as the “bait” is produced as a chimeric protein additionally containing the DNA binding domain of GAL4. Plasmids containing nucleotide sequences which express this chimeric protein are transformed into yeast cells, which also contain a representative plasmid from a library containing the GAL4 activation domain fused to different nucleotide sequences encoding different potential target proteins. If the bait protein physically interacts with a target protein, the GAL4 activation domain and GAL4 DNA binding domain are tethered and are thereby able to act conjunctively to promote transcription of a reporter gene. If no interaction occurs between the bait protein and the potential target protein in a particular cell, the GAL4 components remain separate and unable to promote reporter gene transcription on their own. One skilled in the art is aware that different reporter genes can be utilized, including β-galactosidase, HIS3, ADE2, or URA3. Furthermore, multiple reporter sequences, each under the control of a different inducible promoter, can be utilized within the same cell to indicate interaction of the GAL4 components (and thus a specific bait and target protein). A skilled artisan is aware that use of multiple reporter sequences decreases the chances of obtaining false positive candidates. Also, alternative DNA-binding domain/activation domain components may be used, such as LexA. One skilled in the art is aware that any activation domain may be paired with any DNA binding domain so long as they are able to generate transactivation of a reporter gene. Furthermore, a skilled artisan is aware that either of the two components may be of prokaryotic origin, as long as the other component is present and they jointly allow transactivation of the reporter gene, as with the LexA system.

Two hybrid experimental reagents and design are well known to those skilled in the art (see The Yeast Two-Hybrid System by P. L. Bartel and S. Fields (eds.) (Oxford University Press, 1997), including the most updated improvements of the system (Fashena et al., 2000). A skilled artisan is aware of commercially available vectors, such as the Matchmaker™ Systems from Clontech (Palo Alto, Calif.) or the HybriZAP® 2.1 Two Hybrid System (Stratagene; La Jolla, Calif.), or vectors available through the research community (Yang et al., 1995; James et al, 1996). In alternative embodiments, organisms other than yeast are used for two hybrid analysis, such as mammals (Mammalian Two Hybrid Assay Kit from Stratagene (La Jolla, Calif.)) or E. coli (Hu et al., 2000).

In an alternative embodiment, a two hybrid system is utilized wherein protein-protein interactions are detected in a cytoplasmic-based assay. In this embodiment, proteins are expressed in the cytoplasm, which allows posttranslational modifications to occur and permits transcriptional activators and inhibitors to be used as bait in the screen. An example of such a system is the CytoTrap® Two-Hybrid System from Stratagene (La Jolla, Calif.), in which a target protein becomes anchored to a cell membrane of a yeast which contains a temperature sensitive mutation in the cdc25 gene, the yeast homologue for hSos (a guanyl nucleotide exchange factor). Upon binding of a bait protein to the target, hSos is localized to the membrane, which allows activation of RAS by promoting GDP/GTP exchange. RAS then activates a signaling cascade which allows growth at 37° C. of a mutant yeast cdc25H. Vectors (such as pMyr and pSos) and other experimental details are available for this system to a skilled artisan through Stratagene (La Jolla, Calif.). (See also, for example, U.S. Pat. No. 5,776,689, herein incorporated by reference).

Thus, in accordance with an embodiment of the present invention, there is a method of screening for a peptide which interacts with O1-180, O1-184 or O1-236 comprising introducing into a cell a first nucleic acid comprising a DNA segment encoding a test peptide, wherein the test peptide is fused to a DNA binding domain, and a second nucleic acid comprising a DNA segment encoding at least part of O1-180, O1-184 or O1-236, respectively, wherein the at least part of O1-180, O1-184 or O1-236 respectively, is fused to a DNA activation domain. Subsequently, there is an assay for interaction between the test peptide and the O1-180, O1-184 or O1-236 polypeptide or fragment thereof by assaying for interaction between the DNA binding domain and the DNA activation domain. For example, the assay for interaction between the DNA binding and activation domains may be activation of expression of β-galactosidase.

An alternative method is screening of lambda.gtl1, lambda.LZAP (Stratagene) or equivalent CDNA expression libraries with recombinant O1-180, O1-184 or O1-236. Recombinant O1-180, O1-184 or O1-236 or fragments thereof are fused to small peptide tags such as FLAG, HSV or GST. The peptide tags can possess convenient phosphorylation sites for a kinase such as heart muscle creatine kinase or they can be biotinylated. Recombinant O1-180, O1-184 or O1-236 can be phosphorylated with ³²[P] or used unlabeled and detected with streptavidin or antibodies against the tags. .lambda.gtl1cDNA expression libraries are made from cells of interest and are incubated with the recombinant O1-180, O1-184 or O1-236, washed and cDNA clones which interact with O1-180, O1-184 or O1-236 isolated. Such methods are routinely used by skilled artisans. See, e.g., Sambrook (supra).

Another method is the screening of a mammalian expression library in which the cDNAs are cloned into a vector between a mammalian promoter and polyadenylation site and transiently transfected in cells. Forty-eight hours later the binding protein is detected by incubation of fixed and washed cells with a labeled O1-180, O1-184 or O1-236. In this manner, pools of cDNAs containing the cDNA encoding the binding protein of interest can be selected and the cDNA of interest can be isolated by further subdivision of each pool followed by cycles of transient transfection, binding and autoradiography. Alternatively, the CDNA of interest can be isolated by transfecting the entire cDNA library into mammalian cells and panning the cells on a dish containing the O1-180, O1-184 or O1-236 bound to the plate. Cells which attach after washing are lysed and the plasmid DNA isolated, amplified in bacteria, and the cycle of transfection and panning repeated until a single cDNA clone is obtained. See Seed et al., 1987 and Aruffo et al., 1987 which are herein incorporated by reference. If the binding protein is secreted, its cDNA can be obtained by a similar pooling strategy once a binding or neutralizing assay has been established for assaying supernatants from transiently transfected cells. General methods for screening supernatants are disclosed in Wong et al., (1985).

Another alternative method is isolation of proteins interacting with the O1-180, O1-184 or O1-236 directly from cells. Fusion proteins of O1-180, O1-184 or O1-236 with GST or small peptide tags are made and immobilized on beads. Biosynthetically labeled or unlabeled protein extracts from the cells of interest are prepared, incubated with the beads and washed with buffer. Proteins interacting with the O1-180, O1-184 or O1-236 are eluted specifically from the beads and analyzed by SDS-PAGE. Binding partner primary amino acid sequence data are obtained by microsequencing. Optionally, the cells can be treated with agents that induce a functional response such as tyrosine phosphorylation of cellular proteins. An example of such an agent would be a growth factor or cytoline such as interleukin-2.

Another alternative method is immunoaffinity purification. Recombinant O1-180, O1-184 or O1-236 is incubated with labeled or unlabeled cell extracts and immunoprecipitated with anti-O1-180, O1-184 or O1-236 antibodies. The immunoprecipitate is recovered with protein A-Sepharose and analyzed by SDS-PAGE. Unlabelled proteins are labeled by biotinylation and detected on SDS gels with streptavidin. Binding partner proteins are analyzed by microsequencing. Further, standard biochemical purification steps known to those skilled in the art may be used prior to microsequencing.

Yet another alternative method is screening of peptide libraries for binding partners. Recombinant tagged or labeled O1-180, O1-184 or O1-236 is used to select peptides from a peptide or phosphopeptide library which interact with the O1-180, O1-184 or O1-236. Sequencing of the peptides leads to identification of consensus peptide sequences which might be found in interacting proteins.

The present invention also encompasses the use of various animal models. Thus, any identity seen between human and other animal O1-180, O1-184 or O1-236 provides an excellent opportunity to examine the function of O1-180, O1-184 or O1-236 in a whole animal system where it is normally expressed. By developing or isolating mutant cells lines that fail to express normal O1-180, O1-184 or O1-236, one can generate models in mice that enable one to study the mechanism of O1-180, O1-184 or O1-236 and its role in oogenesis and embryonic development.

Treatment of animals with test compounds will involve the administration of the compound, in an appropriate form, to the animal. Administration will be by any route that could be utilized for clinical or non-clinical purposes, including but not limited to oral, nasal, buccal, rectal, vaginal or topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood or lymph supply and intratumoral injection.

Determining the effectiveness of a compound in vivo may involve a variety of different criteria. Such criteria include, but are not limited to, increased fertility, decreased fertility or contraception.

In one embodiment of the invention, transgenic animals are produced which contain a functional transgene encoding a functional O1-180, O1-184 or O1-236 polypeptide or variants thereof. Transgenic animals expressing O1-180, O1-184 or O1-236 transgenes, recombinant cell lines derived from such animals and transgenic embryos may be useful in methods for screening for and identifying agents that induce or repress function of O1-180, O1-184 or O1-236. Transgenic animals of the present invention also can be used as models for studying disease states.

In one embodiment of the invention, an O1-180, O1-184 or O1-236 transgene is introduced into a non-human host to produce a transgenic animal expressing an O1-180, O1-184 or O1-236. The transgenic animal is produced by the integration of the transgene into the genome in a manner that permits the expression of the transgene. Methods for producing transgenic animals are generally described by Wagner and Hoppe (U.S. Pat. No. 4,873,191; which is incorporated herein by reference), Brinster et al., 1985; which is incorporated herein by reference in its entirety) and in “Manipulating the Mouse Embryo; A Laboratory Manual” 2nd edition (eds., Hogan, Beddington, Costantimi and Long, Cold Spring Harbor Laboratory Press, 1994; which is incorporated herein by reference in its entirety).

It may be desirable to replace the endogenous O1-180, O1-184 or O1-236 by homologous recombination between the transgene and the endogenous gene; or the endogenous gene may be eliminated by deletion as in the preparation of “knock-out” animals. Typcially, targeting vectors that contain a portion of the gene of interest and a selection marker are generated and transfected into embryonic stem (ES) cells. These targeting vectors are electroporated into the hprt-negative ES cell line and selected in HAT and FIAU. ES cells with the correct mutation are injected into blastocysts to generate chimeras and eventually heterozygotes and homozygotes for the mutant O1-180, O1-184 and O1-236 genes. Thus, the absence of O1-180, O1-184 or O1-236 in “knock-out” mice permits the study of the effects that loss of O1-180, O1-184 or O1-236 protein has on a cell in vivo.

As noted above, transgenic animals and cell lines derived from such animals may find use in certain testing experiments. In this regard, transgenic animals and cell lines capable of expressing wild-type or mutant O1-180, O1-184 or O1-236 may be exposed to test substances. These test substances can be screened for the ability to enhance wild-type O1-180, O1-184 or O1-236 expression and or function or impair the expression or function of mutant O1-180, O1-184 or O1-236.

F. Formulations and Routes for Administration to Patients

Where clinical applications are contemplated, it will be necessary to prepare pharmaceutical compositions—expression vectors, virus stocks, proteins, antibodies and drugs—in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, described supra.

The active compounds also may be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the polypeptides of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient also may be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### G. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Creation of a cDNA Subtractive Hybridization Library

Ovaries from Gdf9 knockout mice are histologically very different from wild-type ovaries due to the early block in folliculogenesis. In particular, one layer primary follicles are relatively enriched in Gdf9 knockout ovaries and abnormal follicular nests are formed after oocyte loss. The inventors took advantage of these differences in ovary composition and related them to alterations in gene expression patterns to clone novel ovary-expressed transcripts which are upregulated in the Gdf9 knockout ovaries.

Ovaries from either Gdf9 knockout mice (C57BL/6/129SvEv hybrid) or wild-type mice were collected and polyA+ mRNA was made from each pool. Using a modified version of the CLONTECH PCR-Select Subtraction kit, the inventors generated a pBluescript SK+plasmid-based cDNA library which was expected to be enriched for sequences upregulated in the Gdf9 knockout ovaries.

Ligations into the NotI site of pBluescript SK+ were performed with a low molar ratio of EagI-digested cDNA fragment inserts to vector to prevent multiple inserts into the vector. Transformations were performed, and >1000 independent bacterial clones were picked and stored in glycerol at −80° C. The remainder of the ligation mix was stored at −80° C. for future transformations.

### Example 2

### Initial Sequence Analysis of pOvary1 (pO1) Library Inserts

The inventors performed sequence analysis of 331 inserts from the pO1 subtractive hybridization of cDNA library. An Applied Biosystems 373 DNA Sequencer was used to sequence these clones. BLAST searches the inventorsre performed using the National Center for Biotechnology Information databases. Novel sequences were analyzed for open reading frames and compared to previously identified novel sequences using DNASTAR analysis programs. A summary of the data is presented in Table 1. As shown, the majority of the clones were known genes or matched mouse or human ESTs. 9.4% of the clones failed to match any known sequence in the database.

TABLE 1
Summary of database searches of pO1 cDNA clones

| pO1 cDNA Matches | Number identified | Percentage |
|---|---|---|
| Known Genes | 180 | 54.4% |
| Mouse/Human EST | 120 | 36.2% |
| RARE ESTs (1 EST match) | (8) | (2.4%) |
| ESTs from 2-cell library | (3) | (0.9%) |
| No match | 31 | 9.4% |
| Total | 331 | 100% |

### Example 3

### Expression Analysis and cDNA Screening of Ovarian-Expressed Genes

Northern blot analysis was performed on all cDNAs which failed to match sequences in any database. Additionally, sequences matching ESTs derived predominantly from mouse 2-cell embryo cDNA libraries (e.g., O1-184, and O1-236) were analyzed. The rationale for analyzing this last group of ESTs was that mRNAs expressed at high levels in oocytes may persist until the 2-cell stage and may play a role in early embryonic development including fertilization of the egg or fusion of the male and female pronuclei.

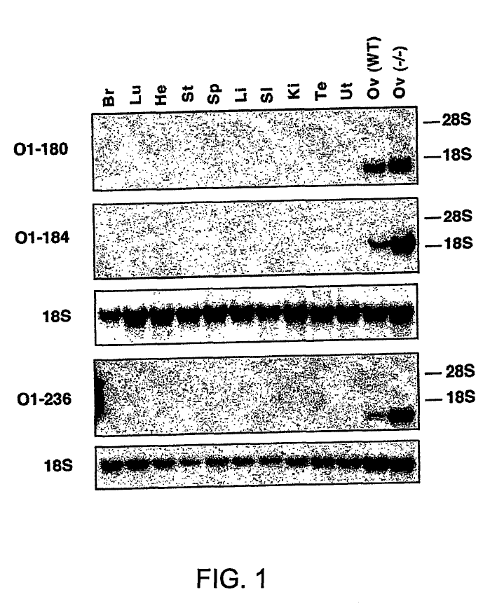

The results of the initial screen of novel ovarian genes is presented in Table 2. Northern blot analysis of 23 clones demonstrated that 8 of these clones were upregulated in the Gdf9 knockout ovary indicating that the subtractive hybridization protocol used was adequate. Northern blot analysis using total RNA isolated from either adult C57BL/6/129SvEv hybrid mice (the ovarian RNA) or Swiss WEBSTER mice (all other tissues) also demonstrated that four of these clones including 2 clones which matched ESTs sequenced from 2-cell libraries were only expressed in the ovary (FIG. 1). The O1-236 fragment probe (749 bp) detected a transcript of approximately 1.0 kb (FIG. 1). Several clones have so far been analyzed for their ovarian localization by in situ hybridization analysis (FIG. 2). Clones O1-180, O1-184, and O1-236 were oocyte-specific and expressed in oocytes of primary (one-layer) preantral follicles through ovulation (FIG. 2).

TABLE 2
Analysis of ovarian cDNAs with no known function

| | PO1 Cdna | | | |
|---|---|---|---|---|
| | Adult | Upregulated in | | Further studies |
| | mRNA | Gdf9 knockout | Database | (in situ hybridization; |
| | Expression | ovary | match | chromosomal mapping) |
| | PO1 Cdna | | | |
| | Adult | Upregulated in | | Further studies |
| | mRNA | GDF-9-deficient | Database | (in situ hybridization; |
| | Expression | ovary | match | chromosomal mapping) |
| 24 | Multiple | No | — | No |
| 27 | Multiple | Yes | — | Oocyte-specific by in situ |
| 37 | Multiple | Yes | — | No |
| 70 | Multiple | No | — | No |
| 91 | | | 1 EST (2-cell) | |
| 97 | Multiple | No | ? | No |
| 101 | Multiple | No1 | — | No |
| 114 | Multiple | No | — | No |
| 110 | Multiple | Yes | — | No |
| 126 | Multiple | Yes | — | No |
| 180 | Ovary-specific | Yes | — | Oocyte-specific by in situ |
| 184 | Ovary-specific | Yes | >1 EST (All 2- | Oocyte-specific by in situ |
| | | | cell) | |
| 186 | Ovary-specific | Yes | — | Granulosa cell-specific by |
| | | | | in situ |
| 223 | Multiple | No | — | No |
| 224 | Multiple | No | — | No |
| 236 | Ovary-specific | Yes | 6 EST (2 c-cell | Oocyte-specific by in situ |
| | | | and others) | |
| 255 | Multiple | No | “zinc-finger” | |
| | | | domains | |
| 279 | Multiple | No | — | No |
| 317 | Multiple | No | — | No |
| 330 | Multiple | No | — | No |
| 331 | Multiple | No | — | No |
| 332 | Multiple | No | — | No |
| 334 | Multiple | No | — | No |
| 371 | Multiple | No | — | No |

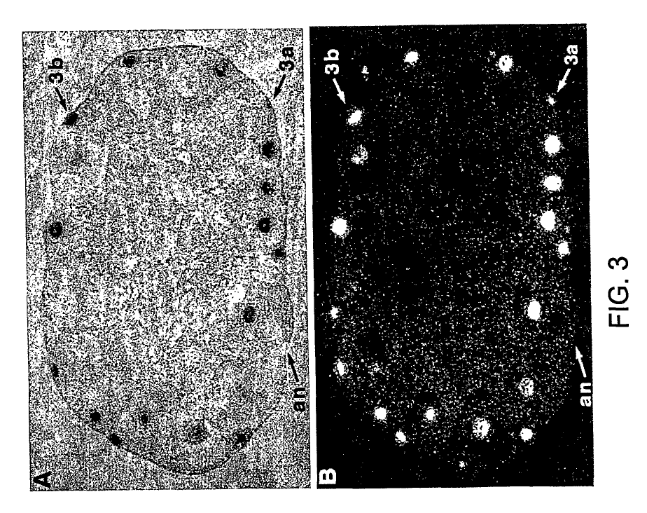

The O1-236 gene product was oocyte-specific (FIG. 3). O1-236 was not expressed in oocytes of primordial (type 2) or small type 3a follicles (Pedersen et al., 1968), but first detected in oocytes of intermediate-size type 3a follicles and all type 3b follicles (i.e., follicles with >20 granulosa cells surrounding the oocyte in largest cross-section). Expression of the O1-236 mRNA persisted through the antral follicle stage. Interestingly, the oocyte-specific. expression pattern of the O1-236 gene product paralleled the expression of other oocyte-specific genes which the inventors have studied including Gdf9 (McGrath et al., 1995) and bone morphogenetic protein 15 (Dube et al., 1998).

### Example 4

### Cloning of Npm2

Wild-type ovary and Gdf9 knockout ZAP Express ovary cDNA libraries were synthesized and were screened to isolate fill-length cDNAs for the above-mentioned three clones. Each fill-length cDNA was again subjected to database searches and analyzed for an open reading frame, initiation ATG, and protein homology. The full-length cDNAs approximate the mRNA sizes determined from Northern blot analysis. Database searches using the predicted amino acid sequence permitted the identification of important domains (e.g., signal peptide sequences, transmembrane domains, zinc fingers, etc.) which are useful to define the possible function and cellular localization of the novel protein.

The O1-236 partial cDNA fragment identified in Example 1 was used to screen Matzuk laboratory ZAP Express (Stratagene) ovarian cDNA libraries generated from either wild-type or GDF-9 deficient ovaries as per manufacturer's instructions and as in Dube et al, 1998). In brief, approximately 300,000 clones of either wild-type or GDF-9 knockout mouse ovary cDNA libraries were hybridized to [alpha-³²P] dCTP random-primed probes in Church's solution at 63° C. Filters were washed with 0.1×Church's solution and exposed overnight at −80° C.

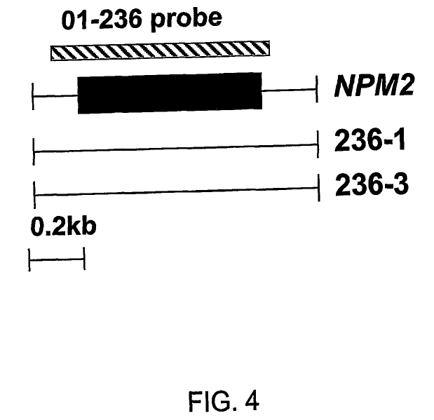

Upon primary screening of the mouse ovarian cDNA libraries, the O1-236 cDNA fragment detected 22 positive phage clones out of 300,000 screened. Two of these clones (236-1 and 236-3), which approximated the mRNA size and which were derived from the two independent libraries, were analyzed further by restriction endonuclease digestion and DNA sequence analysis. These independent clones form a 984 bp overlapping contig (excluding the polyA sequences) and encode a 207 amino acid open reading frame (FIG. 4). Including the polyA tail, this sequence approximates the 1.0 kb mRNA seen by Northern blot analysis suggesting that nearly all of the 5′ UTR sequence had been isolated. When the nucleotide sequence was subjected to public database search, no significant matches were derived. However, database search with the 207 amino acid open reading frame demonstrated high homology with several nucleoplasmin homologs from several species. Interestingly, O1-236 showed highest homology with Xenopus laevis nucleoplasmin. At the amino acid level, O1-236 was 48% identical to Xenopus laevis nucleoplasmin (FIG. 4). Based on this homology and the expression patterns of both gene products in oocytes, the inventors termed the gene Npm2 since it was the mammalian ortholog of Xenopus laevis nucleoplasmin [called Xnpm2 in (MacArthur et al., 1997)]

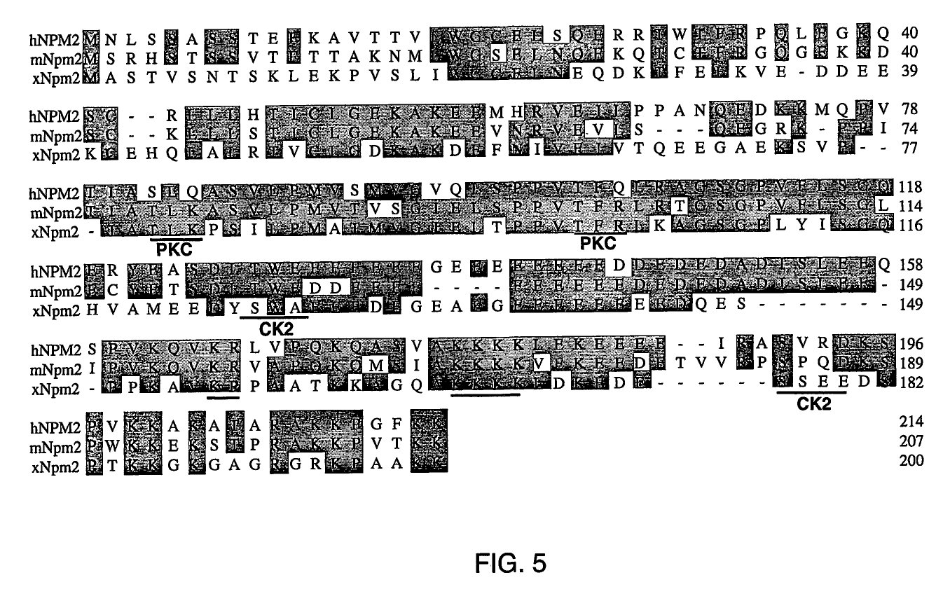

Using the Npm2 cDNA sequence to search the EST database, two human cDNA clones containing sequences homologous to the mouse Npm2 were found. Sequence analysis of these two ESTs was performed. The two independent clones form a 923 bp overlapping contig which encoded a 214 amino acid open reading frame. At the amino acid level, human NPM2 was 48% and 67% identical to Xnpm2 and mouse Npm2, respectably (FIG. 5).

When the frog and mammalian NPM2 sequences were compared, several interesting features were realized. Nucleoplasmin had a bipartite nuclear localization signal consisting of KR-(X)10-KKKK (Dingwall et al., 1987). Deletion of either of these basic amino acid clusters in nucleoplasmin prevented translocation to the nucleus (Robbins et al., 1991). When the mouse and human NPM2 sequences were analyzed, this bipartite sequence was 100% conserved between the two proteins (FIG. 5). Thus, mammalian NPM2 was predicated to translocate to the nucleus where it would primarily function.

Also, conserved between NPM2 and nucleplasmin was a long stretch of negatively charged residues. Amino acids 125-144 of NPM2 and amino acids 128-146 of nucleoplasmin are mostly glutamic acid and aspartic acid residues, with 19 out of the 20 residues for NPM2 and 16 out of the 19 residues for nucleoplasmin either Asp or Glu. This region of Xenopus laevis nucleoplasmin has been implicated to bind the positively charged protamines and histones. Thus, a similar function for this acidic region of NPM2 was predicted.

The last obvious feature of the NPM2 and nucleoplasmin sequences was the high number of serine and threonine residues. The NPM2 sequence contained 19 serine and 17 threonines (i.e., 17.2% of the residues) and nucleoplasmin had 12 serine and 11 threonine residues (i.e., 11.5% of the residues). Multiple putative phosphorylation sites were predicted from the NPM2 and nucleoplasmin sequences. Several putative phosphorylation sequences that were conserved between the two proteins are shown in FIG. 5. Phosphorylation of nucleoplasmin was believed to increase its translocation to the nucleus and also its activity (Sealy et al., 1986, Cotten et al., 1986, Vancurova et al., 1995, Leno et al., 1996). Similarly, phosphorylation may also alter NPM2 activity. Thus, since both mouse and human NPM2 and Xenopus laevis nucleoplasm are oocyte (and egg)-specific at the mRNA level and share highest identity, it was concluded that mammalian NPM2 and frog nucleoplasmin were orthologs.

### Example 5

### Structure of the Npm2 Gene

The studies show that all three of the novel oocyte-specific cDNAs have open reading frames.

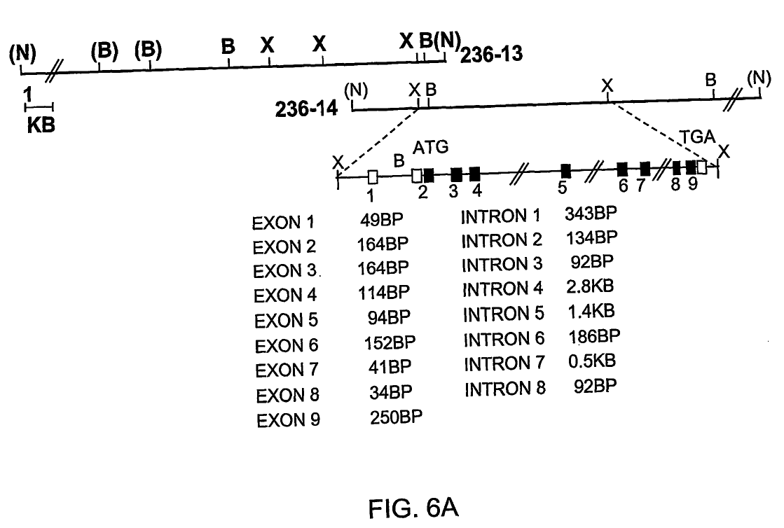

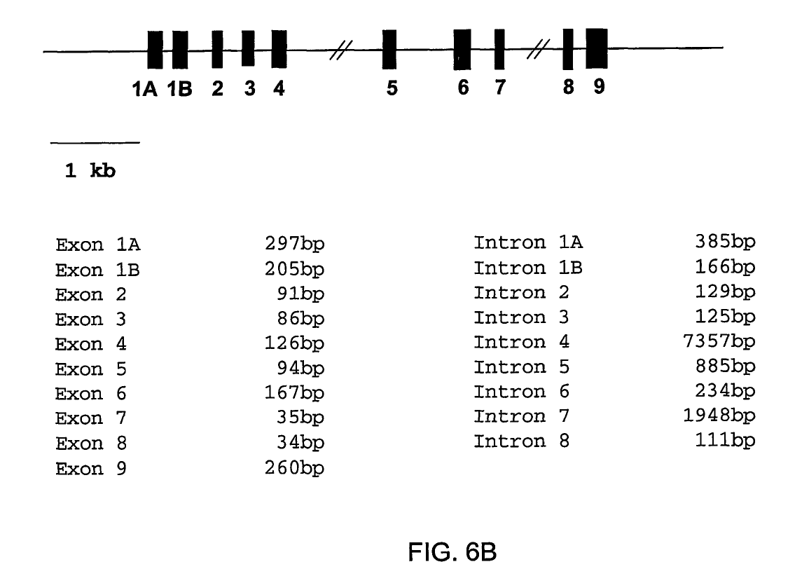

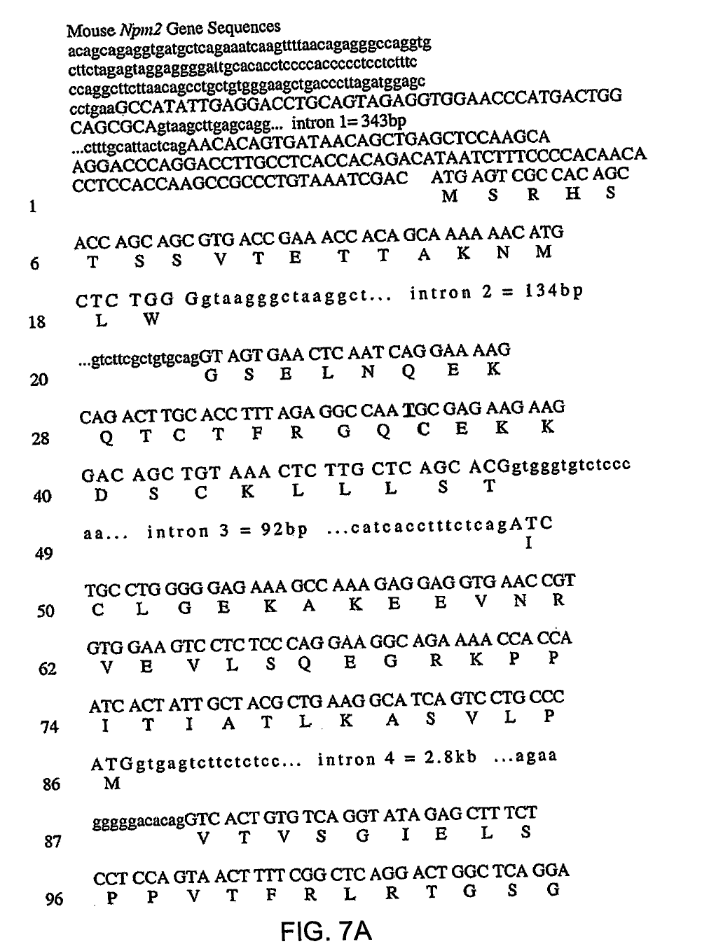

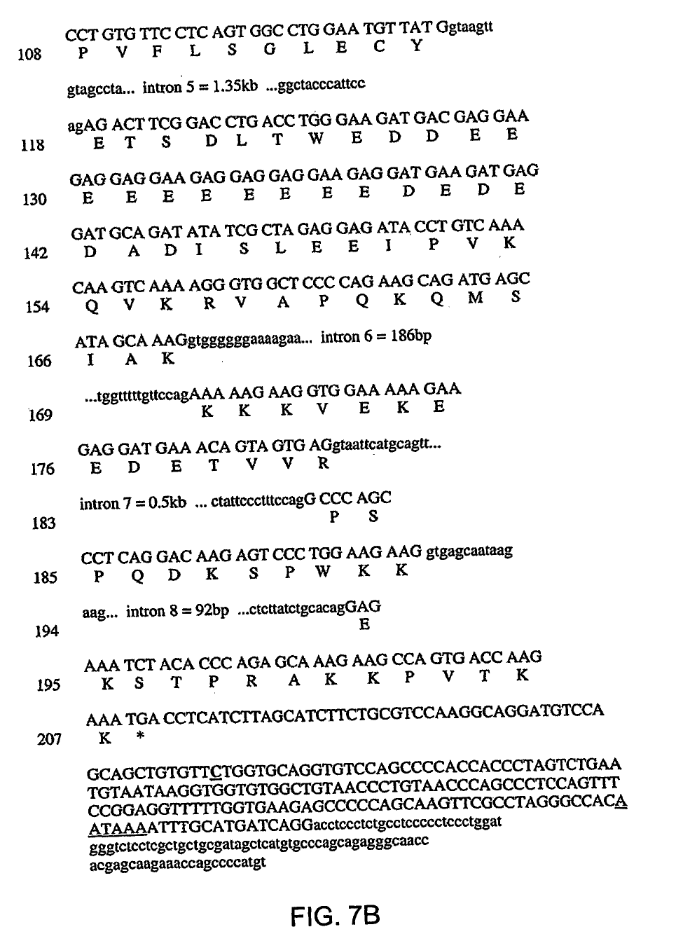

One of the fill length Npm2 cDNAs (clone 236-1) was used to screen a mouse 129/SvEv genomic library (Stratagene) to identify the mouse Npm2 gene. 500,000 phage were screened and 12 positive were identified. Two of these overlapping phage clones, 236-13 and 236-14 (˜37 kb of total genomic sequence), were used to determine the structure of the mouse Npm2 gene. The mouse Npm2 was encoded by 9 exons and spans 6.6 kb (FIGS. 6 and 7A and 7B (SEQ ID NO: 7A)). Two moderate size introns (introns 4 and 5) contributed the majority of the gene size. The initiation ATG codon resided in exon 2 and the termination codon in exon 9. The splice donor and acceptor sites (FIGS. 7A and 7B (SEQ ID NO: 7)) matched well with the consensus sequences found in rodents, and all of the intron-exon boundaries conformed to the “GT-AG” rule (Senapathy et al., 1990). A consensus polyadenylation signal sequence was found upstream of the polyA tracts which were present in the two isolated cDNAs (FIGS. 7A and 7B (SEQ ID NO: 7).

### Example 6

### Chromosomal Mapping of the Mouse Npm2 Gene

Chromosomal mapping of genes in the mouse identifies candidate genes associated with spontaneous or induced mouse mutations. To further aid in the functional analysis of the isolated novel ovary-specific cDNAs, these mouse genes were mapped using the Research Genetics Radiation Hybrid Panel. Table 3 shows the genes that have been mapped using this technique. Also, identification of the syntenic region on the human chromosome may identify one or more of these novel ovarian genes as candidate genes for known human diseases which map to these regions.

TABLE 3
Analysis of partial or full-length cDNAs

| pO1 cDNA | ORF | Database Homolog |
|---|---|---|
| O1-180 | 361 aa | No |
| O1-184 | 426 | No |
| O1-236 | 207 | Yes; Xenopus laevis |
| | | nucleoplosmin homolog |
| | | (81% similar) |

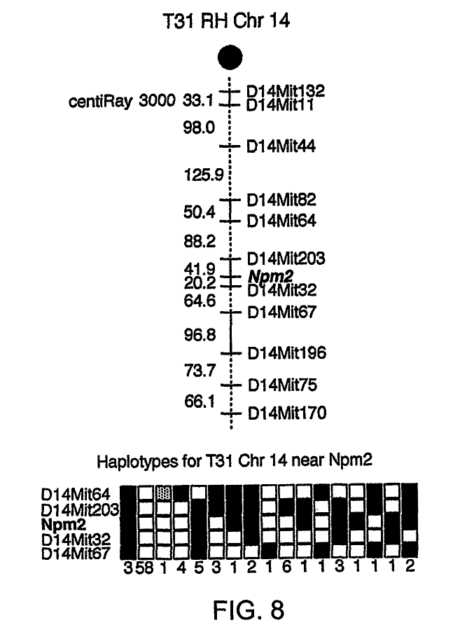

To map the mouse Npm2 gene, the inventors used the Research Genetics radiation hybrid panel, The Jackson Laboratory Backcross DNA Panel Mapping Resource, and The Jackson Laboratory Mouse Radiation Hybrid Database. Forward (SEQ.ID.NO.17: GCAAAGAAGCCAGTGACCAAGAAATGA) and reverse (SEQ.ID.NO.18: CCTGATCATG CAAATTTTATTGTGGCC) primers within the last exon were used to PCR amplify a 229 bp fragment from mouse but not hamster. Using these primers, the mouse Npm2 gene was mapped to the middle of chromosome 14 (FIG. 8). Npm2 showed linkage to D14Mit32 with a LOD of 11.2 and also had a LOD of 7.8 to D14Mit203. This region was syntenic with human chromosome 8p21.

### Example 7

### Generation of Knockout Mice Lacking Novel Ovary-Expressed Genes

Using the gene sequences obtained above, the inventors generate a targeting vector to mutate the O1-180, O1-184 and O1-236 genes in embryonic stem (ES) cells. These targeting vectors are electroporated into the hprt-negative AB2.1 ES cell line and selected in HAT and FIAU. Clones are processed for Southern blot analysis and screened using 5′ and 3′ external probes. ES cells with the correct mutation are injected into blastocysts to generate chimeras and eventually heterozygotes and homozygotes for the mutant O1-180, O1-184 and O1-236 genes.

Since expression of O1-180, O1-184 and O1-236 was limited to the ovary, the inventors anticipate that these O1-180-knockout, O1-184-knockout and O1-236-knockout mice are viable, but that females lacking these gene products can have fertility alterations (i.e., be infertile, subfertile, or superfertile). Mutant mice are analyzed for morphological, histological and biochemical information relating to intraovarian proteins required for folliculogenesis, oogenesis, or fertilization using techniques well within the ability of the person of ordinary skill in the art. It is envisioned that the absence of these proteins can result in female mice having increased or decreased fertility. These studies will lead a search for human reproductive conditions with similar idiopathic phenotypes.

### Example 8

### Generation of O1-184 Transgenic Animals

The O1-184 gene is flanked by genomic sequences and is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish. These animals are generated to overexpress O1-184 or express a mutant form of the polypeptide.

### Example 9

### Ovarian-Specific Expression of Mouse Npm2

To define the cell-specific expression of the Npm2 gene product, in situ hybridization analysis was performed using wild-type mouse ovaries.

In situ hybridization was performed as described previously (Albrecht et al., 1997; Elvin et al., 1999). Briefly, ovaries were dissected from C57B16/129SvEv mice and fixed overnight in 4% paraformaldehyde in PBS before processing, embedding in paraffin and sectioning at 5 um. The fragment O1-236 was used as the template for generating sense and antisense strands with [α³²P]-dUTP using the Riboprobe T7/SP6 combination system (Promega). Hybridization was carried out at 50-55° C. with 5×10⁶ cpm for each riboprobe per slide for 16 hours in 50% deionized formamide/0.3 M NaCl/20 mM Tris-HCl (pH 8.0)/5 mM EDTA/10 mM NaPO₄ (pH8.0)/10% dextran sulphate/1×Denhardts/0.5 mg/ml yeast RNA. High stringency washes were carried out in 2×SSC/50% formamide and 0.1×SSC at 65° C. Dehydrated sections were dipped in NTB-2 emulsion (Eastman Kodak, Rochester, N.Y.) and exposed for 4-7 days at 40° C. After the slides were developed and fixed, they were stained with hematoxylin and mounted for photography.

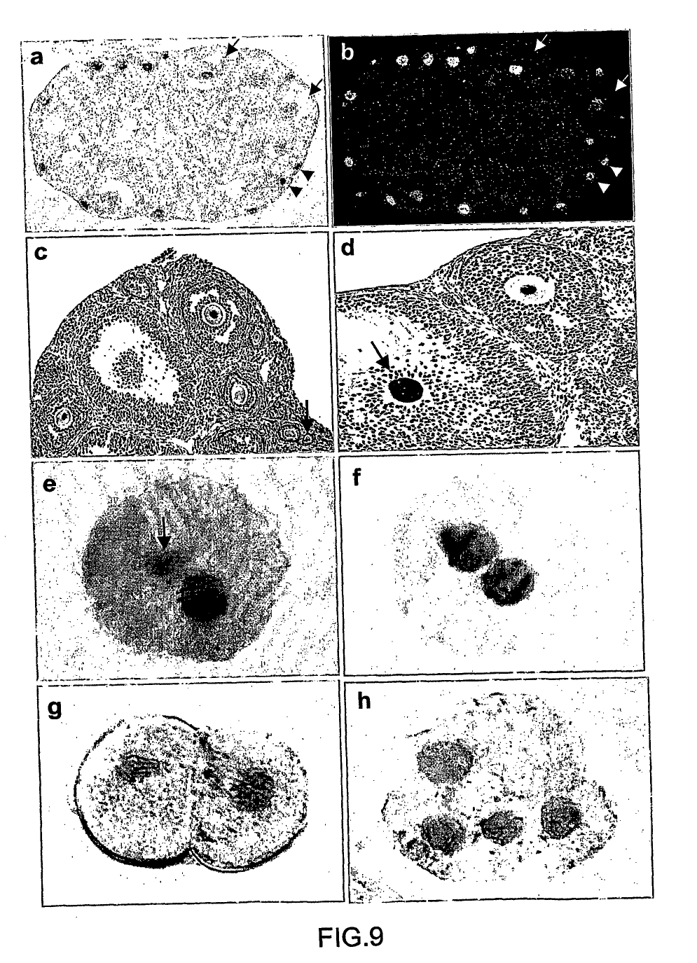

The Npm2 gene product was oocyte-specific (FIGS. 9A and 9B). The probe demonstrates specific expression in all growing oocytes. Oocyte-specific expression is first seen in the early one layer primary follicle (type 3a), with higher expression in the one layer type 3b follicle and all subsequent stages including antral (an) follicles. The “sense” probe does not detect a signal for this oocyte-specific gene.

### Example 10

### Subcellular Localization of NPM2

The subcellular localization of NPM2 protein was determined by immunohistostaining of mouse ovaries with anti-NPM2 antibodies.

The cDNA encoding the full-length mouse NPM2 protein was amplified by PCR to introduce a BamH1 site before the start codon and a XhoI site before the stop codon. This PCR fragment was cloned into pET-23b(+)(Novagen) to produce a His-tagged NPM2 protein and sequenced to confirm the absence of mutations. The recombinant NPM2 protein was purified as described in the pET System Manual (Novagen). Two goats were immunized with the purified His-tagged NPM2 to produce specific and high affinity antibodies.

Ovaries were fixed in 4% paraformaldehyde in PBS for 2 h, processed, embedded in paraffin, and sectioned at 5 um thickness. Goat anti-NPM2 polyclonal antiserum was diluted 1:2000 in Common Antibody Dilute (BioGenex). The pre-immune goat serum from the same goat was used as a control. All section were blocked for 10 min in Universal Blocking Reagent (BioGenex), and incubated with the primary antibody for 1 h at room temperature. NPM2 detection was accomplished using anti-goat biotinylated secondary antibody, streptavidin-conjugated alkaline phosphatase label and New Fuschin substrate (BioGenex Laboratories, Inc., San Ramon, Calif.).

One to eight-cell embryos and blastocysts were fixed in 4% paraformaldehyde in PBS for 2 h in 96-well round bottom plate, washed with 0.85% saline, and embedded in a few drops of 1.5% agarose. The agarose-containing embryos were dehydrated, embedded in paraffin, and analyzed as described above.

Consistent with the expression pattern of Npm2 mRNA, NPM2 protein was expressed in oocytes from type 3 to antral follicle stages. In randomly cycling mice, the anti-NPM2 antibody strongly and specifically stained the nucleus (FIG. 9C). The oocyte nucleus is also called the germinal vesicle (GV). The preovulatory surge of luteinizing hormone (LH) accelerates the maturation of GV oocytes and promotes GV breakdown (GVB). When mice were injected with PMSG and hCG to induce superovulation, the NPM2 protein redistributes in the oocytes of antral follicles after germinal vesicle breakdown. In preovulatory GVB oocytes, the NPM2 was evenly distributed in the cytoplasm of the oocyte (FIG. 9D). Since xNPM2 has been implied to play a role in sperm DNA decondensation and pronuclei formation after fertilization, this redistribution suggested that the cytoplasmic NPM2 was now properly positioned to interact with the sperm nucleus at the time of fertilization. To examine the NPM2 expression after fertilization, early embryos were fixed, sectioned and stained with anti-NPM2 antibodies. In zygotes, NPM2 began to translocate back to the nucleus. FIG. 9E shows an intermediate stage in which one pronucleus was formed but other was not yet complete and some NPM2 was still present in the cytoplasm. At a later point (FIG. 9F), all of the NPM2 was present in the pronuclei. In two-cell (FIG. 9G) and eight-cell (FIG. 9H) embryos, the antibody continued to detect the NPM2 protein exclusively in the nucleus. NPM2 continued to be detected at significantly reduced levels in blastocysts (embryonic day 3.5), but in embryonic day 6.5 embryos, NPM2 expression was undetectable.

### Example 11

### Targeted Disruption of the Mouse Npm2 Gene and Generation of Npm2 Knockout Mice

To study the role of NPM2 in mammalian oocyte development and early embryo development, the inventors disrupted the mouse Npm2 locus using ES cell technology.

A targeting vector for Npm2 was constructed to delete exons 2 and 3 and the splice junction of exon 4. The deletion targeting vector contains from left to right, 2.2 kb of 5′ Npm2 homology, a PGK-hprt expression cassette, 4.6 kb of 3′ Npm2 homology and an MC1-tk (thymidine kinase) expression cassette. The linearized Npm2 targeting vector was electroporated into AB2.1 ES cells. ES cell clones were selected in M15 medium containing HAT hypoxanthine, aminopterine and thymidine and FIAU [1-(2′-deoxy-2′-fluoro-B-D-arabinofuranosyl)-5′-iodouracil]. Culturing of ES cells and collection and injection of blastocysts have been previously described by Matzuk et al., 1992. For genomic Southern blot analysis, BglII-digested DNA was transferred to GeneScreen Plus nylon membrane and probed with an external 190 bp PCR synthesized fragment corresponding to exon 9 sequence (3′ probe). An internal 200 bp PCR synthesized fragment (49 bp exon 1 plus 150 bp 5′ upstream sequence) was also used to distinguish the wild-type and Npm2 null (Npm2tm1Zuk) alleles when DNA was digested with BamHl. A PCR-synthesized probe containing the 137 bp exon 2 sequence was used to verify that exon 2 was absent in mice homozygous for the Npm2tm1Zuk allele when DNA was digested with Pst1. A single correctly targeted ES cell clone (named Npm2-118-B11) was expanded, and ES cells were injected into C57B1/6 blastocysts to obtain chimeric mice which ultimately produced C57B16/129/SvEv hybrid and 129/SvEv inbred F1 progeny.

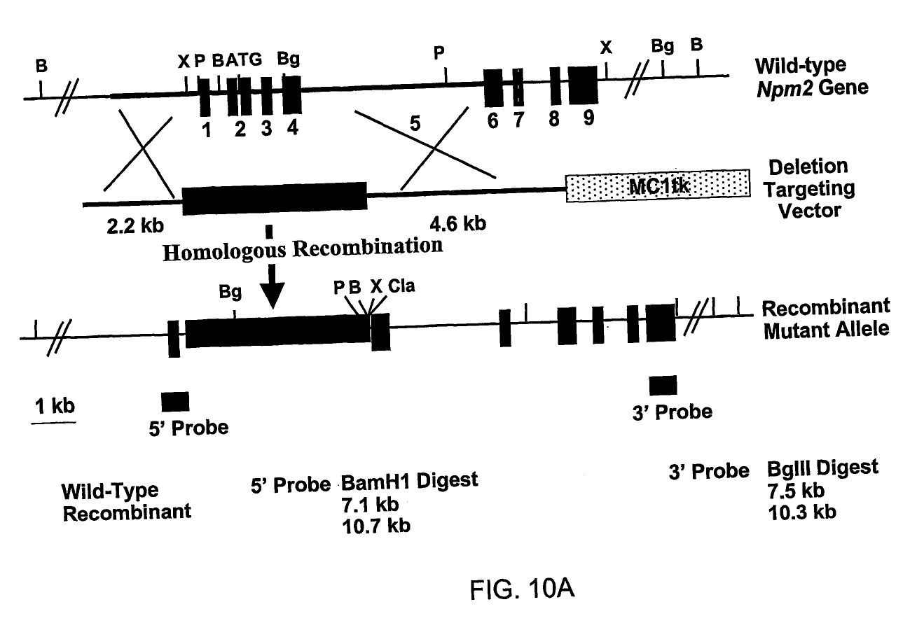

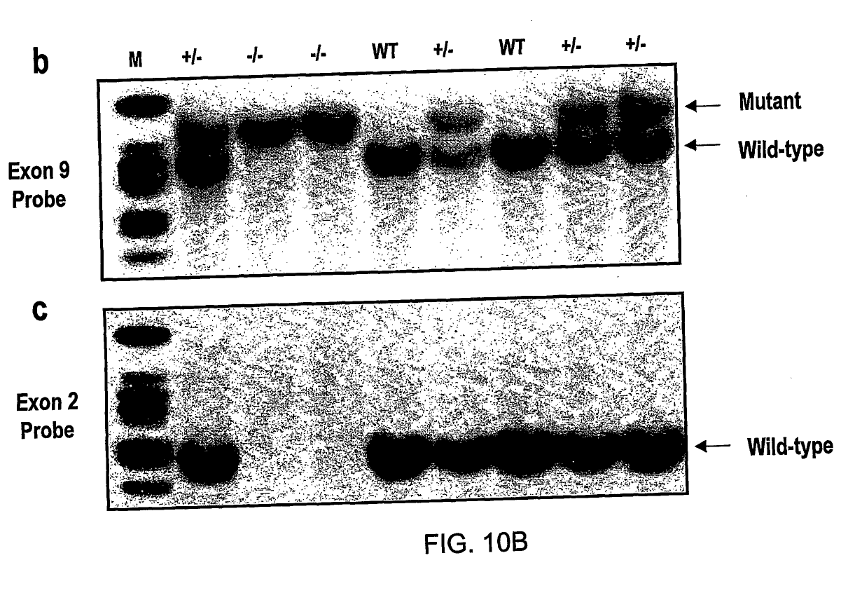

The targeting vector was constructed to delete exon 2 which contains the translation initiation codon and also exon 3 and the exon 4 splice junction (FIG. 10A). Outside of exon 2, only one other ATG was present in the remaining sequence (exon 6), and this ATG was positioned downstream of the acidic domain and between the bipartite nuclear localization consensus sequence. Thus, this vector generated an Npm2 null allele. F1 heterozygous (Npm2tm1Zuk/+; herein called Npm2^{+/-}) mice were viable and fertile, and were intercrossed to investigate the developmental consequences of NPM2 absence. Genotype analysis of 230 F2 offspring from these intercrosses (FIG. 10B; Table 4) was consistent with a normal Mendelian ratio of 1:2:1, and a similar number of male and female homozygotes (Npm2^{-/-}) were produced. Therefore, Npm2 homozygous mutant male and female mice were viable and appeared to have normal sexual differentiation demonstrating that Npm2 was not required prior to birth.

TABLE 4
Heterozygous mating

| | −/− | +/− | Wild type | Total |
|---|---|---|---|---|
| Male | 27 | 71 | 19 | 117 |
| Female | 27 | 53 | 33 | 113 |
| Total | 54 | 124 | 52 | 230 |
| % | 23 | 54 | 23 | 100 |

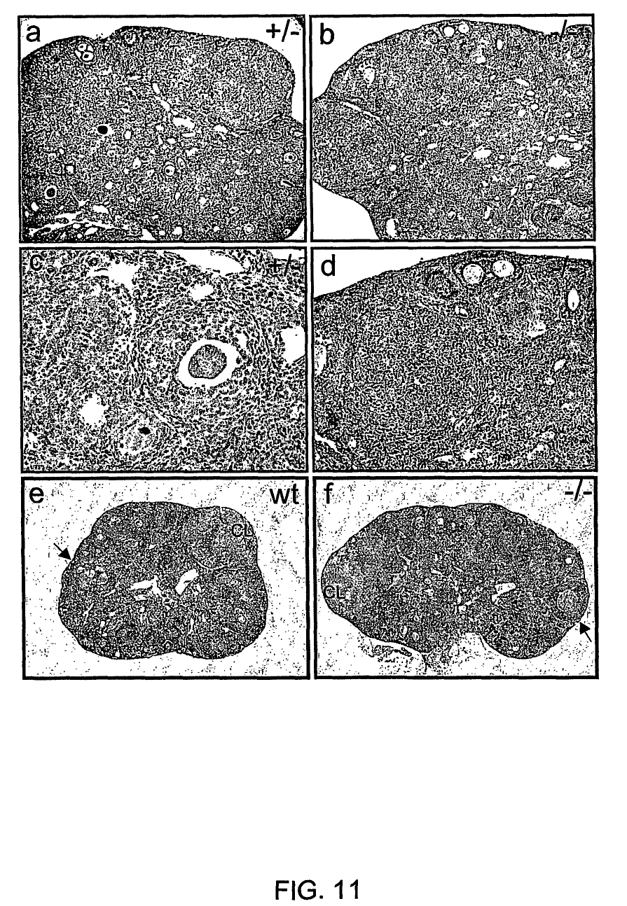

To confirm that the mice genotyped as Npm2 homozygotes lacked Npm2, a cDNA probe that hybridized to exon 2 of the wild-type Npm2 gene was used for Southern blot analysis. As shown (FIG. 10C), this probe failed to detect any signal in DNA derived from homozygous (Npm2^{-/-}) mice in which exon 2 had been deleted. Furthermore, Npm2 immunohistochemical analysis was performed on Npm2 homozygotes and controls. Whereas the expression of NPM2 protein was noted in the ovaries from the heterozygous controls (FIG. 11A and 11C), no protein was detected in oocytes in the homozygote ovaries (FIG. 11B and 11D). This confirmed that the Npm2tm1Zuk mutation was a null allele and that Npm2 homozygotes were completely lacking NPM2 protein.

### Example 12

### Loss of Npm2 Results in Female Infertility and Subfertility

To study the function of NPM2 in reproductive function, adult homozygous hybrid (C57B1/6/129SvEv) male or female mice were intercrossed with control hybrid mice (C57B1/6/129SvEv) mice. Consistent with the female-specific expression of Npm2 mRNA and protein, Npm2^{-/-} male mice were fertile and had no gross or histological defects in the testes (data not shown). Similarly, intercrosses of 10 female Npm2 heterozygotes with heterozygous males during a 5-8 month period resulted in 53 litters with 8.55 offspring/litter (0.97 litters/month)(Table 5). In contrast, only 9 out of 12 Npm2^{-/-} female mice became pregnant over a 5-8 month period resulting in 32 litters with an average of 2.75 offspring/litter (0.43 litters/month)(Table 5). Thus, deficiency of Npm2 leads to subfertility and infertility in females but not males.

TABLE 5
Matings of Npm2 Knockout Mice

| | | | | | Average | |
|---|---|---|---|---|---|---|
| Genotype of Parents | | | | | Litter size | Litters/month |
| Male | | Female | Mothers | Litters | (Mean ± SEM) | (Mean ± SEM) |
| +/− | X | +/− | 8 | 51 | 8.55 ± 0.34* | 0.97 ± 0.03** |
| WT | X | −/− | 12*** | 32 | 2.75 ± 0.25* | 0.43 ± 0.10** |

*P < 0.001
**P < 0.005
***Three mice are infertile

### Example 13

### Early Cleavage Defect in Npm2-Null Fertilized Eggs

To determine the causes of the fertility defects in the Npm2^{-/-} female mice, ovaries were first examined morphologically and histologically. There is no significant difference between Npm2^{-/-} and control ovaries at the gross or histological levels (FIGS. 11E and 11F). Normal folliculogenesis including the formation of corpora lutea were observed in the Npm2^{-/-} ovaries suggesting that ovulation occurred in these mice.

To confirm that ovulation was occurring and to further study the cause of the infertility and subfertility of the Npm2^{-/-} mice, pharmacological superovulation of wild-type, heterozygous, and homozygous mice was performed and the eggs were collected from the oviducts and cultured in vitro.

Immature (21-24 day old) females were injected intraperitoneally with 5 IU PMSG (pregnant mare serum gonadotropin) followed by injection of 5 IU hCG (human chorionic gonadotropin) to induce superovulation as described (Hogan et al., 1994; Matzuk et al., 1996). The injected mice were mated to wild-type male mice. Eggs were harvested the next morning from the oviducts of these mice. Cumulus cells were removed from the eggs by using 0.3 mg/ml hyaluronidase in M2 medium (Sigma). Eggs were cultured in M16 Medium (Sigma) covered with light paraffin oil in a humidified 37° C. incubator with an atmosphere of 5% CO₂ and 95% air (Hogan et al., 1994).

For the staining of DNA, eggs were washed once in PBS, incubated in 4% paraformaldehyde in PBS containing 10 ug/ml bisbenzimide (Hoechst 33258) for 20 min at room temperature, washed twice with PBS, mounted with Fluoromount-G, and photographed by using fluorescence microscopy (Axioplan 2 imaging, Carl Zeiss).

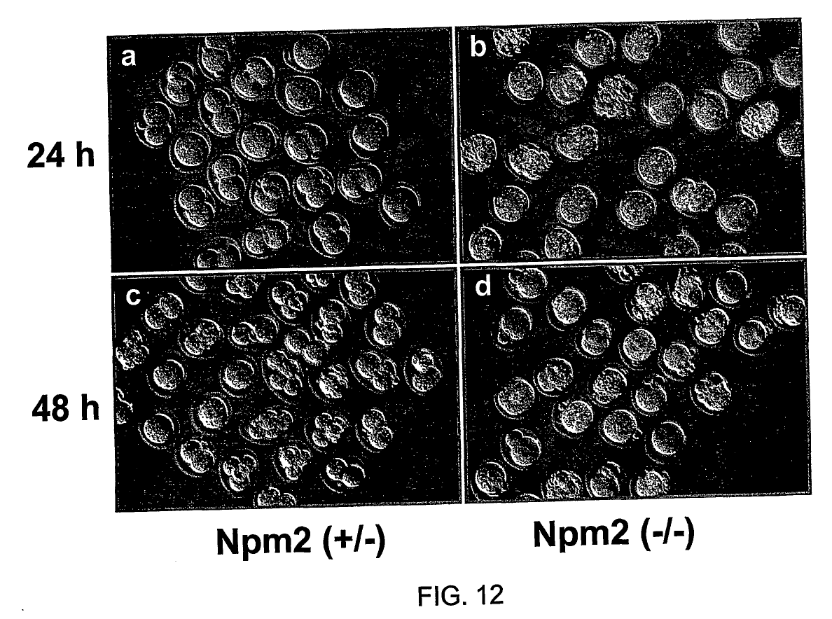

Pregnant mare serum gonadotropin/human chorionic gonadotropin superovulation treatment of 21-24 day old mice resulted in similar numbers of eggs ovulated in Npm2^{-/-} females compared to wild-type or heterozygote controls (Table 6). The eggs from Npm2^{-/-} mice appear to be fertilized by spermatozoa normally because there were no significant differences between the Npm2^{-/-} and controls in the formation of the second polar body, evidence of fertilization. However, there was a substantial defect in the cleavage of one cell embryos into two cell embryos in the Npm₂^{-/-} mice (Table 6, FIG. 12A-12D). Besides a significant reduction in the number of two cell embryos, some bizarre, developmentally-abnormal embryos appeared during the 24 hours of in vitro culture (FIG. 12B). Unlike control eggs (FIG. 12C), Npm2^{-/-} eggs could not progress to the four cell embryo stage the current in vitro culture assay (FIG. 12D). Thus, the defect in the Npm2^{-/-} mice appeared to result in a reduced viability of embryos.

TABLE 6
In vitro culture of eggs released by superovulation

| | Number | | Presence of | 2 cell embryo |
|---|---|---|---|---|
| | of | Eggs | polar body | (Mean ± SEM |
| Genotype | females | (Mean ± SEM) | (Mean ± SEM) | (%) |
| Wild type | 7 | 14.4 ± 3.8 | 8.6 ± 1.4 | 7.3 ± 2.1** |
| | | | | (50.5%) |
| Npm2^{±} | 21 | 12.6 ± 2.1 | 6.9 ± 0.9 | 7.1 ± 1.3** |
| | | | | (56.3%) |
| Npm2^{-/-} | 15 | 15.7 ± 3.9 | 7.2 ± 1.7 | 1.3 ± 0.4** |
| | | | | (8.3%) |

*Percentage of 2 cell embryos developed from total eggs
**P < 0.001

### Example 14

### Structure of the O1-180 Gene and O1-180 Pseudogene

A ZAP-express mouse ovary cDNA library was screened to isolate the full-length O1-180 cDNA. Excluding the polyA tail, the ful-length O1-180 cDNA is about 1.3 kb, and encodes an open reading frame from nucleotides 26 to 1108. The O1-180 cDNA is homologous to several ESTs in the database, including ESTs in a mouse sixteen-cell embryo CDNA library (AU044294) and a mouse unfertilized egg cDNA library (AU023153). The polypeptide predicted from the O1-180 cDNA ORF consists of 361 amino acids, with a molecular mass of 40 IDa. Searching the public protein database failed to identify any known protein homologues. A bipartite nuclear localization signal was found at positions 333 to 350 (SEQ.ID.NO.19: Lys-Arg-Pro-His-Arg-Gln-Asp-Leu-Cys-Gly-Arg-Cys-Lys-Asp-Lys-Arg-Leu-Ser), strongly suggesting that O1-180 may migrate to the oocyte or embryo nucleus.

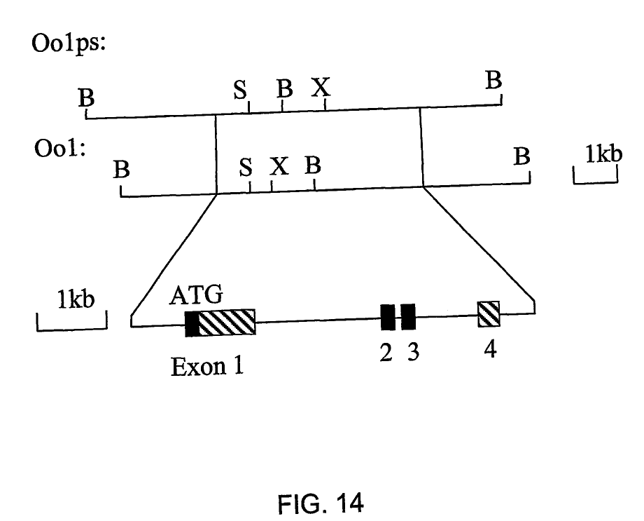

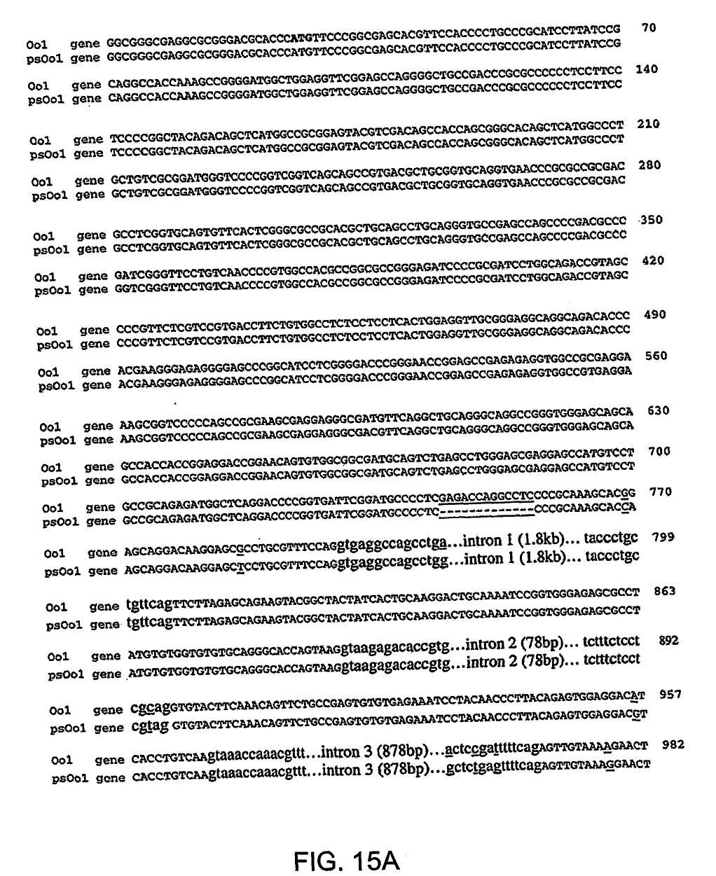

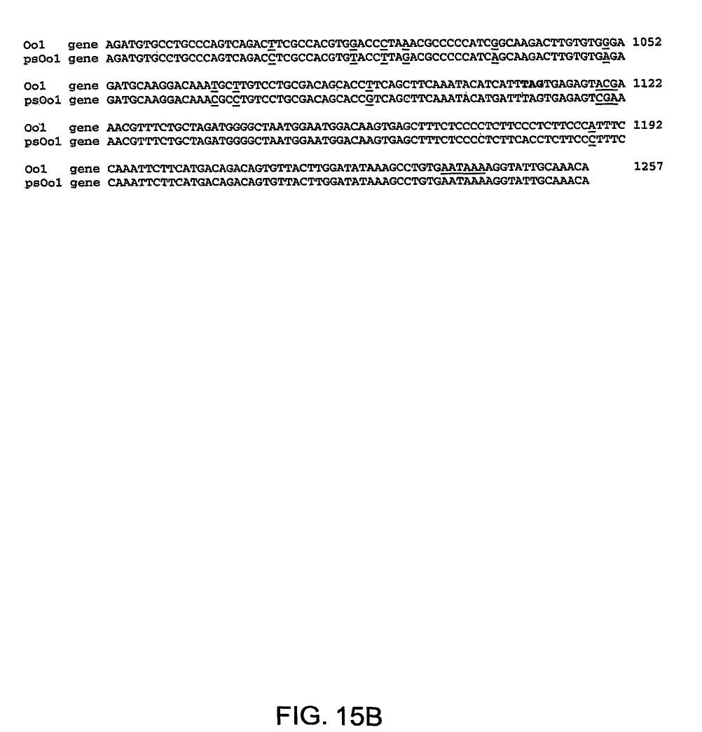

To clone the mouse O1-180 gene, a mouse genomic λ Fix II phage library generated from mouse 129SvEv strain was screened with the full length O1-180 cDNA. Twelve independent λ recombinant clones were isolated; eight of which were identified as unique clones and were further characterized by subcloning, Southern blot analysis, and sequencing. Surprisingly, only one genomic insert DNA starting 650 nucleotide upstream of exon 2 of the gene corresponded to the 3′-portion of the O1-180 gene. The remaining clones corresponded to a closely related gene, in which the exons share 98% identity with O1-180 eDNA. Based on the exon differences, O1-180 gene- and the related gene-specific primers were designed and reverse transcription-polymerase chain reactions (RT-PCR) were performed. cDNAs from 8-week-old C57 mouse tissues, including brain, heart, lung, spleen, liver, small intestine, stomach, kidney, uterus, testis, and ovary, were used as templates. Consistent with the Northern blot analysis, O1-180 cDNA was amplified exclusively in the ovary; while the related gene cDNA was not detectable in any of the tissues. This indicated that the related gene isolated from the mouse genomic λ Fix II phage library was a pseudogene. A BAC 129SvJ mouse genomic library was screened by PCR with two sets of O1-180 gene-specific primers, and only one BAC clone was isolated. Sequencing of the entire coding region and exon-intron boundaries of the BAC and λ phage clones showed that both the O1-180 and the O1-180 pseudogene contained four exons and three introns (FIG. 14). As shown in FIG. 15, all of the exon-intron boundaries satisfied the GT-AG intron donor-acceptor splice rule. The major difference between the O1-180 gene and the pseudogene was a 13-nt gap in exon 1 of the pseudogene, which the inventors expect results in a frame shift and early termination in exon 2 of the pseudogene. The sequences of exon 2 in both the O1-180 gene and pseudogene were identical, and there are single base pair mutations in exons 3 and 4 (FIG. 15) (SEQ ID NO:11 and SEQ ID NO:12).

### Example 15

### Mouse Chromosome 5

The whole genome-radiation hybrid panel T31 McCarthy et al., 1997) were purchased from Research Genetics (Huntsville, Ala.) and used according to the manufacturer's instruction. The panel was constructed by fusing irradiated mouse embryo primary cells (129aa) with hamster cells. Because the sequence of the hamster homologues for O1-180 is unknown, the inventors designed the reverse primers from the 3′-untranslated region of the murine sequence to mnimize the risk of coamplification of the hamster homologues (Makalowsld and Boguski, 1998). O1-180 gene specific primers were (SEQ.ID.NO.20) 5′-CTAGAAAAGGGGACTGTAGTCACT-3′ forward, and (SEQ.ID.NO.21) 5′-TGCATCTCCCACACAAGTCTTGCC-3′ reverse; pseudo O1-180 gene specific primers were (SEQ.ID.NO.22) 5′-CTAGAAAAGGGGACTATAGGCACC-3′ forward, and (SEQ.ID.NO.23) 5′-TGCATCTCTCACACAAGTGTTGCT-3′ reverse. Specificity of the two sets of primers was tested with A23 hamster DNA and 129 mouse DNA. The PCR reactions were performed in 15 μl final volume, containing 1 μl of each panel DNA, 1.25 u of Taq platinum DNA polymerase (Gibco, Rockville, Md.), companion reagents (0.25 mM dNTPs, 1.5 nM MgCl2, 1×PCR buffer), and 0.4 μM of each primer. An initial denaturation step of 4 min at 94° C. was followed by amplification for 30 cycles (40 s at 94° C., 30 s at 60° C., and 30 s at 72° C.) and final elongation at 72° C. for 7 min.

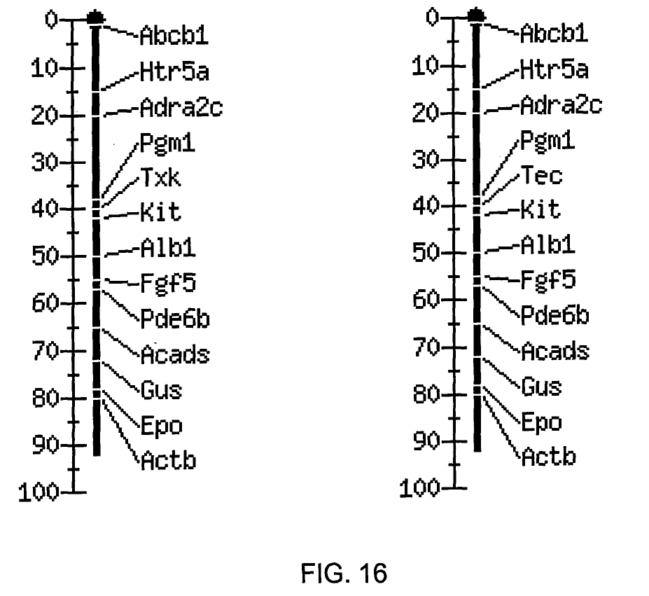

Both O1-180 gene and O1-180 pseudogene specific primers were designed respectively, and all 100 of the cell line DNAs of the T31 Mouse Radiation Hybrid Panel were screened by PCR in a duplicate assay. The data for each gene were submitted for analysis at the Jackson Laboratory Mouse Radiation Hybrid Mapper Server. Both genes were placed in the same region on mouse Chromosome 5. The O1-180 locus is at 40cM, between two markers D5Buc48 and Txk, while the O1-180 pseudogene lies at 41cM, between Tec and D5Mit356, just distal to the coding locus (FIG. 16). This is syntenic to a region in humans Chromosome 4p12.

### Example 16

### Localization of O1-180 in Mouse Ovaries

In situ hybridization was performed with the O1-180 specific probe. [α-³⁵S]UTP-labeled antisense and sense probes were generated by the Riboprobe T7/T3 combination system (Promega, Madison, Wis.). Hybridization was carried out according to methods described by Albrecht et al., 1997 and Elvin et al., 1999A.

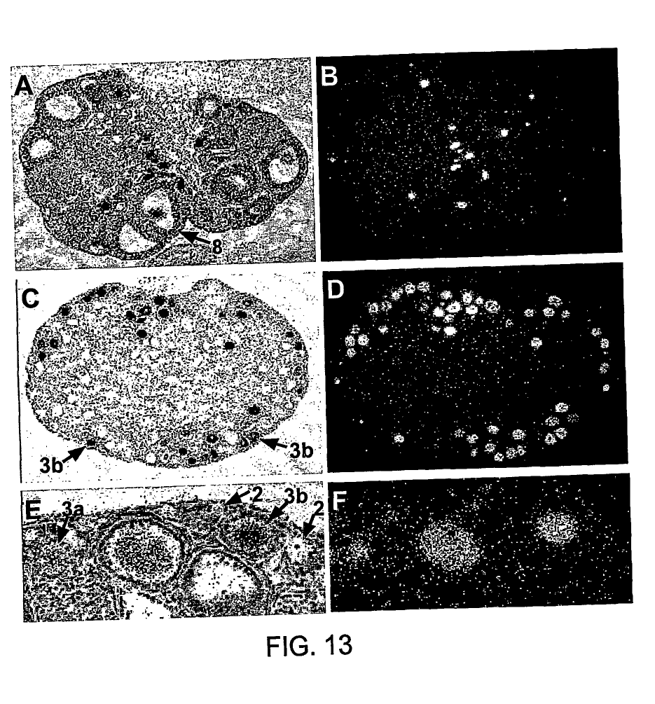

In situ hybridization showed high level expression of O1-180 localized to the oocytes within these ovaries. The expression of O1-180 within oocytes was evident at the one-layer (primary) follicle stage through the antral follicle stage, but no expression was observed at the primordial follicle stage. Because the number of follicles is increased in Gdf9 knockout ovaries due to the arrest of follicle development at the primary follicle stage, more O1-180 positive oocytes were detected in each section (FIG. 13).

### Example 17

### Analysis of O1-180

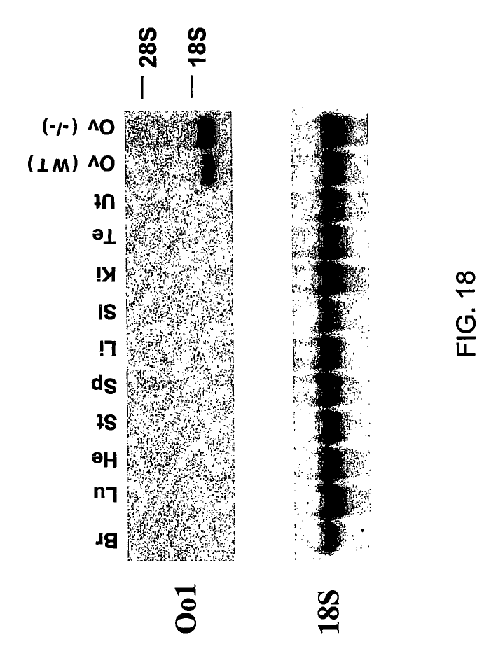

Northern blot analysis was performed using standard techniques well known and used in the art. Briefly, ovarian mRNA was isolated from wildtype and GDF-9(−/−) mice. FIG. 18 shows that O1-180 is specific for ovarian tissue.

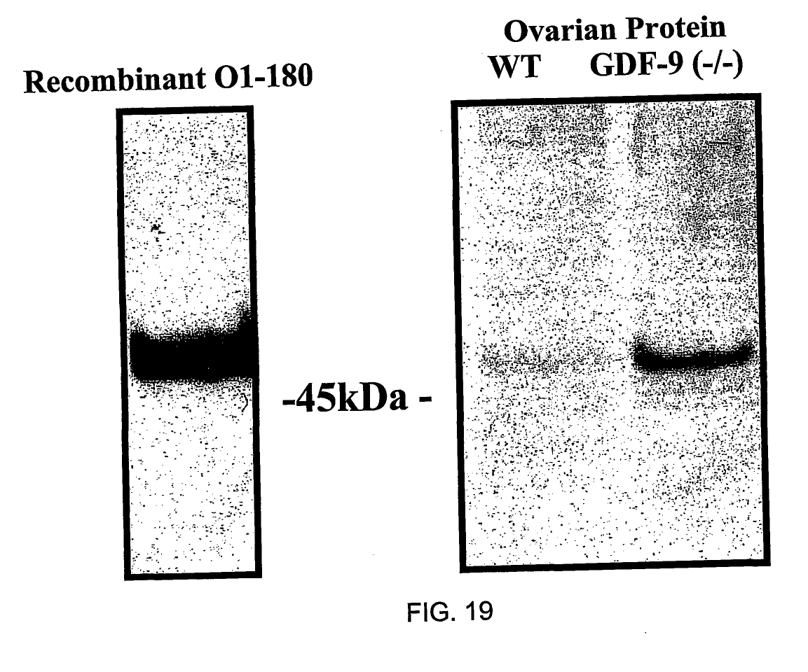

Western blot analysis was performed using standard techniques well known and used in the art. Briefly, ovarian protein was isolated from wildtype and GDF-9(−/−) mice. Antibodies to O1-180 were used to compare the size of the recombinant O1-180 protein to a native O1-180 protein. FIG. 19 revealed that the recombinant O1-180 protein is similar in size to the native O1-180 protein from isolated ovaries from GDF-9(−/−) mice.

### Example 18

### Subcellular Localization of O1-180

The subcellular localization of O1-180 protein was determined by immunohistostaining of mouse ovaries with anti-O1-180 antibodies.

The cDNA encoding the full-length mouse O1-180 protein was amplified by PCR to introduce a BamH1 site before the start codon and a XhoI site before the stop codon. This PCR fragment was cloned into pET-23b(+)(Novagen) to produce a His-tagged O1-180 protein and sequenced to confirm the absence of mutations. The recombinant O1-180 protein was purified as described in the pET System Manual (Novagen). Two goats were immunized with the purified His-tagged O1-180 to produce specific and high affinity antibodies.

Ovaries were fixed in 4% paraformaldehyde in PBS for 2 h, processed, embedded in paraffin, and sectioned at 5 um thickness. Goat anti-O1-180 polyclonal antiserum was diluted 1:2000 in Common Antibody Dilute (BioGenex). The pre-immune goat serum from the same goat was used as a control. All section were blocked for 10 min in Universal Blocking Reagent (BioGenex), and incubated with the primary antibody for 1 h at room temperature. Npm2 detection was accomplished using anti-goat biotinylated secondary antibody, streptavidin-conjugated alkaline phosphatase label and New Fuschin substrate (BioGenex Laboratories, Inc., San Ramon, Calif.).

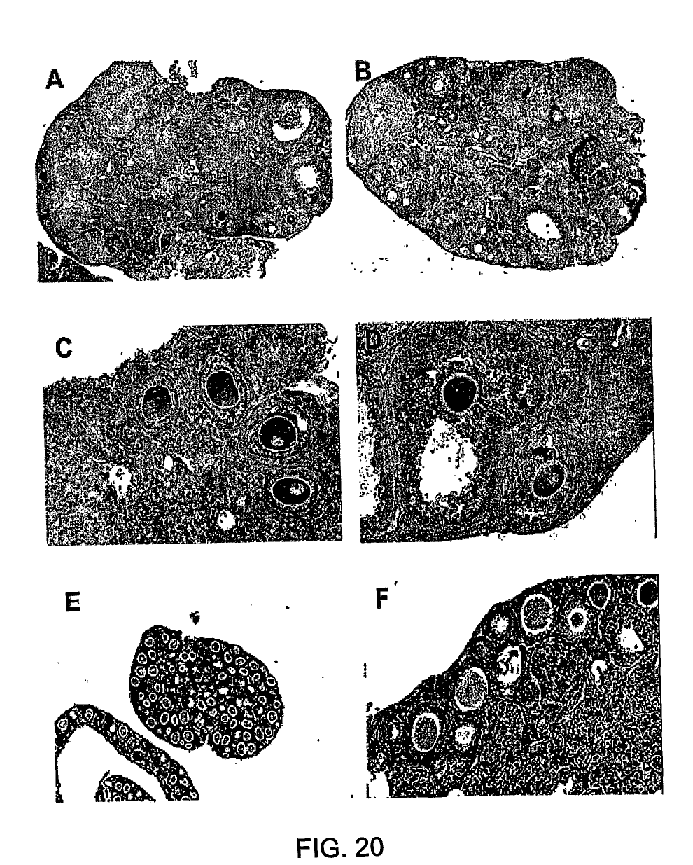

FIG. 20 shows immunostaning of O1-180 in mouse ovaries. As shown in FIG. 20, the O1-180 protein was localized specifically to the cytoplasm of mouse oocytes and zygotes but disappeared after this point.

### Example 19

### Generation of O1-180 Knockout Mice

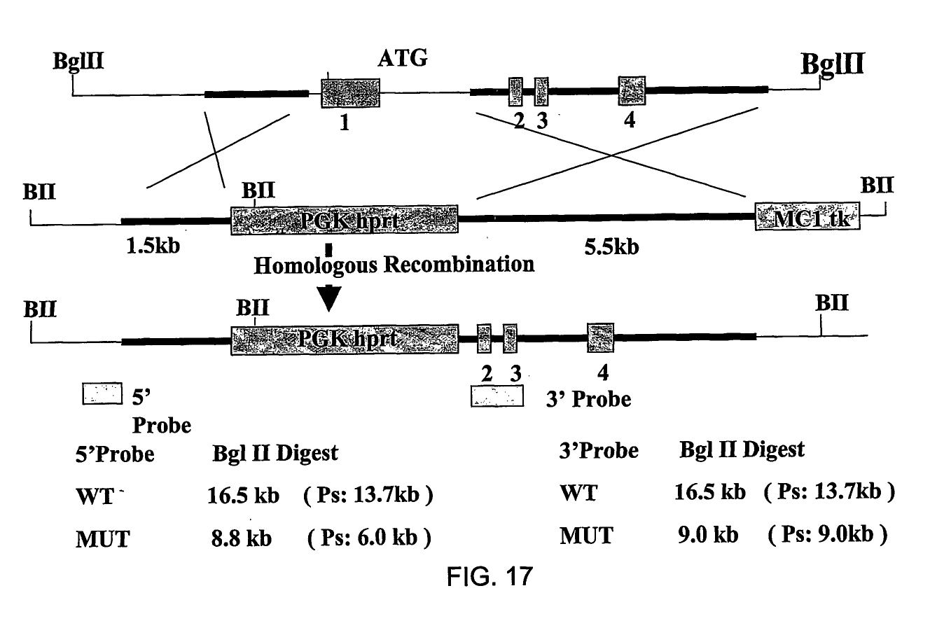

A targeting vector to mutate the O1-180 gene was constructed from the isolated sequences (FIG. 17). To study the role of O1-180 in mammalian oocyte development and early embryo development, the inventors disrupted the mouse O1-180 locus using ES cell technology. The targeting vector was constructed to delete exon 1 which contains the translation initiation codon. Thus, this vector generated an O1-180 null allele.

### Example 20

### Loss of O1-180 Results in Female Infertility and Subfertility

To study the function of O1-180 in reproductive function, adult homozygous hybrid (C57B1/6/129/vEv) male or female mice were intercrossed with control hybrid mice (C57B/1/6/129/SvEv) mice. Consistent with the female-specific expression of O1-180 mRNA and protein, O1-180^{-/-} male mice are fertile and had no gross or histological defects in the testes. Similarly, intercrosses of female O1-180 heterozygotes with heterozygous males during a 5-8 month period resulted in 7.1 offspring/litter (Table 7). In contrast, none of the O1-180^{-/-} female mice became pregnant over a 5-8 month period (Table 7). Thus, deficiency of O1-180 leads to subfertility and infertility in females but not males.

TABLE 7
Fertility of O1-180 Mice

| | | Average | | | | |
|---|---|---|---|---|---|---|
| | | Litter | Sex Ratio | | | |
| Breeder | | Size | (Female/ | Genotype of Pups | | |
| F | M | (pups/litter) | Male) | Wt | +/− | −/− |
| +/− | +/− | 7.1 | 1 | 53 (23%) | 121 (52%) | 59 (25%) |
| | | | (119/114) | | | |
| +/− | −/− | 8.8 | 0.94 | 0 | 111 (52%) | 101 (48%) |
| | | | (103/109) | | | |
| −/− | +/− | 0 | | | | |
| | or Wt | | | | | |

### Example 21

### Defect in O1-180-Null Fertilized Eggs

To determine the causes of the fertility defects in the O1-180^{-/-}, O1-180^{+/-} female mice, ovaries were first examined morphologically and histologically.

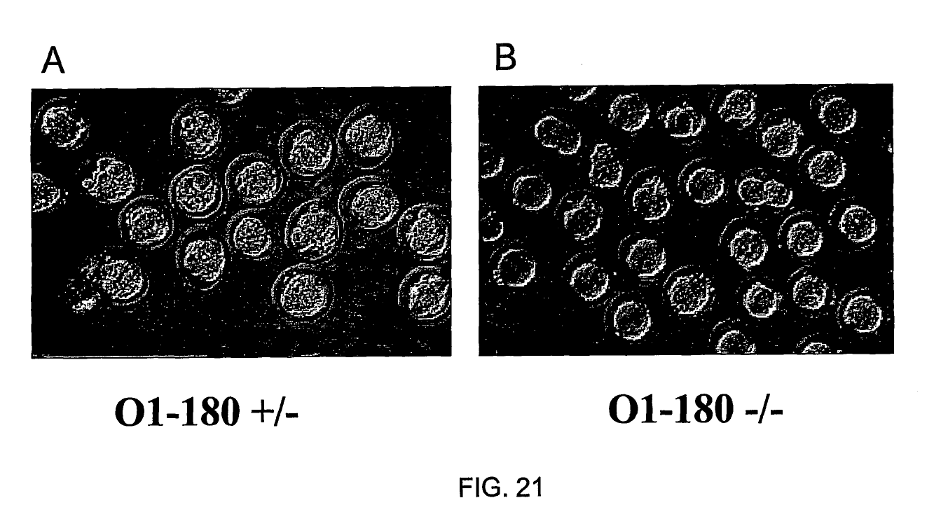

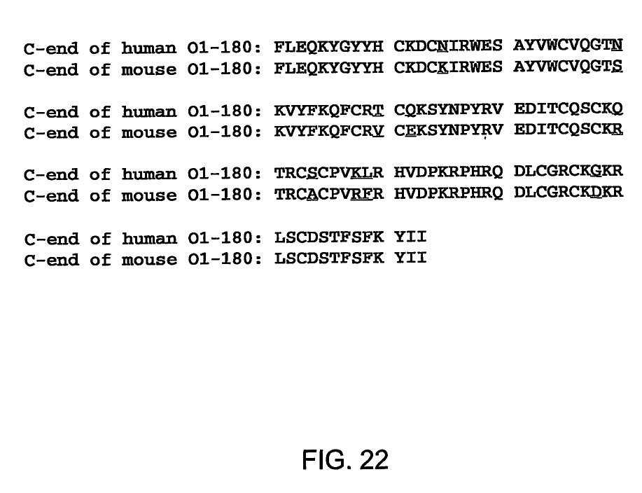

To confirm that ovulation was occurring and to further study the cause of the infertility and subfertility of the O1-180^{-/-}, O1-180^{+/-}, mice, pharmacological superovulation of wild-type, heterozygous, and homozygous mice was performed and the eggs were collected from the oviducts and cultured in vitro. As shown in FIG. 21 and Table 8, by day 2, only a few O1-180^{-/-} zygotes developed to 2-cell stage and none of them developed to a 4-cell stage by day 3. This was in contrast to the O1-180^{+/-} zygotes which most of them developed to a 2-cell embroyo by day 2 and of these most of them developed into blastocysts by day 3 and day 4.

Thus, the defect in the O1-180^{-/-} mice appeared to initiate after fertilization and before the development of the blastocyst.

TABLE 8
Embryonic Development of O1-180 Mutant Mice in vivo

| | O1-180 +/− (n = 8) | | | O1-180 −/− (n = 13) | | |
|---|---|---|---|---|---|---|
| | Total | | | Total | | |
| | Zygotes | 2-4-cell | >4-cell | Zygotes | 2-4-cell | >4-cell |
| | (D1) | (D2) | (D3) | (D1) | (D2) | (D3 |
| Mean | 31.63 | 28.25 | 23.63 | 34.31 | 7.15 | 0.00 |
| SD % | 13.51 | 11.70 | 13.60 | 14.84 | 5.44 | 0.00 |
| SEM | | 83.30 | 74.70 | | 20.83 | 0.00 |
| | 4.78 | 4.14 | 4.81 | 4.12 | 1.51 | 0 |

### Example 22

### Two Hybrid Analysis

Two hybrid screen is used to elucidate or characterize the function of a protein by identifying other proteins with which it interacts.

The protein O1-180 or O1-236 or O1-184 is made into a chimeric protein, which contains a DNA binding domain of GAL4 along with the DNA for the protein of interest. Plasmids containing nucleotide sequences which express this chimeric protein are transformed into eukaryotic cells, which also contain a representative plasmid from a library containing the GAL4 activation domain fused to different nucleotide sequences encoding different potential target proteins. If the protein of interest (O1-180, O1-236 or O1-184) physically interacts with a target protein, the GALA activation domain and GAL4 DNA binding domain are tethered and are thereby able to act conjunctively to promote transcription of a reporter gene. If no interaction occurs between the O1-180, O1-236 or O1-184 protein and the potential target protein in a particular cell, the GAL4 components remain separate and unable to promote reporter gene transcription on their own.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the ′ disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

### References

The following references, to the extent that they provide exemplary procedural or other details supplementary to those set forth herein, are specifically incorporated herein by reference:

Albrecht, U., et al., (1997). In Molecular and Cellular Methods in Developmental Toxicology, G. P. Daston, ed. (Boca Raton, Fla., CRC Press), pp. 23-48.

Burglin, T., et al., (1987). Genes Dev 1, 97-107.

Carabatsos M., et al., (1998). Dev. Biol. 203, 373-384.

Chan, W. Y., et al., (1989). Biochemistry 28, 1033-9.

Channing, C. P., (1970). Recent Prog. Horm. Res. 26, 589-622.

Cotten, M., et al., (1986). Biochemistry 25, 5063-5069.

Crevel, G., etaL, (1997). J StructBiol 118, 9-22.

Dilworth, S., et al., (1987). Cell 51, 1009-1018.

Dimitrov, S., and Wolffe, A. (1996). EMBO Journal 15, 5897-5906.

Dingwall, C., et al., EMBO J 6, 69-74.

Dong, J., et al., (1996). Nature 383, 531-535.

Dube, J. L., et al., (1998). Molecular Endocrinology 12, 1809-1817.

Earnshaw, W., et al., (1980). Cell 21, 373-383.

El-Fouly, M. A., et al., 1970. Endocrinology 87, 288-293.

Elvin, J. A., and Matzuk, M. M. (1998). Reviews of Reproduction 3, 183-195.

Elvin, J. A., et al., (1999). Mol Endocrinol 13, 1018-34.

Elvin, J. A., et al., (2000). Mol Cell Endocrinol 159, 1-5.

Elvin, J. A., et al., 1999B. Mol. Endocrinol. 13, 1035-1048.

Elvin, J. A., et al., 2000. Proc. Natl. Acad. Sci. USA, 97: 10288-10293.

Hogan, B., et al., (1994). Manipulating the mouse embryo-a laboratory manual.

Howell C Y et al., Cell 104:829-38.

Ito, T., Tyler, et al., (1996). J Biol Chem 271, 25041-8.

Iwata, K., et al., (1999). Int J Biol Macromol 26, 95-101.

Krobne, G., and Franke, W. (1980a). Proc Natl Acad Sci 77, 1034-1038.

Kumar, T. (1994). Human Rep 9, 578-585.

Kumar, T. R., et al., (1997). Nature Genetics 15, 201-204.

Laskey, R., et al., (1993). Philos Trans R Soc Lond B Biol Sci 339, 263-269.

Leno, G., et al., (1996). J Biol Chem 271, 7253-7256.

MacArthur, C. A., and Shacldeford, G. (1997a). Genomics 42, 137-40.

Maeda Y et al., Zygote 6:39-45

Maeda, Y., et al., (1998). Zygote 6, 39-45.

Mabmoudi, M., and Lin, V. K. (1989). Biotechniques 7, 331-332.

Matzuk, et al., (1992). Nature 360, 313-319.

Matzuk, M. M., et al., (1995). Nature 374, 356-360.

Matzuk, M. M., et al., (1996). Recent Prog Horm Res 51, 123-54.

McGrath, S. A., et al., (1995). Molecular Endocrinology 9, 131-136.

McLay, D., and Clarke, H. (1997). Dev Biol 186, 73-84.

Meric, F., Matsumoto, et al., (1997). J Biol Chem 272, 12840-12846.

Mills, A., et al., (1980). J Mol Biol 139, 561-568.

Nishimori, K., and Matzuk, M. M. (1996). Reviews of Reproduction 1, 203-212.

Ohsumi, K., and Katagiri, C. (1991). Dev Biol 148, 295-305.

Pedersen, T., and Peters, H. (1968). Joumal of Reproduction and Fertility 17, 555-557.

Perreault, S. (1992). Mutat Res 296, 43-55.

Philpott, A., and Leno, G. (1992). Cell 69, 759-767.

Philpott, A., et al., (1991). Cell 65, 569-578.

Robbins, J., et al., (1991). Cell 64, 615-623.

Schmidt-Zachmann, M. S., and Franke, W. W. (1988). Chromosoma 96, 417-26.

Sealy, L., et al., Biochemistry 25, 3064-3072.

Senapathy, P., et al., Methods Enzymol 183, 252-278.

Service, R. (1996). Science 272, 1258.

Tong Z B et al., Nat Genet 26:267-8.

Vancurova, I., et al., (1995). J Cell Sci 108, 779-787.

Vanderhyden, B. C., et al., (1993) Endocrinology 133, 423-426.

Zuccotti M, et al., J Endocrinol Invest 23:623-9.

What is claimed is:

1. An isolated polynucleotide sequence comprising a nucleic acid sequence of SEQ.ID.NO:11.

2. An isolated polynucleotide sequence comprising a nucleic acid sequence of SEQ.ID.NO:13.

3. A pharmaceutical composition comprising a modulator of O1-180 expression dispersed in a pharmaceutically acceptable carrier.

4. The composition of sentence 3, wherein the modulator suppresses transcription of an O1-180 gene.

5. The composition of sentence 3, wherein the modulator enhances transcription of an O1-180 gene.

6. The composition of sentence 3, wherein the modulator is a polypeptide.

7. The composition of sentence 3, wherein the modulator is a small molecule.

8. The composition of sentence 3, wherein the modulator is a polynucleotide sequence.

9. The composition of sentence 9, wherein the polynucleotide sequence is DNA or RNA.

10. The composition of sentence 9 further comprising an expression vector, wherein the expression vector comprises a promoter and the polynucleotide sequence, operatively linked.

11. A pharmaceutical composition comprising a modulator of O1-180 activity dispersed in a pharmaceutically acceptable carrier.

12. The composition of sentence 11, wherein the composition inhibits O1-180 activity.

13. The composition of sentence 11, wherein the composition stimulates O1-180 activity.

14. A method of modulating contraception comprising administering to an animal an effective amount of a modulator of O1-180 activity dispersed in a pharmacologically acceptable carrier, wherein said amount is capable of decreasing conception.

15. The method of sentence 14, wherein the animal is female.

16. The method of sentence 14, wherein the animal is male.

17. A method of enhancing fertility comprising administering to an animal an effective amount of a modulator of O1-180 activity dispersed in a pharmacologically acceptable carrier, wherein said amount is capable of increasing conception.

18. A method of screening for a modulator of O1-180 activity comprising the steps of:
providing a cell expressing an O1-180 polypeptide
contacting said cell with a candidate modulator;
measuring O1-180 expression; and
comparing said O1-180 expression in the presence of said candidate modulator with the expression of O1-180 expression in the absence of said candidate modulator; wherein a difference in the expression of O1-180 in the presence of said candidate modulator, as compared with the expression of O1-180 in the absence of said candidate modulator, identifies said candidate modulator as a modulator of O1-180 expression.

19. A method of identifying compounds that modulate the activity of O1-180 comprising the steps of:
obtaining an isolated O1-180 polypeptide or functional equivalent thereof;
admixing the O1-180 polypeptide or functional equivalent thereof with a candidate compound;
and measuring an effect of said candidate compound on the activity of O1-180.

20. A method of screening for a compound which modulates the activity of O1-180 comprising:
exposing O1-180 or a O1-180 binding fragment thereof to a candidate compound; and
determining whether said compound binds to O1-180 or the O1-180 binding partner thereof; and
further determining whether said compound modulates O1-180 or the O1-180 interaction with a binding partner.

21. A method of screening for an interactive compound which binds with O1-180 comprising:
exposing a O1-180 protein, or a fragment thereof to a compound; and
determining whether said compound bound to the O1-180.

22. A method of identifying a compound that effects O1-180 activity comprising
(a) providing a group of transgenic animals having (1) a regulatable one or more O1-180 protein genes, (2) a knock-out of one or more O1-180 protein genes, or (3) a knock-in of one or more O1-180 protein genes;
(b) providing a second group of control animals respectively for the group of transgenic animals in step (a); and
(c) exposing the transgenic animal group and control animal group to a potential O1-180-modulating compounds; and
(d) comparing the transgenic animal group and the control animal group and determining the effect of the compound on one or more proteins related to infertility or fertility in the transgenic animals as compared to the control animals.

23. A method of detecting a binding interaction of a first peptide and a second peptide of a peptide binding pair, comprising:
(i) culturing at least one eukaryotic cell under conditions suitable to detect the selected phenotype; wherein the cell comprises;
a) a nucleotide sequence encoding a first heterologous fusion protein comprising the first peptide or a segment thereof joined to a transcriptional activation protein DNA binding domain;
b) a nucleotide sequence encoding a second heterologous fusion protein comprising the second peptide or a segment thereof joined to a transcriptional activation protein transcriptional activation domain;
wherein binding of the first peptide or segment thereof and the second peptide or segment thereof reconstitutes a transcriptional activation protein; and
c) a reporter element activated under positive transcriptional control of the reconstituted transcriptional activation protein, wherein expression of the reporter element produces a selected phenotype;
(ii) detecting the binding interaction of the peptide binding pair by determining the level of the expression of the reporter element which produces the selected phenotype;
wherein said first or second peptide is an O1-180 peptide and the other peptide is a test peptide, preferably selected peptides/proteins present in the ovary.

24. A rescue screen for detecting the binding interaction of a first peptide and a second peptide of a peptide binding pair, comprising:
(i) culturing at least one eukaryotic cell under conditions to detect a selected phenotype or the absence of such phenotype, wherein the cell comprises;
a) a nucleotide sequence encoding a first heterologous fusion protein comprising the first peptide or a segment thereof joined to a DNA binding domain of a transcriptional activation protein;
b) a nucleotide sequence encoding a second heterologous fusion protein comprising the second peptide or a segment thereof joined to a transcriptional activation domain of a transcriptional activation protein;
wherein binding of the first peptide or segment thereof and the second peptide or segment thereof reconstitutes a transcriptional activation protein; and
c) a reporter element activated under positive transcriptional control of the reconstituted transcriptional activation protein, wherein expression of the reporter element prevents exhibition of a selected phenotype;
(ii) detecting the ability of the test peptide to interact with O1-180 by determining whether the test peptide affects the expression of the reporter element which prevents exhibition of the selected phenotype,
wherein said first or second peptide is an O1-180 peptide and the other peptide is a test peptide, preferably selected peptides/proteins present in the ovary.

25. A method of identifying binding partners for O1-180 comprising the steps of:
exposing the protein to a potential binding partner; and
determining if the potential binding partner binds to O1-180.

26. A pharmaceutical composition comprising a modulator of O1-236 expression dispersed in a pharmaceutically acceptable carrier.

27. The composition of sentence 26, wherein the modulator suppresses transcription of an O1-236 gene.

28. The composition of sentence 26, wherein the modulator enhances transcription of an O1-236 gene.

29. The composition of sentence 26, wherein the modulator is a polypeptide.

30. The composition of sentence 26, wherein the modulator is a small molecule.

31. The composition of sentence 26, wherein the modulator is a polynucleotide sequence.

32. The composition of sentence 31, wherein the polynucleotide sequence is DNA or RNA.

33. The composition of sentence 31 further comprising an expression vector, wherein the expression vector comprises a promoter and the polynucleotide sequence, operatively linked.

34. A pharmaceutical composition comprising a modulator of O1-236 activity dispersed in a pharmaceutically acceptable carrier.

35. The composition of sentence 34, wherein the composition inhibits O1-236 activity.

36. The composition of sentence 34, wherein the composition stimulates O1-236 activity.

37. A method of modulating contraception comprising administering to an animal an effective amount of a modulator of O1-236 activity dispersed in a pharmacologically acceptable carrier, wherein said amount is capable of decreasing conception.

38. The method of sentence 37, wherein the animal is female.

39. The method of sentence 37, wherein the animal is male.

40. A method of enhancing fertility comprising administering to an animal an effective amount of a modulator of O1-236 activity dispersed in a pharmacologically acceptable carrier, wherein said amount is capable of increasing conception.

41. A method of screening for a modulator of O1-236 activity comprising the steps of:
providing a cell expressing an O1-236 polypeptide
contacting said cell with a candidate modulator;
measuring O1-236 expression; and
comparing said O1-236 expression in the presence of said candidate modulator with the expression of O1-236 expression in the absence of said candidate modulator; wherein a difference in the expression of O1-236 in the presence of said candidate modulator, as compared with the expression of O1-236 in the absence of said candidate modulator, identifies said candidate modulator as a modulator of O1-236 expression.

42. A method of identifying compounds that modulate the activity of O1-236 comprising the steps of:
obtaining an isolated O1-236 polypeptide or functional equivalent thereof;
admixing the O1-236 polypeptide or functional equivalent thereof with a candidate compound;
and measuring an effect of said candidate compound on the activity of O1-236.

43. A method of screening for a compound which modulates the activity of O1-236 comprising:
exposing O1-236 or a O1-236 binding fragment thereof to a candidate compound; and
determining whether said compound binds to O1-236 or the O1-236 binding partner thereof; and
further determining whether said compound modulates O1-236 or the O1-236 interaction with a binding partner.

44. A method of screening for an interactive compound which binds with O1-236 comprising:
exposing a O1-236 protein, or a fragment thereof to a compound; and
determining whether said compound bound to the O1-236.

45. A method of identifying a compound that effects O1-236 activity comprising
(a) providing a group of transgenic animals having (1) a regulatable one or more O1-180 protein genes, (2) a knock-out of one or more O1-236 protein genes, or (3) a knock-in of one or more O1-236 protein genes;
(b) providing a second group of control animals respectively for the group of transgenic animals in step (a); and
(c) exposing the transgenic animal group and control animal group to a potential O1-236-modulating compounds; and
(d) comparing the transgenic animal group and the control animal group and determining the effect of the compound on one or more proteins related to infertility or fertility in the transgenic animals as compared to the control animals.

46. A method of detecting a binding interaction of a first peptide and a second peptide of a peptide binding pair, comprising:
(i) culturing at least one eukaryotic cell under conditions suitable to detect the selected phenotype; wherein the cell comprises;
a) a nucleotide sequence encoding a first heterologous fusion protein comprising the first peptide or a segment thereof joined to a transcriptional activation protein DNA binding domain;
b) a nucleotide sequence encoding a second heterologous fusion protein comprising the second peptide or a segment thereof joined to a transcriptional activation protein transcriptional activation domain;
wherein binding of the first peptide or segment thereof and the second peptide or segment thereof reconstitutes a transcriptional activation protein; and
c) a reporter element activated under positive transcriptional control of the reconstituted transcriptional activation protein, wherein expression of the reporter element produces a selected phenotype;
(ii) detecting the binding interaction of the peptide binding pair by determining the level of the expression of the reporter element which produces the selected phenotype;
wherein said first or second peptide is an O1-236 peptide and the other peptide is a test peptide, preferably selected peptides/proteins present in the ovary.

47. A rescue screen for detecting thee binding interaction of a first peptide and a second peptide of a peptide binding pair, comprising:
(i) culturing at least one yeast cell under conditions to detect a selected phenotype or the absence of such phenotype, wherein the yeast cell comprises;
a) a nucleotide sequence encoding a first heterologous fusion protein comprising the first peptide or a segment thereof joined to a DNA binding domain of a transcriptional activation protein;
b) a nucleotide sequence encoding a second heterologous fusion protein comprising the second peptide or a segment thereof joined to a transcriptional activation domain of a transcriptional activation protein;
wherein binding of the first peptide or segment thereof and the second peptide or segment thereof reconstitutes a transcriptional activation protein; and
c) a reporter element activated under positive transcriptional control of the reconstituted transcriptional activation protein, wherein expression of the reporter element prevents exhibition of a selected phenotype;
(ii) detecting the ability of the test peptide to interact with O1-236 by determining whether the test peptide affects the expression of the reporter element which prevents exhibition of the selected phenotype,
wherein said first or second peptide is an O1-236 peptide and the other peptide is a test peptide, preferably selected peptides/proteins present in the ovary.

48. A method of identifying binding partners for O1-236 comprising the steps of:
exposing the protein to a potential binding partner; and
determining if the potential binding partner binds to O1-236.


SEQUENCE LISTING (23 sequences)

SEQ ID NO: 1 - DNA, 1258 bp, Mus musculus
ggcgggcgacgcgcgggacgcacccatgttcccggcgagcacgttccacccctgcccgcatccttatccgcaggccaccaaagccggggatggctggaggttcggagccaggggctgccgacccgcgcccccctccttcctccccggctacagacagctcatggccgcggagtacgtcgaccgccaccagcgggcacagctcatggccctgctgtcgcggatgggtccccggtcggtcagcagccgtgacgctgcggtgcaggtgaacccgcgccgcgacgcctcggtgcagtgttcactcgggcgccgcacgctgcagcctgcagggtgccgagccagccccgacgcccgatcgggttcctgtcaaccccgtggccacgccggcgccgggagatccccgcgatcctggcagaccgtagccccgttctcgtccgtgaccttctgtggcctctcctcctcactggaggttgcgggaggcaggcagacacccacgaagggagaggggagcccggcatcctcggggacccgggaaccggagccgagagaggtggccgcgaggaaagcggtcccccagccgcgaagcgaggagggcgatgttcaggctgcagggcaggccgggtgggagcagcagccaccaccggaggaccggaacagtgtggcggcgatgcagtctgagcctgggagcgaggagccatgtcctgccgcagagatggctcaggaccccggtgattcggatgcccctcgagaccaggcctccccgcaaagcacggagcaggacaaggagcgcctgcgtttccagttcttagagcagaagtacggctactatcactgcaaggactgcaaaatccggtgggagagcgcctatgtgtggtgtgtgcagggcaccagtaaggtgtacttcaaacagttctgccgagtgtgtgagaaatcctacaacccttacagagtggaggacatcacctgtcaaagttgtaaaagaactagatgtgcctgcccagtcagacttcgccacgtggaccctaaacgcccccatcggcaagacttgtgtgggagatgcaaggacaaacgcctgtcctgcgacagcaccttcagcttcaaatacatcatttagtgagagtcgaaaacgtttctgctagatggggctaatggaatggacaagtgacgtttctcccctcttcacctcttccctttccaaattcttcatgacagacagtattacttgagtataaagcctgtgaataaaaggtattgcaaacaa

SEQ ID NO: 2 - protein, 361 aa, Mus musculus
MFPASTFHPCPHPYPQATKAGDGWRFGARGCRPAPPSFLPGYRQLMAAEYVDSHQRAQLMALLSRMGPRSVSSRDAAVQVNPRRDASVQCSLGRRTLQPAGCRASPDARSGSCQPRGHAGAGRSPRSWQTVAPFSSVTFCGLSSSLEVAGGRQTPTKGEGSPASSGTREPEPREVAARKAVPQPRSEEGDVQAAGQAGWEQQPPPEDRNSVAAMQSEPGSEEPCPAAEMAQDPGDSDAPRDQASPQSTEQDKERLRFQFLEQKYGYYHCKDCKIRWESAYVWCVQGTSKVYFKQFCRVCEKSYNPYRVEDITCQSCKRTRCACPVRFRHVDPKRPHRQDLCGRCKDKRLSCDSTFSFKYII

SEQ ID NO: 3 - DNA, 1817 bp, Mus musculus
gtcacagctttcccctgcccgaatatggtgatctgtctccattgtccagatcaggatgattctttagaagaagtcacagaggaatgctattccccacccaccctccagaacctggcaattcagagtctactgagggatgaggccttggccatttctgctctcacggacctgccccagagtctgttcccagtaatttttgaggaggccttcactgatggatatatagggatcttgaaggccatgatacctgtgtggcccttcccatacctttctttaggaaagcagataaataattgcaacctggagactttgaaggctatgcttgagggactagatatactgcttgcacaaaaggttcaaaccagtaggtgcaaactcagagtaattaattggagagaagatgacttgaagatatgggctggatcccatgaaggtgaaggcttaccagatttcaggacagagaagcagccaattgagaacagtgctggctgtgaggtgaagaaagaattgaaggtgacgactgaagtccttcgcatgaagggcagacttgatgaatctaccacatacttgttgcagtgggcccagcagagaaaagattctattcatctattctgtagaaagctactaattgaaggcttaaccaaagcctcagtgatagaaatcttcaaaactgtacacgcagactgtatacaggagcttatcctaagatgtatctgcatagaagagttggcttttcttaatccctacctgaaactgatgaaaagtcttttcacactcacactagatcacatcataggtaccttcagtttgggtgattctgaaaagcttgatgaggagacaatattcagcttgatttctcaacttcccacactccactgtctccagaaactctatgtaaatgatgtcccttttataaaaggcaacctgaaagaatacctcaggtgcctgaaaaagcccttggagacactttgcatcagtaactgtgacctctcacagtcagacttggattgcctgccctattgcctgaatatttgtgaactcaaacatctgcatattagtgatatatatttatgtgatttactccttgagcctcttggttttctccttgagagagttggagataccctgaaaaccctggaattggattcatgttgtatagtggactttcagttcagtgccttgctgcctgccctaagccaatgttctcacctcagagaggtcactttctatgataatgatgtttctctgcctttcttgaaaacaacttctacaccacacagccctgctgagtcagctgatctatgagtgttaccctgcccctctagagtgctatgatgacagtggtgtaatactaacacacagattagaaagtttttgtcctgagcttctggatatactgagagccaaaagacagctccatagtgtctcctttcaaacaaccaaatgctctaaatgtggtgggtgctacatttatgatcggcatacccaatgttgccgttttgtggaactactataagcttgattgtgaaactgagaaatagaaacttagtattggggactgatgaaatcctaagtgaatgtccactgctaaatggagcatgaaaatgtcaatcacctaaaagtctgagatacacaggaaagtcaataacttcctctgagctggtgaatggatgttgcatctgtagaaagtatcaagcacttgtagtttgaatgtgttacaatagaagcaccattttatgagactggcccaatctgttgactgcatacaataaatctgttgacttattaaatttttaaaaaaaaaaaaaaaaaaaaaa

SEQ ID NO: 4 - protein, 426 aa, Mus musculus
MVICLHCPDQDDSLEEVTEECYSPPTLQNLAIQSLLRDEALAISALTDLPQSLFPVIFEEAFTDGYIGILKAMIPVWPFPYLSLGKQINNCNLETLKAMLEGLDILLAQKVQTSRCKLRVINWREDDLKIWAGSHEGEGLPDFRTEKQPIENSAGCEVKKELKVTTEVLRMKGRLDESTTYLLQWAQQRKDSIHLFCRKLLIEGLTKASVIEIFKTVHADCIQELILRCICIEELAFLNPYLKLMKSLFTLTLDHIIGTFSLGDSEKLDEETIFSLISQLPTLHCLQKLYVNDVPFIKGNLKEYLRCLKKPLETLCISNCDLSQSDLDCLPYCLNICELKHLHISDIYLCDLLLEPLGFLLERVGDTLKTLELDSCCIVDFQFSALLPALSQCSHLREVTFYDNDVSLPFLKTTSTPHSPAESADL

SEQ ID NO: 5 - DNA, 1018 bp, Mus musculus
gccatattgaggacctgcagtagaggtggaacccatgactggcagcgcaaacacagtgataacagctgagctccaagcaaggacccaggaccttgcctcaccacagacataatctttccccacaacacctccaccaagccgccctgtaaatcgacatgagtcgccacagcaccagcagcgtgaccgaaaccacagcaaaaaacatgctctggggtagtgaactcaatcaggaaaagcagacttgcacctttagaggccaaggcgagaagaaggacagctgtaaactcttgctcagcacgatctgcctgggggagaaagccaaagaggaggtgaaccgtgtggaagtcctctcccaggaaggcagaaaaccaccaatcactattgctacgctgaaggcatcagtcctgcccatggtcactgtgtcaggtatagagctttctcctccagtaacttttcggctcaggactggctcaggacctgtgttcctcagtggcctggaatgttatgagacttcggacctgacctgggaagatgacgaggaagaggaggaagaggaggaggaagaggatgaagatgaggatgcagatatatcgctagaggagatacctgtcaaacaagtcaaaagggtggctccccagaagcagatgagcatagcaaagaaaaagaaggtggaaaaagaagaggatgaaacagtagtgaggcccagccctcaggacaagagtccctggaagaaggagaaatctacacccagagcaaagaagccagtgaccaagaaatgacctcatcttagcatcttctgcgtccaaggcaggatgtccagcagctgtgttttggtgcaggtgtccagccccaccaccctagtctgaatgtaataaggtggtgtggctgtaaccctgtaacccagccctccagtttccggaggtttttggtgaagagcccccagcaagttcgcctagggccacaataaaatttgcatgatcaggaaaaaaaaaaaaaaaaaaaaaaaaaaaaaaaaaaa

SEQ ID NO: 6 - protein, 207 aa, Mus musculus
MSRHSTSSVTETTAKNMLWGSELNQEKQTCTFRGQGEKKDSCKLLLSTICLGEKAKEEVNRVEVLSQEGRKPPITIATLKASVLPMVTVSGIELSPPVTFRLRTGSGPVFLSGLECYETSDLTWEDDEEEEEEEEEEDEDEDADISLEEIPVKQVKRVAPQKQMSIAKKKKVEKEEDETVVRPSPQDKSPWKKEKSTPRAKKPVTKK

SEQ ID NO: 7 - DNA, 6970 bp, Mus musculus
  misc_feature (1)..(6970): N equals unknown
acagcagaggtgatgctcagaaatcaagttttaacagagggccaggtgcttctagagtaggaggggattgcacacctccccaccccctcctctttcccaggcttcttaacagcctgctgtgggaagctgacccttagatggagccctgaagccatattgaggacctgcagtagaggtggaacccatgactggcagcgcagtaagcttgagcaggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctttgcattactcagaacacagtgataacagctgagctccaagcaaggacccaggaccttgcctcaccacagacataatctttccccacaacacctccaccaagccgccctgtaaatcgacatgagtcgccacagcaccagcagcgtgaccgaaaccacagcaaaaaacatgctctggggtaagggctaaggctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtcttcgctgtgcaggtagtgaactcaatcaggaaaagcagacttgcacctttagaggccaatgcgagaagaaggacagctgtaaactcttgctcagcacggtgggtgtctcccaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncatcacctttctcagatctgcctgggggagaaagccaaagaggaggtgaaccgtgtggaagtcctctcccaggaaggcagaaaaccaccaatcactattgctacgctgaaggcatcagtcctgcccatggtgagtcttctctccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagaagggggacacaggtcactgtgtcaggtatagagctttctcctccagtaacttttcggctcaggactggctcaggacctgtgttcctcagtggcctggaatgttatggtaagttgtagcctannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggctacccattccagagacttcggacctgacctgggaagatgacgaggaagaggaggaagaggaggaggaagaggatgaagatgaggatgcagatatatcgctagaggagatacctgtcaaacaagtcaaaagggtggctccccagaagcagatgagcatagcaaaggtggggggaaaagaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntggtttttgttccagaaaaagaaggtggaaaaagaagaggatgaaacagtagtgaggtaattcatgcagttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctattccctttccaggcccagccctcaggacaagagtccctggaagaaggtgagcaataagaagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctcttatctgcacaggagaaatctacacccagagcaaagaagccagtgaccaagaaatgacctcatcttagcatcttctgcgtccaaggcaggatgtccagcagctgtgttctggtgcaggtgtccagccccaccaccctagtctgaatgtaataaggtggtgtggctgtaaccctgtaacccagccctccagtttccggaggtttttggtgaagagcccccagcaagttcgcctagggccacaataaaatttgcatgatcaggacctccctctgcctccccctccctggatgggtctcctcgctgctgcgatagctcatgtgcccagcagagggcaaccacgagcaagaaaccagccccatgt

SEQ ID NO: 8 - DNA, 1207 bp, Human
agggggcgccaggaggcctcggcgggtccgcaattggccgggacagcttctcacgaaaggtcctgggccggcatcatcagcctcacctgggaactggttagaactacaaattccctcggccccacccagaccgacgccaagggcagctgtggagtggggcgcggcaatgcgccccttaacagccctccaggcttcttagcccgggcttggacagccgccttccggccagaggggatgaggttgcgctgcgctccgggagcgccgatggcgtgactggccccgcgcggagcagcgacactgcccggccagcccgcttctctgcccggagccatgaatctcagtagcgccagtagcacgaggaaaaggcagtgacgaccgtgctctggggctgcgagctcagtcaggagaggcggacttggaccttcagaccccagctggaggggaagcagagctgcaggctgttgcttcatacgatttgcttgggggagaaagccaaagaggagatgcatcgcgtggagatcctgcccccagcaaaccaggaggacaagaagatgcagccggtcaccattgcctcactccaggcctcagtcctccccatggtctccatggtaggagtgcagctttctcccccagttactttccagctccgggctggctcaggacccgtgttcctcagtggccaggaacgttatgaagcatcagacctaacctgggaggaggaggaggaagaagaaggggaggaggaggaagaggaagaggaagatgatgaggatgaggatgcagatatatctctggaggagcaaagccctgtcaaacaagtcaaaaggctggtgccccagaagcaggcgagcgtggctaagaaaaaaaagctggaaaaagaagaagaggaaataagagccagcgttagagacaagagccctgtgaaaaaggccaaagccacagccagagccaagaagccaggattcaagaaatgaggagccacgccttggggggcacggtgcaaagtgggccttccctgggctgtgctgcaggcacagggtgcccctgtccagcccctccacctgtgtctgaatgcaacaggggtgttgcgggggcaacatgagagcccctcacccccaactctccactttcaggaggcccccagtgaagagccccacctcggggtcacaataaagttgcctggtcaggaaaaaaaaaaaaaaaaaaa

SEQ ID NO: 9 - protein, 214 aa, Human
MNLSSASSTEEKAVTTVLWGCELSQERRTWTFRPQLEGKQSCRLLLHTICLGEKAKEEMHRVEILPPANQEDKKMQPVTIASLQASVLPMVSMVGVQLSPPVTFQLRAGSGPVFLSGQERYEASDLTWEEEEEEEGEEEEEEEEDDEDEDADISLEEQSPVKQVKRLVPQKQASVAKKKKLEKEEEEIRASVRDKSPVKKAKATARAKKPGFKK

SEQ ID NO: 10 - DNA, 19951 bp, Human
ggcatccccatatgatggttactagcggctgggaagtgggggtggggggaggatgaacagaggttgattaatgggtacaaacatactgtttgatggaaataagataagttgaaataagaaataagttgatagtacagtagggtgactatagttaacaataatttattgtatatttcaaaatagctagaagagaagatttgaaatgtttccaacacaaagaaatgataaatgtttagccgggcccagtggctcatgcctgtaatcccagcactttgggaggcctaggcaggaggatcactgaggtcaggagttcgagaccaacctggccaacatggtgaaaccccatctctagtaaaaatatgaaaattagctgggcatggtggtaagcacctataaacccagctacttgggaggctgaggcaggagaatcgcttgaacatgggaggcagaggttatagtgagctgagatggcaccaccgcactccagcctgggtgatgagagtgaaacgccatctccaaaaaaaaaaaaaaaaagaaaagaaataatgttgaaggtaccccagttaccctgatttgctcattacacattgtgtgcaagtataaaaatatcatatgtaccccataaatatgtacaattaatatgtatcagtttaaaaagttaatgacgatggtaccaatattatagcctcatttagcagatgaggaaactgaggcactgagctatgaattaacacacctgaaatcacagagcacagtccagacttgaacccagactgtccagttccagtgtcccagctctaggtcatgacctcagggtcatccccctcccctgcctccatttagccttcactgtgacccccagctgcagcctgacatcagtgtgattattcacggggtgggtagcctggggccactgaaggccggtttgctttgagcactgctctccaatgaggctggaagccctttgaggggctgtcgtattcaccgcgggatgcccaggtcccgcccaattggcggaatcaccgtttgttgagtgaattcttgaacgtctgtgcatggcatgcatgtgcctgccatttgctcatctttaccacacacttggctgcaatggctgtcaccttcaataggcgccttgccagggacagagagctcagggacagccaaggagactggaactgctcagctggggatagggagcctagggggccctggcaggcccccaagcctctcctgggttgtcctggccaattcacagggaggctgaccccagcttccaggaataaaagattctgacctctccgtggcaatgagcccctgtcccaggggtgtgcgggagatccctggttatctgaggtgtctccagggtatatccgctgaaaccccacttcctcttcactgcccactgagcctgggaccagctgctggtcacgtgcttggccctgtagcgtcgctagccgtgctcctcagttgtgctccccgccccctgccgcggcgcctcgcctcccggctcacctccccaccccacctgcccgctgcggctctccggcgggagatctcaccgttctggagacagggctcgctcgctctcacggtaggctggaagaacgggctgtctgggccttaggaaaggcccatgctgtataaggcatggggaaaggaaaggaagaaaggcaacgaacaagaaggagggcttccaactgcagcttcctgccggctgcaggcctcccttcctaagctgagctgaggcttcctctccatgggctggggagggggcgccaggaggcctcggcgggtccgcaattggccgggacagcttctcacgaaaggtcctgggccggcatcatcagcctcacctgggaactggttagaactacaaattccctcggccccacccagaccgacgccaagggcagctgtggagtggggcgcggcaatgcgccccttaacagccctccaggtgattctgccgcgcagaggaggaaagaatgggagaagggaaggggagagggaggcggcttctttgcgactaattggacacctgcccttccccttcccaggcttcttagcccgggcttggacagccgccttccggccagaggggatgaggttgcgctgcgctccgggagcgccgatggcgtgactggccccgcgcggagcagcgacagtaaggctgtgtggggggagctgggacctaagccgcgcgcacacccctttctctgcgtctggtggaggtgcacagaggcttttgagtcaggcccaagcgcagccaggtgacctccccgcggcctttcaagcctgagctcggtggacagctccctctcccgtgagtcccgctgtcctgtacgcgcccggtcgagccccgggctgcgcaccccgctaggaggtgggtactcgtcctccaggagttgccggtgagcccttgaccgtggcaggtcccctccagccgcgagcgacccctcagtacctgccgatgcctgctggtctctggcatcctccagtcgagggtcagggtcagggagcaaggcctcacgcgggcgccctccttgcagctgcccggccagcccgcttctctgcccggagccatgaatctcagtagcgccagtagcacggaggaaaaggcagtgacgaccgtgctctggggtgagtggggactcaggctccttcccagagacacgccccacctccggtgcgcggcagcttggggcgcaggtgagcccctcctttgggaacgaatggagggccccacttccctccctttctcctccgcaggctgcgagctcagtcaggagaggcggacttggaccttcagaccccagctggaggggaagcagagctgcaggctgttgcttcatacggtaggtgttcccaaaagaggggaggaagatggtgtccgggaactttctggtcccaacggagggctatggatttctcccgtcggccctcagggtgatgaggggcctctattttcaaccccgctcagatttgcttgggggagaaagccaaagaggagatgcatcgcgtggagatcctgcccccagcaaaccaggaggacaagaagatgcagccggtcaccattgcctcactccaggcctcagtcctccccatggtgcgcatttccctgctggctggaagactgctgtcagcctcaccctcacccttgggtggggatggacacacacgagggtgcattcaccctacagaaatgagccatgctagggaggtagcacagcccatgcagaaagccggggtccagcccagctcaccccttcctagagctgtgtgaccttgggccagttaagctgtctgcaaaaattacactttaaaccaggggtccccaactgccgggtcaaggaccagaactcgtcctgttaggaaccgggcagcacagcaggaggtgagcagccagctagccagcattacccctgagccctgcctcccgtcagatcagagcggcattagattctcagaggagcatgaaccctatggtaagctgtgcgtgcgaggggtctaggttgcatgcttcttatgagaatcgaatgcctgatctgaggtggagcagtttcatcccaaaaccaccccccactcccaccccatccatgcaaaaattgtcttccaggaaaccattccctggttccacaatgattggagactgctgctttaaaccattcactgctagtgacaggaacatggtcgatctacatattggttgtgcatccagaaattctctagtttctaataacttaagttttctatgcatataataattgtgaataatggcagttcttgccttttttttttttttttttgagatggagtttcactctgtcacccaggctggagtgcagtggtgcaatctcagctcactgcaatttctgcctcccaggttcaagcaattctagtgcctcagccttcggagtagctgggattacaggtgtgcaccaccatgcctggctaatttttgtatttttagtagagacagggtttcacaatgttgcccaggctagtctcgaactcctgacctcaggtgattcgcccgccttggcctcccgaagtgctgggattacaggcgtgagtcaccacgccaggtcttttttctttctttctttcttttaatcctactgcactggctaggccctgcagtggaatattgggtgaacatgagatcaggtctgacatccttatttccttcttgttttttaaaaaagaagcatttggtaagtacattttgtcaggttaaggaagtccccatctaaaaccctctgcttttaaaaaattgctttgctttgaaatcactagagggggttaaattttaccaaatgcttttctattcatatgattctaggttctttagtccacgtggtaaattgtatgaagagaataatatctgagttcctttgccatcctgggataaatactactggtcatgttacaaattctagatttgttttactcatatattgagcccttttttttacctgtatacatgtgaggttggcctataagtgatagtatttaggtgttcaggacatgctagcctcatgcactgagggtggagggttctctcctattccctggaacagtgaatggaagactaggatcagctgtccttggaggtttgggagacccctctgtcaaactgccctcagactttcctgttctatttacacatactttgcaggcgatctcatcctttcctgtggttttcaagaccatctaaacaaatgaagactcaggaacttatttctgtaaactcaacctctcttgagctccaagtcctatatccaactaaatggctttgatagatatctaataaatatcccaaatttaacatgtctaaatccacatattcaatttttatctctaatccacccgcctccccttcaacctgattttctctcagaaaacagctgtttttccagttagccaagacaaagctctttttttttttttttcccctgagatggagtttcactcttatcacccaggctggagtgcaatggtgcgctctcggctcactgcaacctccgcatcccatgttcgagcgattctcccatctcagcctcccaagtagctgggattacaggcatgttccactacgccaggctaatttttgtaattttagtagagatgggtttttgccatgttggtcaggctggtctcgaactcctgacctcaggtgatatacccgccttggcctcccaaagtgctggatttacaggcatgagccaccacacccagccaaatcctcttttttcccacacccatatctgatctaccagcagtcctgttgtctctgcccccatcttataccccaatggaccacatctcatcatcttccctgctacccctggtacaggtgacagttgcctgtggctccattttaattgcacagccttccacctggtctacctaccatcacatggtcccctgtagtctattccagggtaggcaaactagagggcttgaatctaggctgctgcctggttttgtaagtagttttactgggaacacagccacactcattcgtttgtaccctgtccatggctgcttttcctccctaacagcagatttgagtagtctccatggagaccatatggtttgcaaacctaaaatattaccttctggctcttaacagaaagtttcctggtctgtgctccacacagctgccaaaaagattttttttttttttttttttttttttgagacagaatcttgctttgataccagggctggaatgcagtggcttaatctcggctcactgcaacctccacctccttagtagccgggacaacaggcgctccccaccatgcccagctgattttttttttttttttgtaatttttagtagagacggggtttcaccatgttggccaggctggtcttgaacttctgacttcaagtgatccacctgcctcagcctcccaaagtgctgagattataggcgtgagccactatgcccagccaaaaagatccttttaaacacaggttagatcatgtggcttctctgctagaatagttaggtcatggctctctctcatttggaataagagccgagagtgtattatggcctgcttcgaagcctttgtgttctggcctcagcaacctctctgtttcaggtgtgttttatgtcttatgttccaggtatgtatcttttacacagtatgtagctagattttgttctatctggccagtatagtctgtattcattgtgattgctgatgtaattggatttgtggcatgtactttgtacccctactttccttgcttttttattttcttctccttttcctatattttattagattaattaaagttccttttccccttctctactggtttggaagttatagaatctcattctaatttttttactgtttatctttaatttttttaacaatcatacattactcaaagtttagaattaatgttttatgtcctcccagacaatccaaggagcttttctgattctcctttttttatttttttattttcgaaatggagtctcactttgtttcccaggctagagtgcagtggtgcaatcttgactcactgcaacctctgcttcccaggtgattcaagtgattctcctgcctcagcctcccaagtagctgggattacaggtatgcaccatcatgcccagctaatttttgtatttttagtagagacagggattcaccacgttggccaggctggtcttgaactcctgagctcaggtgatcctcctgccttggcctcccaaagtgctggaattataggtgtgagctaccgcacctggcctgattctcatctttttaacttcaatattattatcaagtgttctagctccaacttgtcagcctactcaacactactcattactagtactattgtattttccagttaattcttatgtaggtttactttgtttaccaattagtttggttaccactgcttctagcacccacttcttccttcttgatctaatttcttatcctttttagcaacatactttagtaagcctgagaatagtaaacttattcaggctttgtctggaaatatcatgattttgccctcattccttcatgatggtttggctgtgtatataattctagatagagagtttccttcagctttgaagctgttctattctggttcccaatgttgctgttgaagtcctcaatctgtctgattattccctttttggagatgtctttcctctctggcttattttaagataatgtcttttgtttttattttctatagttttaccatgatgtgttcaggtgtagatttattttttttgtctgttcaggacttaggttttcagacatgaggatctatgtctttgatcaattctggaaaattattggttgtttttcctttgaatattgactgtcttccagtctccctagtcccttttccaattagatatatgttgggccttctcactctgtcccccgtgttagctcccccaatgtttctttaaatctctccctctctgccttatactgggtaattccttcacagcattattgatcatactaattctgcttctgctggttttttctgctttttaatcatttgatcaggttttcttttatttttgttttaaagacaaggtcttgctctgttgcctaggttggagtgcagtggcctgatcatggctcactgcagcctcaaactcctgggctcaagtgatcctcccactcagcctcctaagtagctgggactacaggcatgagccaccatgcccagctaatgtttattatttttttgtagagctggagtctcactatgttatccaggctggtttctaatttctgacttcaagcactcctcccacctcagcctctcaaagtgctgggattataggtgtgagccaccatgcccatcctcagctttgggtgttttatatctagaaagctgcatttggttctttttcttttcataatgtcttgttcttatgattatgattcctacttttatatttacttggttttaaacatatacatatatatatatatgtacatatttctgtatatacatattttagagacaagatcttgctctgtcgcccagtctggagtgcattgttgcaatcatagctcactgcagccttgaactcctgagttcaagcgatcttccggcctcagcccccccgagtagcctgggctacaggcgtgcaccaacacacccagctttatctgatatttttttagatcagtgttctaagttcttggtcgggctagtctgcagtcacttgatttctttctgttgactctagctggattgtttcttgtatgttttgtaattttatacggtgagctcatctttagtttattttgtttttttccaaaagaattcgatgtggcctgagtttggggagtgttccaacagagtggtttcgtgtttgcttctgccatttacctcaggaatatcataagcttgggattttctgtacatttcttggcttggcagtactcactgagtaaattcagaccccataagtgagaggtacagctatggggtatgggctctcactggagacttcttttatttccattcatgctttgttgttagcttcctttaatggtggctagggttttgatttttgtttttcatttttgaggagaagagggctggtttgggcttttgagtctctaatcctcaacacttaccttgggcctctcactaagggtatagcccttgagggtcctaccctccccatggtggtctcagctacaactctcctccttgcctgagcccaaggccttgtctcctgactgtgaagattttgttgttgttctgttgtgttttttaaagacagtcttgttctgtcgcccaggctggcacgcagtggcgcaatctcggctcactgcagcctccgcttcctgggttcaagcagttctcctgcctcagccccctgagtagctgggattacaggtgtgcaccatcacacctggctaatttttgtatttttagtagaaacagggtttcaccatgttgcccaggctggtctcaaactcctgacctcaagttatccgcctgccttggcctcccaaagtgcttggattacaggcatgagccaccgtgcgtgctcggcctgtgtgtgagttttgaagcaaaaagtcctggctgttttcaggtccatttcccctcggttgcagcacaccagctcctgcacctgcctgtcttcatttcttttttctttttttcattcatcactaatcagaaggcatcctctcttcattttttatgtacgaggattcttctgtcttactgttcagccatgcagtagaaacactgaattacatcctctctggcatttctaagtgtctctggctgcagagtttgttttcacttattacctcctgtctgaacttagagtttagaagctgtaagttattcactctaagtctcagtttcctcctctgtaaagtatcagtacttacatcatgggtttcttgtgaggatttaatgagataaagcagataaaatgcttagcagggtgcctgacacgtggcagaagctcaaaacaataagctatcattgtcattcgagaaaatttgggaagtttggaaaagtataaatacaataaataccttactatcgactgacaattatggttagcactttaatatattttaaacctttattcttatgtatatccatacattatacataagacaaaagtagtactgtagaggctcttgtcatttataagtgtgatgattggggtttcacgctcgtgtgtaaggtgtgcctcccacaaacctggttacgagttggcacatcacctgtctgatgtgaagaaagcaagcagcactgtacataaaatcatgcatctgctttctcgtttgatcggtgtctcagtctgcccaagttgctataacaaaataccacagactggagggctttagcaacagacgttttctcacacttctggaggctttgaggtctgagatcagggggccggcatggttgggttctggtaagggtcctctccctgagttgcagacggcagccttcttgctatgtgttcacgtgtggggcaggagagtggagagaaagagagtgagttctctagtgtctctttttgtaaaggtactagttctatcatgagggtcctgccctcatgaccccaaacctaatgacctcccaaggcctgcatttctaaatactatcacactggaggatagggcttcaacataggaatttggaggatggggggaatggccataatttagcccatactaatcagattcctctatctgacggctgcccttcagcttggaagcccatctgtgagcttagcaagaatgggattaccaggttcaaattcttggacagaatccacctcagcaggggctgagtagtgtgtagggtttggggagggaaatcaactcagtatttctgcctgaggccagtccccgagaggtgccaggcctaagtggccctgtcttctctgcttcctccccctgcaggtctccatggtaggagtgcagctttctcccccagttactttccagctccgggctggctcaggacccgtgttcctcagtggccaggaacgttatggtaagtcagagcctgcgatcaggaaggtccgtgagtaccgtgctaggcaggggctcgggacatactagctactcaaacactggagggattcttgaatgttggaagaaaatccccaaaggcaacatgacagccagcagcctggactggaaggcaagggcgctggtccctgttcttcttctttacctgccatgacctctgtaggctgcagcccctcacttgtaaacttcaaagagcagttgtgaagaataaatgggatattcaggaaaagcactcagcgtaatacccagcactagggaaccactgttcaggatgtggctgctgcagtgatgcagaccatagcaacgcagaccatagcaacgcagaccatagcaacgcggcatgatgctgactccttcaaggtcccttcaactggccctcttttctgtatgattatgcctcattcatcagggtactctcctgctaaaaagccttggcaggtcccacttctcttaggataaggtttcaattctttagctatttgttttagattctttccccctctcctcctcttcctcccctactctgcctacctttagccttggccccagcccttgccaatatgaatccccctcctacccagccagagccacttcccctgccctctttctaccaccccagccctctgcagggctttcctagaccccctaccctaccctggcctccactgttgggagggccagaaagggtgccgccctgtacaggtggcaggcaggtaaccactgtcaactccaggctaggattcctccagggcagtgcttggagcaacacggatcacagaatgggaggtgggcattgattctgtagctctgaagctgtgcccctgcatcctttcccatgctattacagaagcatcagacctaacctgggaggaggaggaggaagagaaggggaggaggaggaagaggaagaggaagatgatgaggatgaggatgcagatatatctctggaggagcaaagccctgtcaaacaagtcaaaaggctggtgccccagaagcaggcgagcgtggctaaggtgggggaaggagcgtggctgtttggaaggaagtggtacccctacagaagcacttaagaggggtgggccaccgggagcctgggccagcctcccagaatgagtgtacaggatgggccaaggccacctcagctagttctggccaggagctcagcagggaccttgtggactttgggaatctgttgtggctctggactttgtctgaactctcataatacactgttttttggttcccagaaaaaaaagctggaaaaagaagaagaggaaataaggtaactctttctacctattaaattagccaaagtctccagctgagatatacagtgttagaaagaatactgtgctgttgggatgtacgtgtacaaatgtacacacggtgtgtctacctgcactcgcaggcacatgggtatggaagtgctgaagggtggcatcacctttctggaagagcattacaacgttcttatcttgggatctaattccagtgaaggcaattccttccacagaattccatccaaatttcaggggaaattacctgcactaagatgcttctcacggccaggcttggtggcccacgcctgtaatcccagcactttgggaggctaaggcaggcagatcacttgaggtcaggagctcgagactagcctggtcaacatgatgaaaccctgtctctactaaaaacacaaaaattaaccgagcctggtggcattcttgtaatcccagctactcaggaggctgaggcatgagaattgcttgagcccggggaaaagttgcagtgagccgagatcgtgccactgcgctccagcctggatgacagagcgagactcagtctgaaaaaacaaaattttaaaaagacgcttatggcattattcgaatagtgaaaaaatggaagcattctaaatgtctaccaatataacaattcactaagctacatccctcttctcaatggaatattacataatgcttatgaagaatatagcaacctggaaagtatgtgtatagttttggtttgtttgtttaatgagacagggttttgctctgccacccaggctggagtgtgatggcacaatcatggctcactgcagccttagcttcctgggctcaagcaatcctcccacctcagctttccaagtagctaggactataggcacgtgccactatgcatggctaacttttaagttttgtgtagagacagggtctttctatgttgcccaggctgatctcaaactcctgacctcaagcaatcctcctgcctcagcctcccaaagcactggaattacaagtgtgagcctctgcacctgataagaatattgatagttcatacagcaggacataaagtcatttttattttatttcacatatttttaaaaagagtttgaccaggccgggtacggtggctcacgcctgtaatcccagcactttgggaagccaaggtgggaggatcacttgaggtcaggagttcaagaccagccttgccaacatagcaaaaccctgtctctactaaaatacaaaattcagctgggcgtggtggcatgtgcctgtaatcccagctactcgggaggctgaggcaggagaatcacttagacccggaatgcgaaggatgcagtgaaccaagatcacaccactgcacaccagcctgggcaacagagcaagactccatctcaaaaaaaaaaaaaaaagtttgaccaagaaaaaaataataaccctgaaagaaaatacaccaaaatgtttagtgtgggcagtaaagaaaactataagtaatgtattttcttgtctatttgctatattttgtacaaaatggttaatattttataatgaaaaagacatttgtgggccaggtgtggtggtgcacacctgcagtcccagcttctcaggaggctgaggcaggaggatcactcgagcccaggaggtcgaggctgcagtgagctgtgatagtgtcactgcactccagcctgggcaacagaaccagactccatctcaaaaacaaaacaaaacaaaagacatttgtgataactaaatgaagatggaagcctaaggaaaacacatatgcgtatgcatgcacacgcacacacatccctttgtttaaagagtccgagtggtccccaggaggagcagccaggcttgctttccagggtgggcactgggagggccacgccgctggtctggagctgagctctctccctgaccccaatcccactcctgctccgctccaccctgttgcagagccagcgttagagacaagagccctgtgaaaaaggtgagtaggaccagagggctttggcccttgggacaggcgagtattctctggagggggctgcctggtatggagaagggaacgggaccctggagccctgccttccctccacaggccaaagccacagccagagccaagaagccaggattcaagaaatgaggagccacgccttggggggcacggtgcaaagtgggccttccctgggctgtgctgcaggcacagggtgcccctgtccagcccctccacctgtgtctgaatgcaacaggggtgttgcgggggcaacatgagagcccctcacccccaactctccactttcaggaggcccccagtgaagagccccacctcggggtcacaataaagttgcctggtcaggactttccttctcttccctggagccagcctccttgtccgctgcaccagccccagtgcccggcagagggcagccttgaaccggtgcacccgggccctgaggtcatacctgcctccctgcacccagcccccgcggcctgagcctgctgtgtccctcgtcctcggcacccccaattctcccccagtggcgagggaagagcctagagtctgccttctgctgagctgtgtgtcaggtggatttccagcctgcaccctccctctgggcagagctaggtttataggcacccaagggctacggctgctcaagctaccagaaggggcctcgccctagggggccagcccccagggtcttctcctgaccttattcctgtcaggcagctactgtgtgcagagcatctatagggaactcagggacatccgctctccctgcttgcttccgttaggggccagctcatcttatagggacctcccacatgtgaagatctgtgtcaggcaggagccagaggcccgcaccttcaaaaaaacctttgaggtggagtagacagggtgagctttacagaggcgccaagccccacatgctatcgagtaggcctcagtcaagcataggggcgaggccaagagaggactggaaaatggggtgggggacccccaccctctccctggtgtgcagaggggactctggagggctgttcacctgtgggtgaccctggcgcagttcctagaacaggcggacacacagatgagccctacactctgggttatccatgcagcgcctctgctggcttctccctgcccctccccagcaccctctggggtcaggcccgaagtgaaccagtggggagctggtcctgctgtcctgcttagtacccccaggtatggggcccaggaggtcggagctctttgaacacctgcctaggaaagtcaacaaccaggctggggcctcctgtccagctatagcttcttttgagaccagagacagaggtagcagagggcagggttgtattcattttttttttaatttttattttttttagagacagggtctcactctgtcaccgaggctggagggcagtggcacagtcttagctcacggcagcctcgacctcctggactcaagcaatcctcccacctcaacctcccaaagtgctggaactacaggcacgagccaccacaccaaaacaaattttaaaattttttgtagagatggggggtcttgttaagttgcccaggccagtctccaactcctgggctcaagagatcctcctgcctcagcctcccaaaatgctgggattacagacgtcagccactgcacccacccagggtggcgttctccctgcatgtttcctacacccatgagacagatgtgggtgctgtcctgccctccaacagacaagccactaactttaggtcacccagagtcccaccctccaacaaagggacaaaccactaacttcaggtcacccagagagtgacaagggggactgctccatgtgagcagctggtgctttttgaacttggtttcatctacagtgacccggggtaacccaattcctcaccttcaagtcacttacagtctagtggaaacaaaacccaacacaatttcagttactgtctggtgctttgaagggagtggaacaggtgaacttgaggggcaggaggaagcagtttggtgagaaggtctcctggaggaggcgacggacaagaagggctcaatgggcttcattaggagctggcagaggacgttcctgggaacaggaacagagcatgcaaagggtccgaggcagagccccacttgaagggggatcggctgcagtgacagcttctaatacccacgacccactccttcaaccctcataaccgtgccttaaggggattgtgactggctccatttctttctctttttaattttttttttagatactgggtcacactctgtcagccaggttggagtgtagtggcacgatcatggcttactgcagcctcgaactctgggctccgcctatcctcctgcttcagccccctcaagtagctgggactacaggcatgcaccactatacccagctcatttttttttaacatttttgtagagatggggatctcactatgtggcccaggctagtctcaaactcctgacctcacactatccttctatcggcctcccaaagtgctgggattacaggtgtgagccaccacacctggcccagccccatttcccagatgaggaaagtgtggcacagagaggttagacaagttgccccaaggtgacacggctggcagaggagccagggagtcccaccccagagccctggattttgaccactctgctgatgggagggaggcatgagccggtgcacagtttatgaagtcgtgtaaactgagagcaggagttagaagtcagtcaaccatgtaatgggagtcctcaagggacagctaggcgtttctacagccaagcgcatatttggccccaagcacaagaaggcgcgcaacagataaaccagtgatcacttgattttgatttgcaagcaggcagtaggaaataaattgcaaaggtggaggccggatgcagtgtctcatgcctataatcccagcactttgggaggccaaggtgggcagatcacttgaggtcgggagtttgagaccagcctgaccaacacggagaaaccccgtctctactaaaaatacaaaattagctgggtgtggtggcgggcacctgtaatcccagctacttgggaggctaaggcatgagaatcatttgaacccaggaagcagaggttgcggtgagctgagatcgcgccattgcactccagcctgaacaacaagaatgaaactccgtctcaaaaaagaaaaatttgcaaaggtgagggtccatcctcattgctagggtgccctttgccctctgccctttgcccttcccctgccccaactcttctgtttttcagcaggaagagggtgggctgggctcaagcaggtggtggcaagtggctgacctgcaggtgggctctgtgtttgcaccagctgggctgttaggagaggcaggcgtgagacgaccccagctggggggtgttgaacttggcactatggggtgaggattaaccacagcagcccaggctacttctcagttcccttatcacctcctgaaccccacccccccagcaatgaatgttaataaaccccacccttcttccctccccctttccccgagctcactccagtcaagggagagagtctgacagtttaggtcaactgaggctaagccacaaaaagggcccctgcccccattcttgtggcacttgatgcgtttctgtgagtcctttatctcagctgacgtggatggcggtggttttgacagtatccctgctggtagccatttcctttttataaactgggaccctgaaaccagagaagtgaagggacttgccacaggtcacacagcgtataaggaccagggcaaaggggcggggataaaatcaagggctccatgctgcctccccacttggggcccaccactggcttccccatgggcgtaaaggagcaaaccaagttagaaggccaggcctgcaggtgcccaacaggaagggacaaggagccacagctgtcttgcctgtgacacaggccatccagccatgcccagagctaaccccctggctaagccccgaggcccagcttgactgctggcatctgttaccatggagacccaggctggcctgagggctgggccagtgatggcaggccctgtccccatggatagaaacaggtgcttgggctcagaggccttgagtggctcccactgtccccatggccagtgagtcccgacagcataaattggaaccgttacccacctttcgccccccagctgacacctccaccaacccaaggcctgagctgtcccctccacgtgtctgtgctctctttaatgccctgcctgggggctgggagtggtgaggatgtggatgtgaggttgaaggtttctcagggaatgagccagagctgccagaagaggcagagtgtaacccagactgcagatgatgggaagaacgcggaacagaagtgacctgaaggatccgcagggggaaagcagagaggtgggcacgcgggcacctggtaccttgtcccagccatgccaccagctagctgtgaggctttgggcgagtcccttgccctctctggctctcatccaaggaatgaggaagttggaacaaatgatgaatccctaagaccccttctaggtttgacattctttgagttgcattccaaaaccctggactcccccaggtaagcaaggccagggcttgccccatcctccccacacaagctcaggcagcacccactcctgggctgggttcccgaggaagagcctgcggagaggagaccccggagctgcctgcactggtcagtgcatgggggcaggggtggcagaccactttgtggattgatggagctcaggaaggtgagaagggacccacaggtgagagtttcgctcccctggtcatctctttaggtaaataaatccacatccgccacttccccttcccttcccaccctgggggcgctgagaactccagggagcccagagctgaggcctgagctctgcttgctcacactgggtcttccctcagagacccccaagccctcctatcttctgcagtcaccgtcatccacttttctgtagggagggaacagcatggagctctctgttcaccggtctccaggacctcggattccacctttaatcctgaaaacccaggaaggcttctgtatccctacaatgaagcaggtttggggctggatctgcagggtggcaactcaatccatgcagaacagaagaagcatggacttttccattctggctattccattcactagctgggccattctgagcaaactacctcccaatgtgcctcagtttcctcatctgcaaaatgggctagtcgctggcattgtaccgagcagtatgagaccggaggtcatgggaagaggctggtaggcacttcatcccaggcagctgctcagggacatgggacacagggaggggactccgagctgctcctagctcagagaggctctagggacaggcactggaaggaaggggatgcagaatggtgagtggagctgggtctcagaacacagacatcttgaagtctgctatgtgctgatttcacacttgaccccccaacaccctgaggcattactgtcctcatttctcagatggaactacagaggcccaaggaaaaggggcttaggccaggacacccagctcttctccaagtgtctgtctcaggctagcttttgcaactctccattgggattcttcctagatctccatctgtacctgccaacccacctctgacccccaacctgttgcgcccagtatttgctgccgatcaggacagccttaacccctccttcccgggcaatcagctgctccaagccaagcccacccctgccccctggagggaggcggctccttttaaggctgcttctgggaatttccactccagagccagaaccagagaccccagccccacttgacacctgcggctcacccttggggaggtggggcgccagacctgagtgcaggagacgcagacctggaagggctccccctccctgacctgccacacatcgagtttgtctgcgtcgagtttggccagtctgtgagggtcaggaatagagcaggagacagcagggccacctccttcagaaggcccccaccgctccatccctgc

SEQ ID NO: 11 - DNA, 6873 bp, mus musculus
ggatccctgttgcagtcataccctatgggaaaagagcaacttacctatcttaaggagattgggggaatttagatatttgtgcatcctctttcacttgaatgagaacaatgtaccagatcgtcaacagtgcacatttgacccggccagtagcaacatacctaaaactacctcaccattgtaaaacaccctaagaatcacaagaaacttacagtttttcagagacaaatcaaggagcagagttacagaattaaaaacaattcatcataccttagaattttctctaatcgaatgagttgataattgtttccatactcttagccatctttggagctatactttgaaggttaaaaatatcctagactaagttagttttcattgtaagtgctagcagcctttgctttctggtgtgaaataagaactaatttcaagtagaaagcacagagttcaggagagatgaagactatgttgcccaggctagtcctcagcccataactgggttcttgggctaaattctagctgctccattggaaataagcaagacagtgaattaagcacacacgaagagtactaaacgttgcgtggccaaggccgagaggctgagggtctgcctagtaacggtaacctttccctgatctctggtttatcgttttgaagaccttttcagaaggagtagtctgtctgtatgtccttccatcccaagcaagtgaagagcccagaggagccactggatatcaagctaggtctccaagcagttatgtctaaggagcttcaggctttgtttgcgaggttagaatggatgtagcttgtccagatgcctcctcagtgatgttgttgttgataggttgcccatgtgtttcttttcttaccaggtttccctgtggtctctgcttgtgaccctggtggttcttttcataatgactggcaccatgctgggacctgaactgctggcaagcattcccacaactgtttacgtggtcgccatttttatgcctctggcggctacgcctcgggttatggcttagctaccctcttcacctccgcccaactgcaagaggactgtgtgtctggaaacaggaagtcagaacgtgcagcttctgcactgcgattcttaaacttcccgcctcgctttataggtagcatgtacatgttcccttctgctctacgccttcttccagtctgccgaggcaggggtcttcgtgttgatctacaaaatgtacggaagtgagatactgcacaagcgagaggccctagacgaagacgaagacaccgatatttcttataagaaactgaaagaagaggaaatggcagacacctcctatggcaccgtggggacgcatgacttagtgatgatggagaccacccagaccgccctctgactgaggagatacacgggagctgaaacatcacttcctatttgtgaccattggtagcgagtatggttcgcatccgggataaagatgggttgacatttcctgtaacagatttgctcttcccactgtaatgtagtatctcagtattacccatgtgtttttctaactcaacagagtgtcccaatattgcttacacctggatcagctaaagtgccgcgtcctctgcttaagtagtgtgctgtttgtttgtttgttttttgttttgttttgtttttttccatttccaccagcattgctacagataggaaatggggttggaaatgtttgtaaaacagaaccatgggtttgttcaacttacaaacaaccgattctgttcagggcgagcctgtattgagaaaagtccaaaacgggtcaaaaagggttgaaacgacaggatagcattgcatcgtcaagccagagaaaaccgtattaatgtgtgtgactacttgatctagtatctattgttaatggccatcaacattgtgcaggggtgaaaggcatttttccccatatgtttcctgtatgtgtataaacgcatctcagctccatttatcgtctgaaggaatgatttacttaggaaaatgcgtagacctcacctcagggagagaaaatgggccactttgttcatccgtgggaaagggctgtggctacaggctttccttccggaaaggcctgtggctggacactgtcccactgctctggtagactggagctgtgatctgagacaacctaagaggttcagagcagtctcctaaccttggtattttgctccctaatcagacacactggcctcccttgtcttcttcatgacagacatctggagctacagacatgggggcccacctggctcggctaatctcggtgatgattctggggttgaattctcatctcatctagttcccctacaaatccttgctgtggctagcaaggaaagctctttttctgcatccacgagggagtgggggtgggggtcgcctcttaaccagtgtggggaaggttttgctcctcatggcaacagcaggtggtagggctttttctaccagtgcgcggccgcctatttaacgcagcgtggagggcagctgggctgcgctgatggctgcctgggcgggcgaggcgcgggacgcacccatgttcccggcgagcacgttccacccctgcccgcatccttatccgcaggccaccaaagccggggatggctggaggttcggagccaggggctgccgacccgcgcccccctccttcctccccggctacagacagctcatggccgcggagtacgtcgacagccaccagcgggcacagctcatggccctgctgtcgcggatgggtccccggtcggtcagcagccgtgacgctgcggtgcaggtgaacccgcgccgcgacgcctcggtgcagtgttcactcgggcgccgcacgctgcagcctgcagggtgccgagccagccccgacgcccgatcgggttcctgtcaaccccgtggccacgccggcgccgggagatccccgcgatcctggcagaccgtagccccgttctcgtccgtgaccttctgtggcctctcctcctcactggaggttgcgggaggcaggcagacacccacgaagggagaggggagcccggcatcctcggggacccgggaaccggagccgagagaggtggccgcgaggaaagcggtcccccagccgcgaagcgaggagggcgatgttcaggctgcagggcaggccgggtgggagcagcagccaccaccggaggaccggaacagtgtggcggcgatgcagtctgagcctgggagcgaggagccatgtcctgccgcagagatggctcaggaccccggtgattcggatgcccctcgagaccaggcctccccgcaaagcacggagcaggacaaggagcgcctgcgtttccaggtgaggccagcctgatggcctggacgcctccagaattgtagggctccttcagggctaagctggtggctctgggtgatgcagaacatagaattcttccatgccatccgtctggttttgtttgtttgtttgtttgtaacatgtttggtgttttgattgcatgttgtatctgtacacttcgttgtagtggagagatgggagcagaagagggtgtcggatccggatcccctgggactggcgttttacagatggttgtgagtcaccatgtgagttttaggatcggaattacggtcctctagaagaacagggtgttgtttcacagctgagccatctctccagctctttggcatataggattttgcagccgctgcctgttaatacaatgggaggcgtttacacaataaaaaccaacccatatgtgtcctgacccactggcagcctctgctcctggggaatgccagttgtaattattctgatcacataaacgctacacatgaggtctccgcggagaatgcgcacagtctgggtttggaccaaacttcagatggctgaaggaagataagtgcacacatggcagaaacataatcttttgaacttcgttgcggggagagtcggtttcccaaggctcctttttttatttcccctctagatgatctgtcttggttaacttgccggcttgttctataccagccccttcccttcgtttctgaagctgtcaactgaagcttctctctcccaaacttgcctggcttaaaaaacaaacaaacaaaaacaaaacaccccccccaaaaaaaaaaacaacaaaaaaaaaaaagaaaagaaaaagaaaaagaaataaaagaaaaaaaaaaccactctccccattcatcgaggccagccactgctaagctgttggatggtcttgagttgctgcctgtgctagcaaacaaggaggcacaaagagtgctgtaggtcgtatacccccaccaaagaaatggagagccctgagctccaggagaggactctgagacattccttgtttttcagtcatttcaaggctggtgtgtttgaggttggggtggcagtggaatggggtgtcagaaaaaatagaaaagtgcttggcggttgctgttcacagctgggtgtgatctcttaggcagaaatcccaagttttcgggcctctgtggtggtcgttcacctataaaaaattgcattaagagttcttccaagccctgccactcctaaagacttagttataaaaacttgtttccaacttgtttgtcactaagtgggaagcttgggaagtttaagaaccaggtgctaacactatgtagttcataccaaatgagctagacttgggtaggtagcgggactcttttggaaacttacctagcatcaaggaaaatttagtattggttgaagactttcaaaggttttagaagagcctttctctttcggcataacaactttcccatgtgtgagtgtcctaatgcatcgcccacataaaatgccacgggaagaatcccaaactctaaaccgcacgatttggcttctcccttgtctgaggggggaaaaaccacttatcggtctgctgctatatgaactatcttgtttggcctccgtttacatatttgtttgattgagctattagttcacctggttaacttagaggttgacccaagtctaaccttactaccacggtaatcttaaagtatcaagtggaatgtggtcccaggttctgaaaattagggtcactcgggcatcacttgcttaaagtctggtaccctgctgttcagttcttagagcagaagtacggctactatcactgcaaggactgcaaaatccggtgggagagcgcctatgtgtggtgtgtgcagggcaccagtaaggtaagagacaccgtgcagccctcctgctctgctgtgttgccgagtgtctgctccatgccgatgtctttctcctcgcaggtgtacttcaaacagttctgccgagtgtgtgagaaatcctacaacccttacagagtggaggacatcacctgtcaagtaaaccaaacgtttgcattttggaagaggggtttggtgcacgactttgagtatatttcctgaaggaggtggtttccagtagctttaggctctaccttttccctcctccttccttttcatttttgactaggttggtggtagaaagtcccctccactgtaaatggggtgtttactcccttctgctgttgtaaaacttgattgcatgccctctcttgcatctggttaccttgttagcagtagaaagggcttgcttacctggcttcttcccactcggacctaagggaaaacatattgcaaaacagagtgcctttctgctagcttgagatggtacacattaccccaatgctacataggaaacacattcccaagttagcatatgaaacacaagaaattgagctctggcttttcttgagagtttacaaagggagtttcctgtaagaccatcctacactgtctagctctatgcagtttacccataactgtggctaagagtttgcttgcttagtattaatttagcactgtgccaagggacttagataaccttgaaaacatttacctgttaaaattaatgacagagataaaggaattcgaattccacatctgagagcccagtgcacttaaagttggtaattggagaattaattaccttagggtgggccctgtgaaaccgagaatggaaagccactaaagactccatctagaaaaggggactgtagtcacttttctacaataaggggccttaaacttccctaagcttccctgcacttggttctcagtgcccagcacacaggccacttgttctgtaatctgttttgaagctccaagaatcgagtggagacagggctcaccctttgtacttttcactccgatttttcagaagttgtaaaagaactaagatgtgcctgcccagtcagacttcgccacgtggaccctaaacgcccccatcggcaagacttgtgtgggagatgcaaggacaaacgcctgtcctgcgacagcaccttcagcttcaaatacatcatttagtgagagtcgaaaacgtttctgctagatggggctaatggaatggacaagtgagctttctcccctcttcacctcttccctttccaaattcttcatgacagacagtgtacttggatataaagcctgtgaataaaaggtattgcaaacaagtttgaggctttatccaattcatgtgtcagtttgaggggtgcatgtgcggagagtcaataactttcttaacatttgttgatgagagtgagtcaggctgacttaaggaagttaaaggcacctcattcaacaattaagatttttctttctttttgtttagttttattttatttataaatatatgagtacactgtagctgtcttcagacacaccaaaagaaggcatcagatcccattacagatagttgtgagccaccatgtggttgctgggacttgaactccggacctctggaagagcagttggtaaacccctttcttaactgctgaaccatctctccagcccaaatcttaaggttttacagacaagaatattacagg

SEQ ID NO: 12 - DNA, 4090 bp, Mus musculus
  misc_feature (1)..(4090): N equals unknown
ggcgggcgaggcgcgggacgcacccatgttcccggcgagcacgttccacccctgcccgcatccttatccgcaggccaccaaagccggggatggctggaggttcggagccaggggctgccgacccgcgcccccctccttcctccccggctacagacagctcatggccgcggagtacgtcgacagccaccagcgggcacagctcatggccctgctgtcgcggatgggtccccggtcggtcagcagccgtgacgctgcggtgcaggtgaacccgcgccgcgacgcctcggtgcagtgttcactcgggcgccgcacgctgcagcctgcagggtgccgagccagccccgacgcccggtcgggttcctgtcaaccccgtggccacgccggcgccgggagatccccgcgatcctggcagaccgtagccccgttctcgtccgtgaccttctgtggcctctcctcctcactggaggttgcgggaggcaggcagacacccacgaagggagaggggagcccggcatcctcggggacccgggaaccggagccgagagaggtggccgtgaggaaagcggtcccccagccgcgaagcgaggagggcgacgttcaggctgcagggcaggccgggtgggagcagcagccaccaccggaggaccggaacagtgtggcggcgatgcagtctgagcctgggagcgaggagccatgtcctgccgcagagatggctcaggaccccggtgattcggatgcccctccccgcaaagcaccaagcaggacaaggagctcctgcgtttccaggtgaggccagcctggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntaccctgctgttcagttcttagagcagaagtacggctactatcactgcaaggactgcaaaatccggtgggagagcgcctatgtgtggtgtgtgcagggcaccagtaaggtaagagacaccgtgnnnnnnnnnnnnnnnnnnnnnnnnnnnnnnnnnnnnnnnnnnnnnnnnnnnnnnnnnnnnnnnnnnnnnnnnnnnnnntctttctcctcgtaggtgtacttcaaacagttctgccgagtgtgtgagaaatcctacaacccttacagagtggaggacgtcacctgtcaagtaaaccaaacgtttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngctctgagttttcagagttgtaaaggaactagatgtgcctgcccagtcagacctcgccacgtgtaccttagacgcccccatcagcaagacttgtgtgagagatgcaaggacaaacgcctgtcctgcgacagcaccgtcagcttcaaatacatgatttagtgagagtcgaaaacgtttctgctagatggggctaatggaatggacaagtgagctttctcccctcttcacctcttccctttccaaattcttcatgacagacagtgttacttggatataaagcctgtgaataaaaggtattgcaaaca

SEQ ID NO: 13 - DNA, 2075 bp, human
gttaaattgacatatcctgttgttcagttcttagagcagaaatatggctattaccactgcaaggactgcaacatccgctgggagagtgcttatgtgtggtgtgtacagggaactaacaaggtaagaaataccaggtaactggcatcttcttgctgaaagtgtcaaggcgattttaagtttatcctctttgtcatcacaggtttacttcaaacagttttgcagaacttgtcagaagtcttataacccttaccgagtggaggatatcacctgtcaagtaaatcagatgttttgcattttgtctgacctgggcagtcgtcgagggtttttagtatagtttgagtatacttccaaaaagaggccaggcccccagaccttaggtttcaactggcttttgttaggagtggtagaaacaatactcagctgggaaacggggccttggtgttagcttctttctggccttgcaaatcttgctgttgttaacctcttctaaaactgttaacctcacttgcaatatggaagaatacttgtcttacttgctacttagtctaatgtataagaaaatcaacaaaaacatgcttgtcagctaacatgaggtagtcaaggttgactgttttaccgaaacgcttcttatgaagcacaaccttaaagtacttaagcacagggggttagtttgtcttgcctgaaagctcacaaagggacagtttaagataaatctaagttgtctagctttatggggagttgactataatggtaagcaagcaatatgttaactaagcattgcttaagcgcttgcttgctattaactgtgctaaggggcttagctaatctttaagaggaaagaagtgactacattcgcctcctgtcacacagctaatggagtctgaattgccagttgagacagcctaatcaatacacttgacccacgttggatatttaaaagcattaacaccctggggtggtggagagaaactaagtatggaaagccacttagaatcacttagatcagagctgggcatgtttctaaaagaggatgccttaaccactctgctcttggtgttcattgtcaaattcatccctgacttgttctctaccctttctcttaaacagttgttgtaaaagaaatttcacaattcataattggatctgatgcaatatagcagcagtacagcatggttaaacacccactattcctagccctgtcattgctacgtaggtagggatgtagagggaaaacaagattactatgggaccttgcttagagcacattcattaagtacttgaatggactagaaaaatgttgaagtcctaggaaatcactaagggtttatcttctgcatgcccttctgtatttttttcccccagagttgtaaacaaacgagatgttcctgcccagtaaaacttcgccacgtggaccctaaacggccccaccgtcaagatttgtgcggtagatgcaaaggcaaacgcctgtcctgtgacagcactttcagcttcaaatacatcatttaggtgaaagtcagtgttgctgtgcatgcgctgatggagtagacgagtgagcttttccgtgcctctcctccacctctcccttctcaaaatacttcatgaaaggcagtgtattctgaaaaagccttcaaataaaggtattgcaacacgatttatacattgcataaaatctgtctttgaaaataaagtttcaagagcgcttgtcttgtgctaacagtctgggcctgtcacttcacctttatgaatgcttgctgatggcatagagtgggccaggctctgagttaggctgcagccacttggaaaacaatttaggggggtgcttgtagacgaggtctacttatttaggcaggtctggaggactgaagcttagaaggaagttaactgaataaaaagccgcctagcgatcgcgccactgcactccagcctgggtgacagagtgagactccatctcaaaaaaaaaagctgcctagctgtaacattaaggcattcttttgggagaggtggaggcagagccatttattggttgcatgagaccgttggaggttaacgttgagtaagaatgctgagtggcggtg

SEQ ID NO: 14 - DNA, 1118 bp, human
ggggggagctgggacctaagccgcgcgcacacccctttctctgcgtctggtggaggtgcacagaggcttttgagtcaggcccaagcgcagccaggtgacctccccgcggcctttcaagcctgagctcggtggacagctccctctcccgtgagtcccgctgtcctgtacgcgcccggtcgagccccgggctgcgcaccccgctaggagctgcccggccagcccgcttctctgcccggagccatgaatctcagtagcgccagtagcacggaggaaaaggcagtgacgaccgtgctctggggctgcgagctcagtcaggagaggcggacttggaccttcagaccccagctggaggggaagcagagctgcaggctgttgcttcatacgatttgcttgggggagaaagccaaagaggagatgcatcgcgtggagatcctgcccccagcaaaccaggaggacaagaagatgcagccggtcaccattgcctcactccaggcctcagtcctccccatggtctccatggtaggagtgcagctttctcccccagttactttccagctccgggctggctcaggacccgtgttcctcagtggccaggaacgttatgaagcatcagacctaacctgggaggaggaggaggaagaagaaggggaggaggaggaagaggaagaggaagatgatgaggatgaggatgcagatatatctctggaggagcaaagccctgtcaaacaagtcaaaaggctggtgccccagaagcaggcgagcgtggctaagaaaaaaaagctggaaaaagaagaagaggaaataagagccagcgttagagacaagagccctgtgaaaaaggccaaagccacagccagagccaagaagccaggattcaagaaatgaggagccacgccttggggggcacggtgcaaagtgggccttccctgggctgtgctgcaggcacagggtgcccctgtccagcccctccacctgtgtctgaatgcaacaggggtgttgcgggggcaacatgagagcccctcacccccaactctccactttcaggaggcccccagtgaagagccccacctcggggtcacaataaagttgcctggtcaggaaaaaaaaaaaaaaaaaaa

SEQ ID NO: 15 - protein, 200 aa, xeniopus laevis
MASTVSNTSKLEKPVSLIWGCELNEQDKTFEFKVEDDEEKCEHQLALRTVCLGDKAKDEFNIVEIVTQEEGAEKSVPIATLKPSILPMATMVGIELTPPVTFRLKAGSGPLYISGQHVAMEEDYSWAEEEDEGEAEGEEEEEEEEDQESPPKAVKRPAATKKAGQAKKKKLDKEDESSEEDSPTKKGKGAGRGRKPAAKK

SEQ ID NO: 16 - protein, 103 aa, human
FLEQKYGYYHCKDCNIRWESAYVWCVQGTNKVYFKQFCRTCQKSYNPYRVEDITCQSCKQTRCSCPVKLRHVDPKRPHRQDLCGRCKGKRLSCDSTFSFKYII

SEQ ID NO: 17 - DNA, 27 bp, Mus musculus
gcaaagaagccagtgaccaagaaatga

SEQ ID NO: 18 - DNA, 27 bp, Mus musculus
cctgatcatgcaaattttattgtggcc

SEQ ID NO: 19 - protein, 18 aa, Mus musculus
KRPHRQDLCGRCKDKRLS

SEQ ID NO: 20 - DNA, 24 bp, Mus musculus
ctagaaaaggggactgtagtcact

SEQ ID NO: 21 - DNA, 24 bp, Mus musculus
tgcatctcccacacaagtcttgcc

SEQ ID NO: 22 - DNA, 24 bp, Mus musculus
ctagaaaaggggactataggcacc

SEQ ID NO: 23 - DNA, 24 bp, Mus musculus
tgcatctctcacacaagtgttgct